(19) United States
(12) Patent Application Publication
(10) Pub. No.: US 2003/0064954 A1
(43) Pub. Date: Apr. 3, 2003

(54) Cationic amphiphilic 1,4-dihydropyridine derivatives useful for delivery of nucleotide containing compounds

(76) Inventors: Arto Urtti, Kuopio (FI); Zanna Hyvonen, Kuopio (FI); Aiva Plotniece, Latvia (LV); Natalia Makarova, Latvia (LV); Inese Reine, Latvia (LV); Gunars Tirzitis, Latvia (LV); Brigita Vigante, Latvia (LV); Brigita Cekavicus, Latvia (LV); Andulis Shmidlers, Latvia (LV); Aivars Krauze, Latvia (LV); Raivis Zhalubovskis, Latvia (LV); Gunars Duburs, Latvia (LV); Mikko Turunen, Kuopio (FI); Seppo Yla-Herttuala, Vuorela (FI); Ilpo Jaaskelainen, Kuopio (FI); Marjo-Riitta Toppinen, Kyviarvi (FI)

Correspondence Address: KUBOVCIK & KUBOVCIK, SUITE 710, 900 17TH STREET NW, WASHINGTON, DC, 20006

(21) Appl. No.: 10/204,640

(22) PCT Filed: Feb. 22, 2001
(86) PCT No.: PCT/FI01/00185

(30) Foreign Application Priority Data
Feb. 23, 2000 (FI) 20000421

(51) Int. Cl. A61K 48/00; C12N 15/85

(52) U.S. Cl. 514/44; 514/332; 514/255.05; 435/455

(57) ABSTRACT

The present invention discloses amphiphilic 1,4-dihydropyridine derivatives useful for the preparation of a composition for delivering nucleotide containing compounds into a target cell and/or its nucleus. Said composition comprises 1,4-dihydropyridine derivatives having a good DNA condensing capacity and capability of self-association. Also disclosed are composition comprising said derivatives complexed with nucleotide containing compounds as well methods for the producing of said complexes. The invention is also related to the use of said 1,4-dihydropyridine derivatives for manufacturing systems for delivering nucleotide containing compounds useful in gene therapy and DNA vaccination.

### THE TECHNICAL FIELD OF THE INVENTION

The invention is related to amphiphilic 1,4-dihydropyridine derivatives useful for delivering genes, i.e. transporting nucleotide containing compounds into a target cell and its nucleus. Also disclosed are compositions comprising said derivatives as well as methods for producing complexed compositions made of said derivatives with nucleotide containing compounds. The invention is also related to the use of said 1,4-dihydropyridine derivatives for manufacturing delivery systems for nucleotide containing compounds as well as methods for in vitro and in vivo transportation of nucleotide containing compounds into a target cell and its nucleus.

### THE BACKGROUND OF THE INVENTION

Successful introduction of exogeneous nucleotide containing compounds into target cells is a prerequisite in gene therapy; as well as in other gene technology applications. For example, in gene therapy and/or DNA vaccination, nucleotide containing compounds, including DNA, RNA or their modified forms must be able to penetrate first into the cytoplasm and thereafter into the nucleus of the cell in an unchanged or intact form. The problem is that nucleotide containing compounds in addition to their large molecular weight are hydrophilic. This property prevents their effective entry into the cell and its nucleus. Furthermore, nucleotide containing compounds are prone to enzymatic degradation and inactivation -in the cells of a living organism. For that reason, novel effective delivery systems are essential for successful gene therapy, DNA vaccination and/or administration of gene-based drugs.

Viral vectors are currently the most effective vehicles in gene delivery. However, they have som disadvantages, such as risk of oncogenecity, immune responses and difficulties in industrial validation and upscaling. Problems related to viral vectors have prompted the search of efficient and safe non-viral delivery systems.

The medical applicability of drugs based on compounds containing nucleotides is difficult due to the poor transfer of these agents across the cell membranes. Cationic liposomes, including dioleylphosphatidylethanolamine (DOPE), N-(1-(2,3-dioleoyloxy)propyl)-N,N,N-trimethylammoniummethyl-sulfate (DOTAP), dioctadecylglycerospermine (DOGS) and (DOTMA), a cationic liposome present e.g. in the commercially available Lipofectin^{R}, have been used for gene transfer. Said compounds bind negatively charged nucleotide containing compounds with their positive charges and the complexes formed, bind to the cells and thereafter, deliver nucleotide containing compounds into the cells via endocytosis. The efficacy of the gene transfer is, however, not optimal because DOTAP and Lipofectin^{R} are non-selective vehicles. One reason for this is their binding to proteins, glycosaminoglycans and their non-selective interaction with cellular lipid bilayers. Furthermore, these non-selective vehicles do not provide optimal intracellular distribution of transgene due to entrapment of the complexes in the endosomal compartment. Escape of DNA from endosomes can be facilitated with pH-selective fusion properties of the vehicle or by the buffering capacity of the vehicles.

For the above discussed reasons, alternative effective delivery systems would be essential for successful gene therapy, DNA vaccination and/or administration of gene-based drugs. Clearly, a need exists for developing alternative nucleic acid delivery systems for use in different applications of the new applications in gene therapies and DNA-vaccination, etc. The objectives of the present invention is to provide improved more safe and efficient delivery systems with better buffering capacity.

1,4-Dihydropyridine derivatives are known mostly as calcium channel blockers in therapy of cardiovascular diseases. Other physiological features include antioxidant, radioprotective, antibacterial and membranotropic effects. Conventional 1,4-dihydropyridine derivatives have been used for delivering drugs. However, said drug delivering 1,4-dihydropyridine derivatives are not amphiphilic and they are not capable of self-association. Accordingly, they are not capable of complex formation with nucleotide containing compounds, which is a prerequisite in the present invention. The conventional dihydropyridine lack the relatively long alkyl chains, the preferred number of carbon atoms being ten or more, most preferably at least twelve, which is typical of the 1,4-dihydropyridines of the present invention. The net surface charge (25-49 mV) and the long alkyl chains, which characterize the 1,4-dihydropyridine derivatives of the present invention gives them their unique self-associating properties and make them useful for delivery of nucleotide containing compounds.

### THE SUMMARY OF THE INVENTION

In the present invention, novel amphiphilic cationic 1,4-dihydropyridine derivatives effective as DNA and/or RNA vehicles and transfection agents are disclosed. Based on surprising preliminary observations that some of the 1,4-dihydropyridine derivatives formed vesicular structures in water, the present inventors synthesized a multitude of cationic, amphiphilic compounds based on the 1,4-dihydropyridine derivative structures of the present invention, tested the properties of the compounds in order to find new efficient and safe compositions for gene delivery and studied the biophysical characteristics of the cationic amphiphile/DNA complexes and their transfection efficacies. During the studies it was demonstrated that a whole group of new compounds could be prepared, which compounds were demonstrated to be able to complex DNA and show very high transfection efficacy in vitro. Furthermore, some structural features, which are important for obtaining the desired transfection activity were revealed.

The characteristic features of the structures of the derivatives of the present invention their properties and the preparation of said cationic, amphiphilic compounds of the present invention are as defined in the claims.

### THE BRIEF DESCRIPTION OF THE FIGURES

FIG. 1 is a graphic depiction showing the influence of charge ratio on the size of complexes.

FIG. 2 shows the ability of 1,4-dihydropyridine derivatives to complex DNA.

FIG. 2A specifically shows gel electrophoresis of derivative V/plasmid DNA complexes at +/−16-0,125 charge ratios;

FIG. 2B specifically shows gel electrophoresis of derivative V:DOPE(1:1)/plasmid DNA complexes at +/−16-0,125;

FIG. 2C specifically shows gel electrophoresis of derivative XXIII/plasmid DNA complexes at +/−16-0,125 charge ratios.

FIG. 3 depicts the DNA condensation ability of cationic amphiphiles.

FIG. 3A shows condensation of derivatives XXII, XXIII, XXIV and XXV, respectively, DOTAP Lipofectin^{R}.

FIG. 3B shows DNA condensation ability of derivatives I, V, VI and VII, respectively.

FIG. 3C shows DNA condensation ability of derivatives I, V, VI and VII, respectively in combination with (DOPE) (1:1) (molar ratios).

FIG. 4 shows the transfection efficiencies of derivative XXIII, and DOTAP at carrier/plasmid DNA charge ratios 8-2, when transferred into CV1-P and D407 cell lines. Transfection efficiencies are given as percentage in comparison to transfection efficiency of Lipofectin^{R}. Each bar represents the average transfection efficiency from at least three experiments.

FIG. 4A shows efficiencies of transfection of CVP-P cells using derivative XXIII and DOTAP in comparison to Lipofectin^{R}.

FIG. 4B shows efficiencies of transfection of D407 cells using derivative XXIII and DOTAP in comparison to Lipofectin^{R}.

FIG. 5A shows the effect of serum, DOPE and pegylated lipid on transfection into subconfluent CV1-P cells. The complexes were prepared in Mes-Hepes buffer.

FIG. 5B shows the effect of serum, DOPE and pegylated lipid on transfection into subconfluent CV1-P cells. The complexes were prepared in 5% glucose.

### THE DETAILED DESCRIPTION OF THE INVENTION

The present inventors have found that some cationic, amphiphilic 1,4-dihydropyridine derivatives, especially double-charged derivatives, possess a DNA condensing capacity, which is valuable for successful delivery of nucleotide containing compounds. These derivatives show surprisingly efficient gene transfer capacity. In addition many of these derivatives demonstrate a buffering capacity of endosomal pH. This prevention of acidification of endosomes during transfection may protect the gene(s) from degrading enzymes.

The cationic, amphiphilic 1,4-dihydropyridine derivatives of the present invention are characterized by self-association and a capacity of condensation, i.e. complex-formation with nucleotide containing compounds, including DNA, RNA and/or their modified forms in plasmids, vectors, chimeric DNA/RNA constructs, etc., either as such or in different combinations. Therefore, the 1,4-dihydropyridine derivatives of the present invention are useful as vehicles, when transporting nucleotide containing compounds into target cells. The derivatives are useful in a wide variety of medical applications producing different routes of administration alone or in combination with other cationic liposomes lacking a 1,4-dihydropyridine structure. Such compounds are, for example, DOPE, DOTAP, DOT-MA, DOGS, etc. or cationic polymers such polyethylene imine (PEI), etc. The complexes made of the derivatives can for example be combined with surfactants, polymers and compounds which prolong the half-life in blood circulation, such as polyethylene imine (PEG) or fragments thereof.

As said above some dihydropyridine derivatives have been used as calcium channel antagonists in the treatment of hypertension. Furthermore, some dihydropyrine esters have been developed for improved delivery of the calcium channel blockers. These small molecules permeate through the cell membranes by simple diffusion. They do not have the self-associating properties that are crucial for complexation of DNA and other gene based drugs. The self-associating structures of the 1,4-dihydropyridine derivative of the present invention stabilize the complexes by creating the adequate electrostatic field and the weak molecular interactions keep the complexes intact. The complexes are taken up by the cells via endocytosis, not by simple diffusion.

The applications include, but are not limited to, DNA vaccination, gene therapy (ex vivo and in vivo) or delivery of other gene based drugs or gene based treatment modalities, including the use of sense, antisense nucleotide sequences, antigens, antibodies, ribozymes, as well as chimeric oligonucleotides constructs for gene correction. These may include DNA or RNA fragments, which code functionally active or inactive or conditionally inactivatable proteins. The cationic, amphiphilic derivatives of the present inventions are useful as reagents or for preparing reagents for transfection of the cells in laboratory settings.

Amphiphilic 1,4-dihydropyridine Derivatives

The amphiphilic 1,4-dihydropyridine derivatives of the present invention, which are disclosed below, are characterized by self-association and a good DNA condensing capacity which is provided by their positive surface charge (25-419 mV) and long alkyl chains comprising preferably 10-14 carbon atoms. Said 1,4-dihydropyridine derivatives effectively introduce nucleotide containing compounds, especially different forms of DNA and/or RNA into cells both in vitro and in vivo. The 1,4-dihydropyridine derivatives of the present invention have the capacity to complex and condense plasmid DNA. Furthermore, they show high gene transfer efficacy at low levels of toxicity. Thus, the present invention is related to compositions comprising nucleotide containing compounds complexed with the compounds defined below with or without surfactants and other compatible additives, such as other cationic liposomes, fusogenic peptides, targeting agents, antibodies etc.

The 1,4-dihydropyridine derivatives of the present invention are further characterized by having the general formula I,

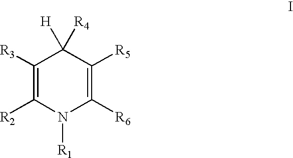

wherein

R₁ is hydrogen, (C₁-C₁₆), preferably (C₈C₁₄), most preferably (C₁₀-C₁₂)alkyl, aralkyl or aryl, selected from a group consisting of phenyl, substituted phenyl, naphthyl, acylCO(C₁-C₁₆), preferably (C₈-C₁₄), most preferably (C₁₀-C₁₂)alkyl and COaryl;

R₂ is (C₁-C₁₀), preferably (C₁-C₃)alkyl or CH₂X;

wherein

X is pyridinio (C₅H₅N⁺), substituted pyridinio, diazinio (C₄H₄N₂⁺), substituted diazinio, trialkyl(C₁-C₁₀), preferably (C₁-C₃)ammonio, a (C₁-C₁₆), preferably (C₈-C₁₄), most preferably (C₁₀-C₁₂)alkylthio group or an alkylthio group with a carbonyl function, selected from a group consisting of S(CH₂)ₙCONH₂, S(CH₂)ₙCOAr and S(CH₂)ₙCOO(C₁-C₁₆), preferably (C₈-C₁₄), most preferably (C₁₀-C₁₂)alkyl;

wherein

n is an integer from 1 to 16, preferably 8-14, most preferably 10-12;

R₃ is a cyano or nitril group or C(═Y)—(Z)ₙR₇ with a carbonyl function;

wherein

Y is O or S;

Z is O, S or NH or NR₇;

n is an integer 0 or 1; and

R₇ is saturated or unsaturated (C₁-C₁₈), preferably (C₈-C₁₄), most preferably (C₁₀-C₁₂)alkyl, a derivative of cyclohexane, a terpene selected from a group consisting of bornyl, i-bornyl, menthyl, steryl, cholesteryl, adamantyl, aralkyl(C₁-C₃)alkylAr;

wherein

Ar means aryl, alkoxyalkyl(C₁-C₆), preferably (C₁-C₃)alkyl-O—(C₁-C₃)alkyl, alkanoyloxyalkyl(C₁-C₃)alkyl[O—CO(C₅-C₁₆), preferably (C₈-C₁₄), most preferably (C₁₀-C₁₂)alkyl]ₙ;

wherein

n is an integer 1 or 2; or

a derivative of ammonioalkyl(C₁-C₃)alkylPy;

wherein

Py means a pyridinium or a (C₁-C₃) alkylN⁺tri (C₁-C₁₀), preferably (C₃-C₆)alkyl;

R₄ is H, (C₁-C₁₆), preferably (C₈-C₁₄), most preferably (C₁₀-C₁₂)alkyl or an alkyl group with a carbonyl function, selected from a group consisting of COO(C₁-C₁₀), preferably (C₃-C₆)alkyl, COOsteryl, aryl and C₆H₄R₈;

wherein

R₈ is H, Cl, Dr, I, CH₃, OCH₃, N(CH₃)₂, NO₂, OCHF₂; or a heteryl group preferably a pyridinium C₅H₄N⁺R₉;

wherein

R₉ is (C₁-C₁₆), preferably (C₈-C₁₄), most preferably (C₁₀-C₁₂) alkyl, aryl, aralkyl, alkoxycarbonylalkyl, cycloalkylcarbonylalkyl, (C₁-C₁₂), preferably (C₃-C₉), most preferably (C₅-C₇)alkylCOR₁₀ with a carbonylalkyl function;

wherein

R¹⁰ is NH₂, O(C₁-C₁₆), preferably (C₈-C₁₄), most preferably (C₁₀-C₁₂)alkyl, aryl, O-steryl, OH, O—, or COR₁₁; wherein R¹¹ is OH, O—, O(C₁-C₁₆), preferably (C₈-C₁₄), most preferably (C₁₀-C₁₂)alkyl, O-aryl or N(R₁₂)₂;

wherein

R₁₂ is H, (C₁₀-C₁₆), preferably (C₈-C₁₄), most preferably (C₁₀-C₁₂) alkyl, pyridiniumalkyl, ammoniumalkyl, carbalkoxyalkyl or carboxyalkyl;

R₅ is ammonio, a pyridinio selected from a group consisting of a C₅H₅N⁺—, a [(C₁-C₁₀), preferably (C₃-C₉), most preferably (C₅-C₇)alkyl]₃N⁺, a (C₁-C₁₆), preferably (C₈-C₁₄), most preferably (C₁₀-C₁₂)alkoxycarbonylmethylpyridyl or a C(═Y)—(Z)ₙR₇ with a carbonyl function;

wherein

Y is C or S;

Z is O, S or NH;

n is an integer 0 or 1;

R₇ is as defined above; and

R₆ is (C₁-C₁₀), preferably (C₃-C₆)alkyl, CH₂X;

wherein

X is pyridinio selected from a group consisting of pyridinio (C₅H₅N⁺), substituted pyridinio, trialkyl(C₁-C₁₀), preferably (C₃-C₆)ammonio and aryl;

with the provision that at least one, preferably two of the substituents R₁—R₆ comprise a carbon chain having at least 10 carbon atoms and/or at least one, preferably two positively charged pyridino groups.

R₂ is conveniently the same as R₆ and R₃ the same as R₅

Optionally R₅ and R₆ may taken together form a dioxosulfaindeno group SO₂C₆H₄; and/or R₁ and R₂ taken together form a carbonylmethylthio group.

In the 1,4-dihydropyridine derivatives of the present invention each ammonium and/or pyridinium group is provided with a counterion W—, wherein W means a halide, selected from a group consisting of I, Br and Cl; a perchlorate (ClO₄), a sulfate (1/2SO₄), a phosphate (1/3PO₄ or H₂PO₄).

Substituent combinations, which based on their structures may have the desired DNA condensing capacity properties, which can be measured by EtBr displacement assays (Ruponen, M. et al., Biochem. Biophys. Acta, 1415: 331-341, 1999) are listed in Table 1.

TABLE 1

| R¹ | R² | R³ | R⁴ | R⁵ | R⁶ |
|---|---|---|---|---|---|
| H | CH₂Py⁺Br⁻ | COOCH₃ | C₆H₄NO₂-2 | COOCH₃ | CH₂Py⁺Br⁻ |
| H | CH₂Py⁺Br⁻ | COOCH₃ | C₆H₄OCHF₂-2 | COOCH₃ | CH₂Py^{+Br}⁻ |
| H | CH₂N⁺(C₂H₅)₃Br⁻ | COOCH₃ | C₆H₄OCHF₂-2 | COOCH₃ | CH₂N⁺(C₂H₅)₃Br⁻ |
| H | CH₂N⁺(C₂H₅)₃Br⁻ | COOC₂H₅ | C₆H₅ | COOC₂H₅ | CH₂N⁺(C₂H₅)₃Br⁻ |
| CH₃ | CH₂Py⁺Br⁻ | COOCH₃ | C₆H₄OCHF₂-2 | COOCH₃ | CH₂Py⁺Br⁻ |
| CH₃ | CH₂Py⁺Br⁻ | COOC₁₂H₂₅ | C₆H₅ | COOC₁₂H₂₅ | CH₂Py⁺Br⁻ |
| C₆H₅ | CH₂Py⁺Br⁻ | COOC₁₂H₂₅ | C₆H₅ | COOC₁₂H₂₅ | CH₂Py⁺Br⁻ |
| H | CH₂Py⁺Br⁻ | COSC₁₂H₂₅ | Py⁺C₁₆H₃₃Br⁻ | COSC₁₂H₂₅ | CH₂Py⁺Br⁻ |
| H | CH₂Py⁺Br⁻ | CONHC₁₂H₂₅ | C₆H₅ | CONHC₁₂H₂₅ | CH₂Py⁺Br⁻ |
| H | CH₃ | COOAd-1 | Py⁺C₁₆H₃₃Br⁻ | SO2C6H4 | |
| H | SCH₂CONH₂ | CN | C₆H₄NO₂-3 | 4-Py⁺CH₂COOC₂H₅Br⁻ | CH₃ |

| | | | | |
|---|---|---|---|---|
| —COCH₂S— | CN | C₆H₄NO₂-3 | 4-Py⁺CH₂COOC₂H₅Br⁻ | CH₃ |

Even if the alkyl groups of the substituents of the 1,4-dihydropyridine of the present invention are indicated to include anything from one carbon atom it is preferable that one or more substituents comprise a straight or cyclic alkyl chain having at least 6 carbon atoms, preferably at least 8 carbon atoms, more preferably at least 10 carbon atoms.

Generally, less than 18 carbon atoms, more preferably less than 16 carbon atoms, most preferably less than 14 carbon atoms are required. Two long chains seems to suffer from a certain stiffness, which disturbs the DNa condensation properties.

Most preferably the carbon chain comprises so many carbon atoms that the desired characteristics of the 1,4-dihydropyridine derivatives of the present invention are achieved, said characteristics being self-association and DNA condensation capacity, i.e. complexation with nucleotide containing compounds, such as DNA, RNA and/or their modified forms as such, in plasmids, vectors, constructs etc.

More specially the most preferred 27 derivatives with the code numbers I-XXVII and having the general formula I and their substituents are shown in Table 2, which also refers to the examples in which their respective preparation and chemical properties are described.

TABLE 2

| Derivatives | | | |
|---|---|---|---|
| Exam./Code No. | R₂ = R6 | R₃ = R₅ | R₄ |
| 2l/I* | —CH₃ | —COO(CH₂)₂OCOC₁₅H₃₁ | Py⁺—CH₃I⁻ |
| 2k/II | —CH₃ | —COO(CH₂)₃OC₁₆H₃₃ | Py⁺—CH₃I⁻ |
| 2m/III | —CH₃ | —COO(CH₂)₃OCOC₁₅H₃₁ | Py⁺—CH₃I⁻ |
| 2g/IV | —CH₃ | —COOC₉H₁₉ | Py⁺—CH₃I⁻ |
| 2h/V* | —CH₃ | —COOC₁₂H₂₅ | Py⁺—CH₃I⁻ |
| 2i/VI* | —CH₃ | —COOC₁₄H₂₉ | Py⁺—CH₃I⁻ |
| 2j/VII | —CH₃ | —COOC₁₆H₃₃ | Py⁺—CH₃I⁻ |
| 2e/VIII | —CH₃ | —COO(CH₂)₂OC₃H₇ | Py⁺—CH₃I⁻ |
| 2n/IX | —CH₃ | —COOCH₂CH(OCOC₁₅H₃₁)CH₂(OCOC₁₅H₃₁) | Py⁺—CH₃I⁻ |
| 2o/X | —CH₃ | —COO-menthyl | Py⁺—CH₃I⁻ |
| 2s/XI | —CH₃ | —COO-bornyl | Py⁺—CH₃I⁻ |
| 2r/XII | —CH₃ | —COO-cholesteryl | Py⁺—CH₃I⁻ |
| 6a/XIII | —CH₃ | —COOC₂H₅ | Py⁺—C₉H₁₉Br⁻ |
| 6b/XIV | —CH₃ | —COOC₁₄H₂₉ | Py⁺—C₉H₁₉Br⁻ |
| 8a/XV | —CH₃ | —COOC₂H₅ | Py⁺—C₁₆H₃₃Br⁻ |
| 8d/XVI | —CH₃ | —COO(CH₂)₂OC₃H₇ | Py⁺—C₁₆H₃₃Br⁻ |
| 8c/XVII | —CH₃ | —COOC₁₄H₂₉ | Py⁺—C₁₆H₃₃Br⁻ |
| 8g/XVIII | —CH₃ | —COO-menthyl | Py⁺—C₁₆H₃₃Br⁻ |
| 3a/XIX | —CH₃ | —COO(CH₂)₂OC₃H₇ | Py⁺—C₃H₇I |
| 11c/XX* | —CH₃ | —COOC₁₆H₃₃ | Py⁺—CH₂CONH₂I⁻ |
| 4a/XXI | —CH₃ | —COOC₁₄H₂₉ | Py⁺—C₄H₉Br⁻ |
| 22a/XXII | —CH₂Py⁺Br⁻ | —COOC₁₀H₂₁ | Ph |
| 22b/XXIII* | —CH₂Py⁺Br⁻ | —COOC₁₂H₂₅ | Ph |
| 22c/XXIV* | —CH₂Py⁺Br⁻ | —COOC₁₄H₂₉ | Ph |
| 22d/XXV | —CH₂Py⁺Br⁻ | —COOC₁₆H₃₃ | Ph |
| 22e/XXVI | —CH₂Py⁺Br⁻ | —COOC₁₈H₃₇ | Ph |
| 23a/XXVII* | —CH₂N⁺(CH₃)₂C₈H₁₇Br⁻ | —COOC₁₀H₂₁ | Ph |

*means especially good DNA condensing capacity (Ruponen, et al., Biochem. Biophys. Acta 1415:331-341, 1999). Py⁺ means pyridinium; Ph means phenyl

The derivatives of the present invention include derivatives selected from a group of 1,4-dihydropyridine derivatives having the general formula I, wherein the substituents may be the following:

R₂ is either the same as or different from R₆ and is methyl, pyridiniomethylbromide, trialkylammoniomethylbromide, carbamoylmethylthio or alkylcarbamoylmethylthio;

R₃ is either the same as or different from R₅ and is octyloxypropyloxycarbonyl, nonyloxypropyloxycarbonyl, decyloxypropyloxycarbonyl, undecyloxypropyloxycarbonyl, dodecyloxypropyloxycarbonyl, tridecyloxypropyloxycarbonyl, tetradecyloxypropyloxycarbonyl, pentadecyloxypropyloxycarbonyl, hexadecyloxypropyloxycarbonyl, octyloxypropyloxycarbonyl, nonyloxypropyloxycarbonyl, decyloxypropyloxycarbonyl, undecyloxypropyloxycarbonyl, dodecyloxypropyloxycarbonyl, tridecyloxypropyloxycarbonyl, tetradecyloxypropyloxycarbonyl, pentadecyloxypropyloxycarbonyl, hexadecyloxypropyloxycarbonyl, pentadecyloxycarbonylpropyloxycarbonyl, octadecyloxycarbonyl, nonyloxycarbonyl, decyloxycarbonyl, undecyloxycarbonyl, dodecyloxycarbonyl, tridecyloxycarbonyl, tetradecyloxycarbonyl, pentadecyloxycarbonyl, hexadecyloxycarbonyl, propyloxyethyloxycarbonyl, (2,3-dipentadecyloxycarbonyl)propyloxycarbonyl, menthyloxycarbonyl, bornyloxycarbonyl, cholesteryloxycarbonyl, ethyloxycarbonyl, propyloxycarbonyl, cyclohexyl[2-isopropyl]carbonyl, decyloxycarbonyl, ethylthiocarbonyl, dodecylthiocarbonyl, carbamoyl, hexadecylamidocarbonyl, diethylamidocarbonyl, morpholidocarbonyl, pyridyl, pyridinium, pyridinio, triethylammonio, trioctylammonio, dimethyloctylammonio, triethylammonioethoxycarbonyl, dimethyloctylammonioethoxycarbonyl, pyridinioethoxycarbonyl, benzylamidocarbonyl;

R₄ is iodomethylpyridinium, bromononylpyridinium, bromohexadecylpyridinium, iodopropylpyridinium, iodocarbamoylmethylpyridinium, bromobutylpyridinium, phenyl, iodoacetonylpyridinium, bromonaphtacylpyridinium, bromoethoxycarbonylmethylpyridinium, bromophenacylpyridinium, ethoxycarbonylethylcarbamoyl, pyridinioethylamidocarbonyl or diethylcarbamoyl; and

R¹ is hydrogen, methyl, ethyl, butyl, dodecyl or benzyl.

The most preferred 1,4-dihydropyridine derivatives are the following:

Derivative I (Example 21)

1-Methyl-3-(2′,6′-dimethyl-3′,5′-dipentadecyloxycarbonyl-ethoxycarbonyl-1′,4′-dihydropyridyl-4′)-pyridinium iodide or 1′,4′-Dihydro-1,2′,6′-trimethyl-3′,5′-bis[(2-palmitoyloxyethoxy)carbonyl]-3,4′-bipyridinium iodide (IUPAC name);

Derivative II (Example 2k)

1-Methyl-3-(2′,6′-dimethyl-3′,5′-dihexadecyloxypropyloxycarbonyl-1′,4′-dihydropyridyl-4′)-pyridinium iodide or 3′,5′-Bis[(3-hexadecyloxypropoxy)carbonyl]-1′,4′-dihydro-1,2′,6′-trimethyl-3,4′-bipyridinium iodide (IUPAC name);

Derivative III (Example 2m)

1-Methyl-3-(2′,6′-dimethyl-3′,5′-dipentadecyloxycarbonylpropyloxycarbonyl-1′,4′-dihydropyridyl-4′)-pyridinium iodide or 1′,4′-Dihydro-1,2′,6-trimethyl-3,S5′-bis[(3-palmitoyloxypropoxy)carbonyl]-3,4′-bipyridinium iodide (IUPAC name);

Derivative IV (Example 2g)

1-Methyl-3-(2′,6′-dimethyl-3′,5′-dinonyloxy-carbonyl-1′,4′-dihydropyridyl-4′)-pyridinium iodide or 1′,4′-Dihydro-1,2′,6′-trimethyl-3′,5′-bis(nonyloxycarbonyl)-3,4′-bipyridinium iodide (IUPAC name);

Derivative V (Example 2h)

1-Methyl-3-(2′,6′-dimethyl-3′,5′-didodecyloxycarbonyl-1′,4′-dihydropyridyl-4′)-pyridinium iodide or 3′,5′-Bis(dodecyloxycarbonyl)-1′,4′-dihydro-1,2′,6′-trimethyl-3,4′-bipyridinium iodide (IUPAC name);

Derivative VI (Example 2i)

1-Methyl-3-(2′,6′-dimethyl-3′,5′-ditetradecyloxycarbonyl-1′,4′-dihydropyridyl-4′)-pyridinium iodide or 1′,4′-Dihydro-1,2′,6′-trimethyl-3′,5′-bis(tetradecyloxycarbonyl)-3,4′-bipyridinium iodide (IUPAC name);

Derivative VII (Example 2j)

1-Methyl-3-(2′,6′-dimethyl-3′,5′-dihexadecyloxycarbonyl-1′,4′-dihydropyridyl-4′)-pyridinium iodide or 3′,5′-Bis(hexadecyloxycarbonyl)-1′,4′-dihydro-1,2′,6′-trimethyl-3,4′-bipyridinium iodide (IUPAC name);

Derivative VIII (Example 2e)

1-Methyl-3-(2′,6′-dimethyl-3′,5′-dipropoxyethoxycarbonyl-1′,4′-dihydropyridyl-4′)-pyridinium iodide or 1′,4′,-Dihydro-1′,2′,6′-trimethyl-3′,5′-bis[(2-propoxyethoxy)carbonyl]-3,4′-bipyridinium iodide (IUPAC name);

Derivative IX (Example 2n)

1-Methyl-3-(2′,6′-dimethyl-3′,5′-di(2,3-dipentadecyloxycarbonyl)-propyloxycarbonyl-1′,4′-dihydropyridyl-4′)-pyridinium iodide or 3′,5′-Bis [(2,3-dipalmitoyloxypropoxy) carbonyl]-1′,4′-dihydro-1,2′,6′-trimethyl-3,4′-bipyridinium iodide (IUPAC name);

Derivative X (Example 2o)

1-Methyl-3-(2′,6′-dimethyl-3′,5′-dimenthyloxycarbonyl-1′,4′-di hydropyridyl-4′)-pyridinium iodide or 1′,4′-Dihydro-3′,5′-bis(menthyloxycarbonyl)-1,2′,6′-trimethyl-3,4′-bipyridinium iodide (IUPAC name);

Derivative XI (Example 2s)

1-Methyl-3-(2′,6′-dimethyl-3′,5′-dibornyloxycarbonyl-1′, 4′-dihydropyridyl-4′)-pyridinium iodide or 3′,5′-Bis(bornyloxycarbonyl)-1′,4′-dihydro-1,2′,6′-trimethyl-3,4′-bipyridinium iodide (IUPAC name);

Derivative XII (example 2r)

1-Methyl-3-(2′,6′-dimethyl-3′,5′-dicholesteryloxycarbonyl-1′,4′-dihydropyridyl-4′)-pyridinium iodide or 3′,5′-Bis(cholesteryloxycarbonyl)-1′,4′-dihydro-1,2′,6′-trimethyl-3,4′-bipyridinium iodide (IUPAC name);

Derivative XIII (Example 6a)

1-Nonyl-3-(2′,6′-dimethyl-3′,5′-diethoxycarbonyl-1′,4′-dihydropyridyl-4′)-pyridinium bromide or 3′,5′-Bis(ethoxycarbonyl)-1′,4′-dihydro-2′,6′-dimethyl-1-nonyl-3,4′-bipyridinium bromide (IUPAC name);

Derivative XIV (Example 6b)

1-Nonyl-3-(2′,6″-dimethyl-3,5″-ditetradecyloxycarbonyl-1′,4′-dihydropyridyl-4′)-pyridinium bromide or 1′,4′-Dihydro-2′,6′-dimethyl-1-nonyl-3′,5′-bis(tetradecyloxycarbonyl)-3,4′-bipyridinium bromide (IUPAC name);

Derivative XV (Example 8a)

1-Hexadecyl-4-(2′,6′-dimethyl-3′,5′-dietoxycarbonyl-1′,4′-dihydropyridyl-4′)-pyridinium bromide or 3′,5′-Bis(ethoxycarbonyl)-1-hexadecyl-1′,4′-dihydro-2′,6′-dimethyl-3,4′-bipyridinium bromide (IUPAC name);

Derivative XVI (Example 8d)

1-Hexadecyl-3-(2′,6′-dimethyl-3′,5′-dipropoxyethoxycarbonyl-1′,4′-dihydropyridyl-4′)-pyridinium bromide or 1-Hexadecyl-1′,4′-dihydro-2′,6′-dimethyl-3′,5′-bis[(2-propoxyethoxy)carbonyl]-3,4′-bipyridinium bromide (IUPAC name);

Derivative XVII (Example 8c)

1-Hexadecyl-3-(2′,6′-dimethyl-3′,5′-ditetradecyloxycarbonyl-1′, 4′-dihydropyridyl-4′)-pyridinium bromide or 1-Hexadecyl-1′,4′-dihydro-2′,6′-dimethyl-3′,5′-bis(tetradecyloxycarbonyl)-3,4′-bipyridinium bromide (IUPAC name);

Derivative XVIII (Example 8g)

1-Hexadecyl-3-(2′,6′-dimethyl-3′,5-dimenthyloxycarbonyl-1′,4′-dihydropyridyl-4′)-pyridinium bromide or 1-Hexadecyl-1′,4′-dihydro-3′,5′-bis(menthyloxycarbonyl)-2′,6′-dimethyl-3,4′-bipyridinium bromide (IUPAC name);

Derivative XIX (Example 3a)

1-Propyl-3-(2′,6′-dimethyl-3′,5′-dipropoxyethoxycarbonyl-1′,4′-dihydropyridyl-4′)-pyridinium iodide or 1′,4′-Dihydro-2′,6′-dimethyl-3′,5′-bis[(2-propoxyethoxy)carbonyl]-1-propyl-3,4′-bipyridinium iodide (IUPAC name);

Derivative XX (Example 11b)

1-Carbamoylmethyl-3-(2′,6′-dimethyl-3′,5′-dihexadecyloxycarbonyl-1′,4′-dihydropyridyl-4′)-pyridinium iodide or 1-Carbamoylmethyl-3′,5′-bis(hexadecyloxycarbonyl)-1′,4′-dihydro-2′,6′-dimethyl-3,4′-bipyridinium iodide (IUPAC name);

Derivative XXI (Example 4a)

1-Butyl-3-(2′,6′-dimethyl-3′,5′-ditetradecyloxycarbonyl-1′,4′-dihydropyridyl-4′-pyridinium bromide or 1-Butyl-1′,4′-dihydro-2′,6′-dimethyl-3′,5′-bis(tetradecyloxycarbonyl)-3,4′-bipyridinium bromide (IUPAC name);

Derivative XXII (Example 22a)

1,1′-[(3,5-Didecyloxycarbonyl-4-phenyl-1,4-dihydropyridine-2,6-diyldimethylene]bispyridinium dibromide or 1,1′-{[3,5-Bis(decyloxycarbonyl)-1,4-dihydro-4-phenylpyridine-2,6-diyl}dimethylene]bispyridinium dibromide (IUPAC name);

Derivative XXIII (Example 22b)

111-[(3,5-Didodecyloxycarbonyl-4-phenyl-14-dihydropyridine-2,6-diyl)dimethylene]bispyridinium dibromide or 1,1-{[3,5-Bis(dodecyloxycarbonyl)-1,4-dihydro-4-phenylpyridine-2,6-diyl]dimethylene}bispyridinium dibromide IUPAC name);

Derivative XXIV (Example 22c)

1,1-[(3,5-Ditetradecyloxycarbonyl-4-phenyl-1,4-dihydropyridine-2,6-diyl)dimethylene]bispyridinium dibromide or 1,1′-{[1,4-Dihydro-4-phenyl-3,5-bis(tetradecyloxycarbonyl)pyridine-2,6-diyl]dimethylene}bispyridinium dibromide (IUPAC name);

Derivative XXV (Example 22d)

1,1′-[(3,5-Dihexadecyloxycarbonyl-4-phenyl-1,4-dihydropyridine-2,6-diyl)dimethylene]bispyridinium dibromide or 1,1′-{[3,5-Bis(hexadecyloxycarbonyl)-1,4-dihydro-4-phenylpyridine-2,6-diyl]dimethylenelbispyridinium dibromide (IUPAC name);

Derivative XXVI (Example 18)

1-Hexadecyl-3-[2′,6′-dimethyl-3′, 5′-di(ethylthio)carbonyl-1′,4′-dihydropyridyl-4′]-pyridinium bromide or 3′,5′-Bis(ethylthiocarbonyl)-1-hexadecyl-1′,4′-dihydro-2′,6′-dimethyl-3,4′-bipyridinium bromide (IUPAC name);

Derivative XXVII (Example 23a)

N,N′-[(3,5-Didecyloxycarbonyl-4-phenyl-1,4-dihydropyridine-2,6-diyl)dimethylene]bis-N,N-dimethyloctylammoniumdibromide or N,N-{[3,5-Bis(decyloxycarbonyl)-1,4-dihydro-4-phenyl-pyridine-2,6-diyl]dimethylene}-N,N,N,N-tetramethyl-N,N-dioctyldiammonium dibromide (IUPAC name);

Derivative from Example 23b

N,N′-[(4-(2-Difluoromethoxyphenyl)-3,5-dimethoxycarbonyl-1,4-dihydropyridine-2,6-diyl)dimethylene]bistriethylammonium) dibromide or N,N-{{4-[2-(Difluoromethoxy)phenyl]-1,4-dihydro-3,5-bis(methoxycarbonyl)-pyridine-2,6-diyl}dimethylene}-N,N,N,N,N,N-hexaethyldiammonium dibromide (IUPAC name);

Derivative from Example 22f

1,1′-[(4-Difluoromethoxyphenyl-3,5-dimethoxycarbonyl-1-methyl-1,4-dihydropyridine-2,6-diyl)dimethylene]bispyridium dibromide or 1,1′-{{4-[2-(Difluoromethoxy)phenyl]-1,4-dihydro-3,5-bis(methoxycarbonyl)-1-methylpyridine-2,6-diyldimethylene}bispyridinium dibromide (IUPAC name);

Derivative from Example 19

2-Carbamoylmethylthio-3-cyano-5-[(N-ethoxycarbonylmethyl)-4-pyridyl]-6-methyl-4-(3-nitrophenyl)-1,4-dihydropyridine bromide or 6-Carbamoylmethylthio-5-cyano-1-ethoxycarbonylmethyl-1,4-dihydro-2-methyl-4-(3-nitrophenyl)-3,4-bipyridinium bromide (IUPAC name);

Derivative from Example 20a

6-[(N-Ethoxycarbonylmethyl)-4-pyridyl]-5-methyl-7-(3-nitrophenyl)-3-oxo-2,3-dihydro-7H-thiazolo[3,2-a]pyridine-8-carbonyltrile bromide or 4-(8-Cyano-5-methyl-7-(3-nitrophenyl)-3-oxo-2,3-dihydro-7H-thiazolo[3,2-a]pyridin-6-yl)-1-(ethoxycarbonylmethyl)pyridinium bromide (IUPAC name);

Derivative from Example 24a

1-Hexadecyl-3-{3-(1′-adamanthyloxycarbonyl)-1,4-dihydrobenzothieno[3,2-b]-pyridyl-5,5 dioxide-4}-pyridinium bromide or 3-[3-(1-Adamantyloxycarbonyl)-2-methyl-5,5-dioxo-4,5-dihydro-1H-benzo[4,5]thieno[3,2-b]pyridin-4-yl]-1-hexadecylpyridinium bromide (IUPAC name);

Derivative from Example 20b

N,N′-[(-2,6-Dimethyl-4-o-methoxyphenyl-1,4-dihydropyridine-3,5-diyl)ethoxycarbonyl]bis-N,N-dimethyloctylammoniumdiiodide or N,N′-{[1,4-Dihydro-4-(2-methoxyphenyl)-2,6-dimethylpyridine-3,5-diyl]bis(carbonyloxyethylene)}-N,N,N,N-tetramethyl-N,N-dioctyldiammonium diiodide (IUPAC name);

Derivative from Example 22e

1,1′[(3,5-Dioctadec-9′-enyloxycarbonyl-4-phenyl-1,4-dihydropyridine-2,6-diyl)dimethylene]bispyridiniumdibromide or 1,1′-{[1,4-Dihydro-3,5-bis(octadec-β-enyloxycarbonyl)-4-phenylpyridine-2,6-diyl]dimethylene}bispyridinium dibromide (IUPAC name).

Other potentially useful derivatives with the desirable properties are listed below: 1,1′-[(3,5-Didodecyloxycarbonyl-4-phenyl-1-methyl-1,4-dihydropyridine-2,6-diyl)dimethylene]bispyridinium dibromide; 1,1′-[(3,5-Didodecyloxycarbonyl-4-phenyl-1-hexyl-1,4-dihydropyridine-2,6-diyl)dimethylene]bispyridinium dibromide; 1,1′-[(3,5-Dihexadecylaminocarbonyl-4-phenyl-1-hexyl-1,4-dihydropyridine-2,6-diyl)dimethylene]bispyridinium dibromide; 1,1′-[(3,5-Di-N,N-dimethyloctylammonioethoxycarbonyl-4-phenyl-1,4-dihydropyridine-2,6-diyl)dimethylene]bispyridinium tetrabromide; 1,1′-[(2,6-Dimethyl-4-phenyl-1-methyl-1,4-dihydropyridine-3,5-diyl)ethoxycarbonyl]bispyridinium diiodide; 1,1′-[(3,5-Didodecyloxycarbonyl-4-ethoxycarbonyl-1,4-dihydropyridine-2,6-diyl)dimethylenelbispyridinium dibromide; 1,1′-[(4-Alkoxycarbonyl-3,5-didodecyloxycarbonyl-1,4-dihydropyridine-2,6-diyl)dimethylene]bispyridinium dibromide; 1,1′-[(4-Alkylamidocarbonyl-3,5-didodecyloxycarbonyl-1,4-dihydropyridine-2,6-diyl)dimethylene)bispyridinium dibromide; 4-Alkoxycarbonylmethyl-(3,5-didodecyloxycarbonyl-2,6-dipyridiniomethyl-1,4-dihydropyridine-2,6-diyl)dimethylene]bispyridinium dibromide; 1-Alkylamidocarbonyl-(3,5-didodecyloxycarbonyl-2,6-dipyridiniomethyl-1,4-dihydropyridyl-4)pyridinium tribromide; 1-Ethylamidocarbonylmethyl-3-(3,5-didodecyloxycarbonyl-2,6-dihydropyridiniomethyl-1,4-dihydropyridyl-4)pyridinium tribromide; 1,1′[(3,5-Diethoxycarbonyl-1-phenyl-4-phenyl-1,4-dihydropyridine-2,6-diyl)dimethylene]bispyridiinium dibromide; 1,1′[(3,5-Didodecyloxycarbonyl-1-phenyl-4-phenyl-1,4-dihydropyridine-2,6-diyl)dimethylene]bispyridiinium dibromide; or with their corresponding IUPAC names 1,1′-{[3,5-Bis(dodecyloxycarbonyl)-1-hexyl-1,4-dihydro-4-phenylpyridine-2,6-diyl]di-methylene}bispyridinium dibromide; 1,1′-{[3,5-Bis(hexadecyloxycarbonyl)-1-hexyl-1,4-dihydro-4-phenylpyridine-2,6-diyl]di-methylene}bispyridinium dibromide; 1,1′-({3,5-Bis[2-(dimethyloctylammonio)ethoxycarbony 1]-1,4-dihydro-4-phenylpyridine-2,6-diyl}dimethylene}bispyridinium tetrabromide; 1,1′-[(1,4-Dihydro-1,2,6-trimethyl-4-phenylpyridine-3,5-diyl)-bis(carbonyloxyethylene)]bispyridinium diiodide; 1,1′-{[3,5-Bis(dodecyloxycarbonyl)-4-ethoxycarbonyl-1,4-dihydropyridine-2,6-diyl]di-methylene}bispyridin ium dibromide; 1,1′-{[4-Alkoxycarbonyl-3,5-bis(dodecyloxycarbonyl)-1,4-dihydropyridine-2,6-diyl]di-methylene}bispyridin ium dibromides; 1,1′-{[4-Alkylcarbamoyl-3,5-bis(dodecyloxycarbonyl)-1,4-dihydropyridine-2,6-diyl]di-methylene}bispyridinium dibromides; 1,1′-([4-(Alkoxycarbonylmethyl)-3,5-bis(dodecyloxycarbonyl)-1,4-dihydropyridine-2,6-diyl]dimethylene}bis pyridinium dibromides; 3,5-Bis(dodecyloxycarbonyl)-1-ethylcarbamoylmethyl-1,4-dihydro-2,6-bis(1-pyri-diniomethyl)-3,4-bipyridiniumtribromide; 1-Alkylcarbamoylmethyl-3,5-bis(dodecyloxycarbonyl)-1,4-dihydro-2,6-bis (1-pyridiniomethyl)-3,4-bipyridinium tribromides; 1,1′-{[3,5-Bis(ethoxycarbonyl)-1,4-dihydro-1,4-diphenylpyridine-2,6-diyl]dimethylene}bispyridinium dibromide; 1,1′-{[3,5-Bis(dodecyloxycarbonyl)-1,4-dihydro-1,4-diphenylpyridine-2,6-diyl]dimethy-lene}bispyridinium dibromide.

The derivatives with the best DNA condensing properties are listed below:

Derivative I

1-Methyl-3-(2,6′-dimethyl-3′,5′-dipentadecyloxycarbonylethoxycarbonyl-1′,4′-dihydropyridyl-4′)-pyridinium iodide or 1′,4′-Dihydro-1,2′,6′-trimethyl-3′,5′-bis[(2-palmitoyloxyethoxy)carbonyl]-3,4′-bipyridinium iodide (IUPAC name);

Derivative V

1-Methyl-3-(2′,6′-dimethyl-3′,5′-didodecyloxycarbonyl-1′,4′-dihydropyridyl-4′)-pyridinium iodide or 3′,5′-Bis(dodecyloxycarbonyl)-1′,4′-dihydro-1,2′,6′-trimethyl-3,4′-bipyridinium iodide (IUPAC name);

Derivative VI

1-Methyl-3-(2′,6′-dimethyl-3′,5′-ditetradecyloxycarbonyl-1′,4′-dihydropyridyl-4′)-pyridinium iodide or 1′,4′-Dihydro-1,2′,6′-trimethyl-3′,5′-bis(tetradecyloxycarbonyl)-3,4′-bipyridinium iodide (IUPAC name);

Derivative XX

1-Carbamoylmethyl-3-(2′,6′-dimethyl-3′,5′-dihexadecyloxycarbonyl-1′,4′,-dihydropyridyl-4′)-pyridinium iodide or 1-Carbamoylmethyl-3′,5′-bis(hexadecyloxycarbonyl)-1,4′-dihydro-2′,6′-dimethyl-3,4′-bipyridinium iodide (IUPAC name);

Derivative XXIII

1,1′-[(3,5-Didodecyloxycarbonyl-4-phenyl-1,4-dihydropyridine-2,6-diyl)dimethylene]bispyridinium dibromide or 1,1′-{[3,5-Bis(dodecyloxycarbonyl)-1,4-dihydro-4-phenylpyridine-2,6-diyl]dimethylene}bispyridinium dibromide (IUPAC name);

Derivative XXIV

1,1′-[(3,5-Ditetradecyloxycarbonyl-4-phenyl-1,4-dihydropyridine-2,6-diyl)dimethylene]bispyridinium dibromide or 1,1′-{[1,4-Dihydro-4-phenyl-3,5-bis(tetradecyloxycarbonyl)pyridine-2,6-diyl]dimethylenelbispyridinium dibromide (IUPAC name);

Derivative XXVII

N,N′-[(3,5-Didecyloxycarbonyl-4-phenyl-1,4-dihydropyridine-2,6-diyl)dimethylene]bis-N,N-dimethyloctylammonium dibromide or N,N-{[3, 5-Bis(decyloxycarbonyl)-1,4-dihydro-4-phenylpyridine-2,6-diyl]dimethylene}-N,N,N,N-tetramethyl-N,N-dioctyldiammonium dibromide (IUPAC name).

The 1,4-dihydropyridines of the present invention can be synthesized according to methods well known in the art, the general principles of which are disclosed below and which are described in more detail below in the Examples.

Derivatives A were obtained from the respective acetoacetic esters, 2-, 3- or 4-pyridinecarbaldehyde and ammonia as shown schematically below:

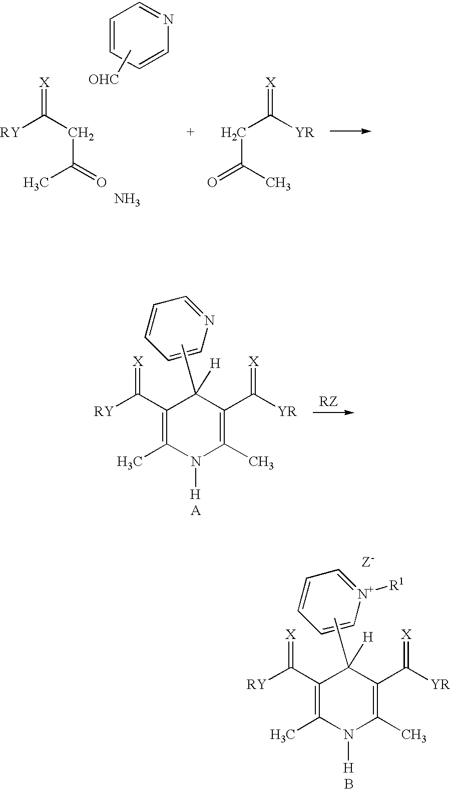

wherein

R is alkyl, substituted alkyl, aryl;

X is O, S, NR;

Y is C, S, NR″; R″═H, alkyl, aryl;

Z is a counter ion, preferably halogen;

R′ is alkyl, substituted alkyl, aryl:

wherein the number of C-atoms in the alkyl, acyl or aryl groups is as defined above.

When preparing the above compounds derivatives A are dissolvable e.g. by heating in acetone, methylethylketone or a mixture of acetone and chloroform, with subsequent addition or appropriate electrophilic agents. The product is subsequently refluxed and after cooling, the filtered precipitates are recrystallized.

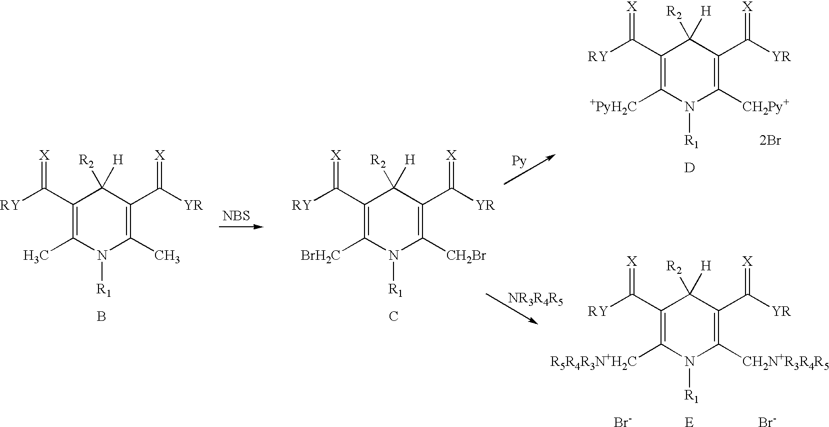

wherein

R is alkyl, substituted alkyl, aryl;

R₁ is H, alkyl, aryl, acyl;

R₂ is H, alkyl, substituted carbonyl, aryl, pyridinium, substituted pyridinium;

R₃, R₄,R₅ is alkyl;

X is O or S; and

Y is Of S or NH;

wherein the number of C-atoms is in alkyl, acyl or aryl groups is as define above.

Bromomethylderivatives C are obtainable by bromination of 1,4-dihydropyridines B with N-bromosuccinimide (NBS) in organic solvent, preferably methanol, at room temperature or at a lower temperature. Pyridinium (D) or ammonium (E) derivatives are obtainable by treating of bromomethylderivatives with pyridine or trialkylamine in appropriate organic solvent, preferably acetone or acetonitrile.

Applications of the Derivatives

In gene therapy, nucleotide containing compounds, such as nucleic acid, e.g. DNA, RNA, including other macromolecules, including proteins, polypeptides, antibodies and parts therof, as well as combinations of said nucleotides and/or polypeptides are used to produce, for example, a protein or a polypeptide, which has a desired effect on the disease to be treated. Gene transfer may result in stable or transient expression of the transferred gene by the cells. Gene therapy can be practiced either in vivo by direct gene transfer to the target cells in the body or ex vivo by gene transfer to cell cultures to be transplanted into the body. In both cases ability to transfer DNA in active form into cells is essential for success. After gene transfer, gene expression is regulated by the machinery of the cell and the regulatory elements of the transgene.

Depending on the gene sequence, gene expression may be limited to the cell population of interest or it may be induced with exogeneously administered compounds such as small molecular weight drugs. Successful gene transfer or introduction of exogeneous DNA into target cells is a prerequisite in gene therapy as well as in many other applications of gene technology. Transfer of nucleotide containing compounds such as DNA or RNA as well as chimeric DNA/RNA molecules or modified DNA or RNA, can be used to induce immunity by DNA vaccination. In this case it is also essential to transfer DNA in intact form into the target cells in the body either after injection or by application on mucosal surface.

Inhibitors of gene activity, such as antisense and antigene oligonucleotides and ribozymes are important forms of gene-based drugs. These compounds are composed of strands of DNA, RNA or their modified forms. They inhibit the function of the target gene either at the level of gene transcription, translation or splicing. Therefore, aberrant gene expression at too low or too high level or in wrong form can be corrected with these technologies.

Other forms of gene-based drugs include gene correction and gene modifier oligonucleotides. Gene correction oligonucleotides can for example be composed of chimeric oligonucleotide structures with modified RNA or DNA. These compounds are able to provide gene correction in the target cells at low frequency. Importantly, the gene correction is permanent and thus gene correction oligonucleotides can be used to treat genetic diseases. Gene modifier oligonucleotides are able to turn on or off gene expression e.g. at the level of gene promoter.

In all aforementioned forms of therapy or vaccination, DNA, RNA or their modified forms as such or as plasmids, vectors, etc., must be able to permeate into the cytoplasm or nucleus of the cells. Permeation is not optimal due to the hydrophilicity and the large molecular weights of these nucleotide containing compounds. They are also prone to degradation in body fluids and they bind to proteins in the cytoplasm of cells.

In addition to its medical applications gene transfer is an important part of modern research of cell biology, molecular biology and many other sub-disciplines of biology. Gene transfer is used frequently in laboratories in order to study the functions of particular gene sequences. Likewise transfer of antisense oligonucleotides is utilized to block the function of a certain gene and thereby elucidate its role in the cell biology. Importantly gene transfer is used in order to genetically engineer cells that express a certain gene in a stable fashion or under the control of a drug inducible gene promoter. Efficient gene transfer reagents are needed for the gene transfer protocols in the research laboratories.

Potential medical indications of the aforementioned technologies include a wide variety of genetic and acquired diseases based on disorders in gene expression. Examples of such diseases include cardiovascular diseases, neurological disorders, metabolic disorders, many disorders of the skin, eye, and lung. Gene therapy and administration of DNA, RNA or their modified forms may be practiced using different delivery or administration routes, including intravenous, oral, nasal, pulmonary, intramuscular, ocular, topical, subconjunctival, intravitreal, subretinal, dermal, topical, transdermal, electrically assisted or local application to different sites in the body e.g. during surgical inventions in liver, brain, tumor sites, blood vessels as an injection or a solid controlled release device or matrix, as microparticles or as implants.

Polyethylene glycol (PEG) may be mixed with the nucleotide containing 1,4-dihydropyridine derivatives complexes of the present invention to neutralize the surface of the liposome and to avoid the uptake of the complexes of the present invention by the liver and spleen after intravenous injection. Pegylated liposomes with a surface of PEG also reduce the interaction of the complexes with the proteins in the serum. Likewise ganglioside, hydroxypropyl methacrylate or sugar derivative containing lipids or polymers can be added to the formulations to modify the complex surface in such a way that the half-life of the complex is increased in the blood circulation. Furthermore, 1,4-dihydropyridine derivatives can be mixed with other lipids or polymers including PEI, DOGS, etc., that may modify the gene transfer properties. These include, fusogenic peptides and proteins, like proteins expressed by Haemophilus influenzae. Likewise, fusogenic lipids, like diolylphosphatidylethanolamine (DOPE), can be added to the complexes to facilitate the fusion of the complex with the endosomal wall of the cells. Furthermore, polymeric substances, like polyamidomine dendrimers and other dendritic structures, polyethylene imines (PEI) at various molecular sizes and shapes, poly-L-lysines, polymethyl methacrylates, polyhistidines, etc., can be used to make complexes with DNA and 1,4-dihydropyridine derivatives of the present invention.

The cationic liposomes from derivatives I-XXI (Table 2) can be prepared by dissolving the derivative in a suitable non-polar solvent, which is subsequently evaporated. The resulting thin films are resuspended e.g. in deionized water, vortexed and sonicated. To prepare the liposomes of derivatives I-XXI relatively high temperatures are used, e.g. in the range of 30-80° C., preferably 40-60° C., whereas derivatives XXI-XXVII are dissolvable in deionized water in ambient temperature. The methods for preparing the liposomes should be optimized separately for each derivative. The self-association properties and formation of liposomes in aqueous media can conveniently be studied by light scattering measurements.

Compounds XXI-XXVII can be formulated also in such a way that they are first dissolved in organic solvent with possible other lipids. The solvent is evaporated and the resulting thin lipid film is resuspended in water or buffer solution and sonicated.

Complexation of nucleotide containing compounds, such as DNA with the self-associating, liposome-forming 1,4-dihydropyridine derivatives can be demonstrated by using a gel mobility assay and/or a EtBr displacement test. Complexes of the 1,4-dihydropyridine derivative with the nucleotide containing compound(s) with different charge ratios can be prepared and are applicable for different delivery systems. Results obtained indicate that double-charged derivatives condense DNA more efficiently than single-charged derivatives.

In vitro transfer of nucleotide containing compounds are performable using cell cultures, the cells of which can be obtained from different sources and can be cultivated by per se known methods. The transfection efficiencies of the 1,4-dihydropyridine derivatives with nucleotide containing compounds at different charges can be evaluated by several per se known methods (Ruponen, M. et al., Biochem. Biophys. Acta, 1415 (1999), 331-341).

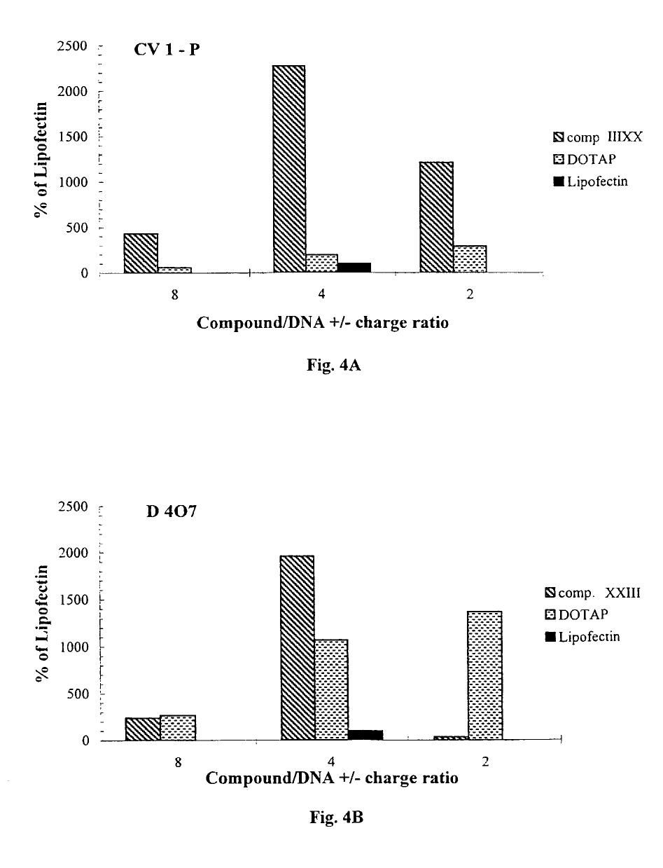

Results obtained in studies with in vitro gene transfection indicated that the transfection efficiencies were cell-line dependent. With both of the cell lines studied the double charged amphiphilic 1,4-dihydropyridine derivatives were more effective than the single-charged (Table 3). The transfection level of the amphiphilic derivative XXIII (Example 22b) observed with both cell lines examined, was at least twenty times higher than that of Lipofectin^{R} and with CV1-P cells ten times higher than transfection efficiency of DOTAP (FIG. 4).

In vivo gene transfer of nucleotide containing compounds was performed in animal models using XXIII/plasmid complexes. The transfer was recorded as marker gene expression in the arteries of the target organism. Previous tests with DOTMA/DOPE (Lipofectin^{R}) liposomes in the same animal model resulted in 0.05% gene transfer efficiency and were used as a reference when testing the 1,4-dihydropyridine derivatives of the present invention. The in vivo experiments performed, indicated that a charge ratio in the range of about +8-+1, preferably about +6-+2 is most effective. In said cases gene transfer efficiencies were between 0.05-1.5%.

When the cationic, amphiphllic 1,4-dihydropyridine derivatives of the present invention are complexed with nucleotide containing compounds and the complexes are used for treating diseases having a genetic component, including cancer or other inherited conditions, the 1,4-dihydropyridine derivatives of the present invention form liposomes, which are used as a part of the a therapeutic formulation in combination with other physiologically and/or pharmaceutically acceptable additives. The characteristics of the components in the delivery system depend on the route of administration. The delivery system formulation may contain, in addition to the nucleotide containing compound and the liposome-forming 1,4-dihydropyridine derivative, also other components, including lipids salts, buffers, stabilizers, solubilizers, and other materials well known in the art.

The preferred formulation used in the present invention may he nucleotide containing compounds combined with pharmaceutically acceptable additives and with the cationic, amphiphilic 1,4-dihydropyridine derivatives. These are generally provided as thin films, lamellar layers or liposomes. Administration of the nucleotide containing compounds with the liposome-forming 1,4-dihydropyridine derivatives can be carried out in a variety of conventional ways, such as by oral ingestion, inhalation, or cutaneous, subcutaneous, intramuscular, or intravenous injection.

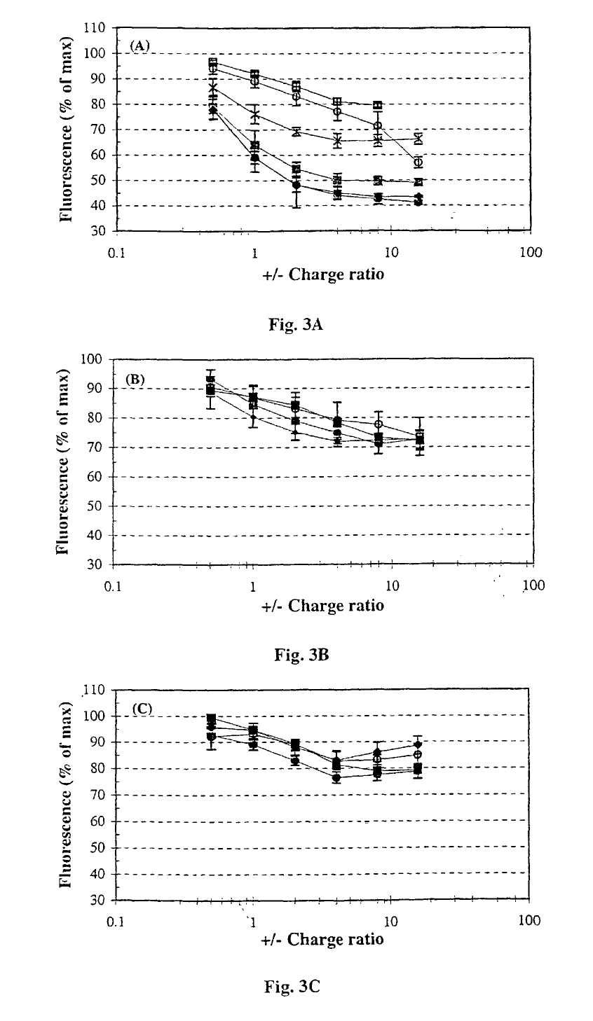

1,4-dihydropyridines can be formulated with DOPE. DOPE and 1,4-dihydropyridine are dissolved in organic solvent, which is evaporated to a thin film, which is then hydrated and sonicated to provide liposomes. The liposomes can be complexed with DNA. The complexes with DOPE condense DNA less efficiently than 1,4-dihydropyridine complexes as such (FIG. 3A-FIG. 3C). DOPE-containing 1,4-dihydropyridine complexes are able to transfer in a serum independent way (i.e. serum does not affect gene transfer). Therefore, DOPE containing complexes may be preferable, when complexes are in contact with serum, while the complexes without DOPE work best in serum-free conditions.

PEG modified or pegylated liposomes were also prepared using the thin film hydration method. Due to the surface shielding to more inert direction these complexes showed diminished transfection efficacy in vitro, but still the activity was significant and serum independent.

When administering formulations intravenously, cutaneously or subcutaneously they should be in form of a pyrogen-free, parenterally acceptable aqueous solution. The preparation of such parenterally acceptable solutions, having due regard to pH, isotonicity, stability, and the like, is within the skill in the art. A preferred formulation for intravenous, cutaneous, or subcutaneous injection could contain, in addition to the nucleotide containing compound and the 1,4-dihydropyridine derivative, an isotonic vehicle such as sodium chloride, Ringer's solution, dextrose or combination thereof. The delivery system formulation of the present invention may also contain stabilizers, preservatives, buffers, antioxidants, or other additives known to those of skill in the art.

In addition to the liquid formulations of the complexes the complexes can be dried using e.g. freeze drying, spray drying and other known methods to provide the complexes in powder form. These complexes can be reformulated into solid and semi-solid materials, such as controlled release polymers (leachable, bioerodible, biodegradable, channel forming). These preparations can be manufactured as matrices, reservoir devices, microspheres, or semi-solid pastes. Such technologies are well known in the state of art and they could provide controlled release of the DNA complexes containing 1,4-dihydropyridine derivatives over prolonged periods of time. Such devices could be placed in several tissues and body cavities to treat or prevent various diseases, since any DNA sequence can be complexed with 1,4-dihydropyridine derivatives and released with such systems.

The amounts of nucleotide containing compound and 1,4-dihydropyridine derivative in the formulation of the present invention and the duration of treatment will depend upon the nature and severity of the condition being treated, on the nature of prior treatments which the patient has undergone, and on the responses of the patient. Ultimately, the attending physician will decide the amounts of nucleotide containing compound and 1,4-dihydropyridine derivative with which to treat each individual patient and the duration of treatment. Initially, the attending physician will administer low doses of the formulation and observe the patient's response. Larger doses of the formulation are administered until the optimal therapeutic effect is obtained for the patient, and at that point the dosage is not increased further.

The derivatives and the liposome-forming compositions of the present invention as well as their preparation and their properties are described in more detail in the following examples. These examples are only illustrative and should not be interpreted as limiting the scope of the invention.

### EXAMPLE 1

Derivatives of 4-(2- or 3- or 4-pyridyl)-2,6-dimethyl-3,5-dialkoxycarbonyl-1,4-dihydropyridine

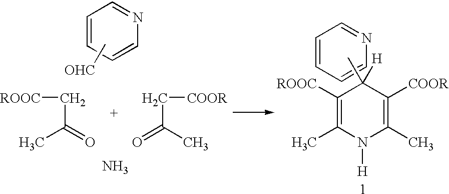

Derivatives 1a-1l were synthesized by a common procedure, whereby 0.05 mole of the corresponding pyridine aldehyde and 0.10 mole of the corresponding ester of acetoacetic acid are dissolved in 10-20 ml of ethanol, and 0.06 mole of 25% aqueous ammonia solution is added. The reaction mixture was refluxed for 3-7 h. Reaction is monitored by thin layer chromatography. After cooling, precipitate is filtered off, dried in air, and recrystallized from ethanol or ethanol water mixture.

Example 1a: R═(CH₂)₃CH(CH₃)₂, 4-β-Py;

Di(4-methylpenthyl)-1′,4′-dihydro-2′,6′-dimethyl-3,4′-bipyridine-3′,5′-dicarboxylate;

The yield is 67%, melting point 148-152° C.

Example 1b: R═CH₂C₆H₄—NO₂-p, 4-β-Py;

Di(4-nitrobenzyl)-1′,4′-dihydro-2′,6′-dimethyl-3,4′-dipyridine-3′,5′-dicarboxylate;

The yield is 51%, melting point 203-204° C.

Example 1c: R═(CH₂)₃OCC₁₆H₃₃, 4-β-Py;

Di(3-hexadecyloxypropyl)-1′,4′-dihydro-2′,6′-dimethyl-3,4′-bipyridine-3′,5′-dicarboxylate;

The yield is 78%, melting point 100-103° C.

Example 1d: R═(CH₂)₂OCOC₁₅H₃₁, 4-β-Py;

Di(2-palmitoyloxyethyl)-1′,4′-dihydro-2′,6′-dimethyl-3,4′-bipyridine-3′,5′-dicarboxylate;

The yield is 48%, melting point 100-1030C.

Example 1e: R═(CH₂)₃OCOC₁₅H₃₁, 4-β-Py;

Di(3-palmitoyloxypropyl)-1′,4′-dihydro-2′,6′-dimethyl-3,4′-bipyridine-3′,5′-dicarboxylate;

The yield is 75%, melting point 99-101° C.

Example 1f: R═CH₂CH(OCOC₁₅H₃₁)CH₂(OCOC₁₅H₃₁), 4-β-Py;

Di(2,3-dipalmitoyloxypropyl)-3,4′-bipyridine-3′,5′-dicarboxylate;

The yield is 65%, melting point 65-66° C.

Example 1g: R═menthyl, 4-β-Py;

Dimenthyl-1′,4′-dihydro-2′,6′-dimethyl-3,4′-bipyridine-3′,5′-dicarboxylate;

The yield is 57%, melting point 109-112° C.

Example 1h: R=1-Ad, 4-β-Py;

Di(1-adamanthyl)-1′,4′-dihydro-2′,6′-dimethyl-3,4′-bipyridine-3′,5′-dicarboxylate;

The yield is 45%, melting point 243-245° C.

Example 1i: R=cholesteryl, 4-α-Py;

Dicholesteryl-1′,4-dihydro-2′,6′-dimethyl-3,4′-bipyridine-3′,5′-dicarboxylate;

The yield is 62%, melting point 200-205° C. (decomp.).

Example 1j: R=bornyl, 4-β-Py;

Dibornyl-1′,4′-dihydro-2′,6′-dimethyl-3,4′-bipyridine-3′,5′-bicarboxylate;

The yield is 67%, melting point 240-243° C.

Example 1k: R=iso-bornyl, 4-β-Py;

Diisobornyl-1′,4,-dihydro-2′,6′-dimethyl-3,4′-bipyridine-3′,5′-dicarboxylate;

The yield is 51%, melting point 240-243° C.

Example 1l: R=bornyl, 4-γ-Py;

Dibornyl-1′,4′-dihydro-2′,6′-dimethyl-4,4′-bipyridine-3′,5′-dicarboxylate.

The yield is 47%, melting point 150-1520C.

### EXAMPLE 2

1-Methyl-(2- or 3- or 4-)(2′,6′-dimethyl-3′,5′-dialkoxycarbonyl-1′,4′-dihydropyridyl-4′)-pyridinium iodides

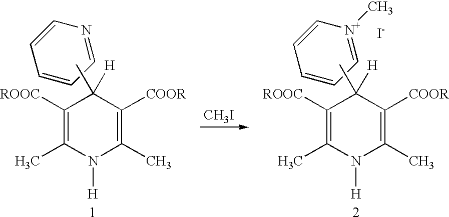

0.003 mole of the corresponding 4-(2- or 3- or 4- pyridyl)-2,6-dimethyl-3,5-dialkoxycarbonyl-1,4-dihydropyridine derivative was dissolved with heating in acetone or methylethylketone or a 1:1 mixture of acetone and chloroform and methyl iodide (1.3 ml, 2.2 g, 0.015 mole) was added two to three aliquots over 20 min. The product was refluxed for 1-3 h. After cooling, the filtered precipitate was recrystallized from acetone or methylethylketone.

Example 2a: R=n-C₄H₉, 4-β-Py;

3′,5′-Bis(butoxycarbonyl)-1′,4′-dihydro-1,2′,6′-trimethyl-3,4′-bipyridinium iodide;

The yield is 75%, melting point 161-164° C.

Example 2b: R=i-C₄H₉, 4-β-Py;

1′,4′-Dihydro-1,2′,6′-trimethyl-3′,5,-bis[(1-methylpropoxy)-carbonyl]-3,4′-bipyridinium iodide;

The yield is 66%, melting point 154-156° C.

Example 2c: R=t-C₄H₉, 4-β-Py;

3′,5′-Bis[(1,1-dimethylethoxy)carbonyl]-1′,4′-dihydro-1,2′,6′-trimethyl-3,4′-bipyridinium iodide;

The yield is 88%, melting point 166-169° C.

Example 2d: R═(CH₂)₃CH(CH₃)₂, 4-β-Py;

1′,4′-Dihydro-1,2′,6′-trimethyl-3′,5′-bis[(4-methylpenthyloxy)carbonyl]-3,4′-bipyridinium iodide;

The yield is 52%, melting point 97-100° C.

Example 2e (Derivative VIII): R═(CH₂)₂OC₃H₇, 4-β-Py;

1′,4′-Dihydro-1,2′,6′-trimethyl-3′,5′-bis[(2-propoxyethoxy)-carbonyl]-3,4′-bipyridinium iodide;

The yield is 88%, melting point 59-61° C.

Example 2f: R═CH₂C₆H₄—NO₂-p, 4-β-Py;

1′,4′-Dihydro-1,2′,6′-trimethyl-3′,5′-bis[(4-nitrobenzyloxy)-carbonyl]-3,4′-bipyridinium iodide;

The yield is 51%, melting point 202-203° C.

Example 2g (Derivative IV): R═C₉H₁₉, 4-β-Py;

1′,4′-Dihydro-1,2′,6′-trimethyl-3′,5′-bis(nonyloxycarbonyl)-3,4′-bipyridinium iodide;

The yield is 68%, melting point 95-960C (decomp.).

Example 2h (Derivative V): R═C₁₂H₂₅, 4-β-Py;

3′,5′-Dis(dodecyloxycarbonyl)-1′,4′-dihydro-1,2′,6′-trimethyl-3,4′-bipyridinium iodide;

The yield is 68%, melting point 108-111° C.

NMR data: ¹H NMR (CDCl₃): δ 0.85(t,6H, J=6 Hz, 3,5- . . . CH₃); 1.25-1.40(m,40H, 3,5- . . . (CH₂)₁₀); 2.60(s,6H, 2,6-CH₃); 4.01-(t,4H, J=6 Hz, 3,5-OCH₂); 4.65(s,3H, N—CH₃); 5.08(s,1H, 4-H); 7.34(b.s,1H, N—H); 7.84(d.d,1H, J_{5,4}=8 Hz, J_{5,6}=6 Hz, 5-H Py); 8.43(d, 1H, J_{4,5}=8 Hz, 4-H Py); 8.77(d,1H, J_{6,5}=6 Hz, 6-H Py); 8.95(s,1H, 2-H Py).

Example 2i (Derivative VI): R═C₁₄H₂₉, 4-β-Py;

1′,4′-Dihydro-1,2′,6′-trimethyl-3′,5′-bis(tetradecyloxycarbonyl)-3,4′-bipyridinium iodide;

The yield is 81%, melting point 114-115° C.

NMR data: ¹H NMR (CDCl₃): δ 0.87(t,6H, J=6 Hz, 3,5- . . . CH₃); 1.00-1.75(m,48H, 3,5- . . . (CH₂)₁₂); 2.50(s,6H, 2,6-CH₃); 4.00-(t,4H, J=6 Hz, 3,5-OCH₂); 4.63(s,3H, N—CH₃); 5.08(s,1H, 4-H); 7.52(b.s,1H, N—H); 7.83(d.d,1H, J_{5,4}=8 Hz, J_{5,6}=6 Hz, 5-H Py); 8.40(d, 1H, J_{4,5}=8 Hz, 4-H Py); 8.78(s,1H, 2-H Py); 8.90(d,1H, J_{6,5}=6 Hz, 6-H Py)

Example 2j (Derivative VII): R═C₁₆H₃₃, 4-β-Py;

3′,5′-Bis(hexadecyloxycarbonyl)-1′,4′-dihydro-1,2′,6′-trimethyl-3,4′-bipyridinium iodide;

The yield is 84%, melting point 112-113° C. (decomp.).

Example 2k (Derivative II); R═(CH₂)₃OC₁₆H₃₃, 4-β-Py;

3′,5′-Bis[(3-hexadecyloxypropoxy)carbonyl)]-1′,4′-dihydro-1,2′,6′-trimethyl-3,4′-bipyridinium iodide;

The yield is 91%, melting point 100-102° C.

Example 2l (Derivative I): R═(CH₂)₂OCOC₁₅H₃₁, 4-β-Py;

1′,4′-Dihydro-1,2′,6′-trimethyl-3′,5′-bis[(2-palmitoyloxyethoxy)carbonyl]-3,4′-bipyridinium iodide;

The yield is 81%, melting point 105-107° C.

NMR data: ¹H NMR (CDCl₃): δ 0.86(t,6H, J=6 Hz, 3,5- . . . CH₃); 1.00-1.78(m,52H, 3,5- . . . (CH₂)₁₃); 2.29(t,4H, J=7 Hz, 3,5-OCCH₂); 2.47(s,6H, 2,6-CH₃); 3.55-4.20(m,8H, 3,5-OCH₂CH₂O—); 4.60(s,3H, N—CH₃); 5.02(s,1H, 4-H); 7.70(b.s,1H, N—H); 7.86(d.d,1H, J_{5,4}=8 Hz, J_{5,6}=6 Hz, 5-H Py); 8.43(d,1H, J_{4,5}=8 Hz, 4-H Py); 8.82(s,1H, 2-H Py); 8.85(d,1H, J_{6,5}=6 Hz, 6-H Py).

Example 2m (Derivative III): R═(CH₂)₃OCOC₁₅H₃₁, 4-β-Py;

1′,4′-Dihydro-1,2′,6′-trimethyl-3′,5′-bis[(3-palmitoyloxypropoxy)carbonyl]-3,4′-bipyridinium iodide;

The yield is 94%, melting point 143-146° C.

Example 2n (Derivative IX): R═CH₂CH(OCOC₁₅H₃₁)CH₂(OCOC₁₅H₃₁),4-β-Py;

3-,5′-Bis (2,3-dipalmitoyloxypropoxy)carbonyl]-1′,4′-dihydro-1,2′,6′-trimethyl-3,4′-bipyridinium iodide;

The yield is 55%, melting point 75-770C.

Example 2o (Derivative X): R=menthyl, 4-β-Py;

1′,4′-Dihydro-3′,5′-bis(menthyloxycarbonyl)-1,2′,6′-trimethyl-3,4′-bipyridinium iodide;

The yield is 54%, melting point 156-159° C.

Example 2p (Derivative XI): R=1-Ad, 4-β-Py;

3′,5′-Bis[(1-adamanthyloxy)carbonyl]-1′,4′-dihydro-1,2′,6′-trimethyl-3,4′-bipyridinium iodide;

The yield is 77%, melting point 243-245° C.

Example 2r (Derivative XII): R=cholesteryl, 4-β-Py;

3′,5′-Bis(cholesteryloxycarbonyl)-1′,4′-dihydro-1,2′,6′-trimethyl-3,4′-bipyridinium iodide;

The yield is 71%, melting point 265° C. (decomp.).

Example 2s: R=bornyl, 4-β-Py;

3′,5′-Bis(bornyloxycarbonyl)-1′,4′-dihydro-1,2′,6′-trimethyl-3,4′-bipyridinium iodide;

The yield is 94%, melting point 271-274° C.

Example 2t: R=iso-bornyl, 4-β-Py;

3′,5′-Bis(isobornyloxycarbonyl)-1′,4′-dihydro-1,2′,6′-trimethyl-3,4′-bipyridinium iodide;

The yield is 72%, melting point 220-222° C.

Example 2u: R=bornyl, 4-γ-Py;

3′,5′-Bis(bornyloxycarbonyl)-1′,4′-dihydro-1,2′,6′-trimethyl-4,4′-bipyridinium iodide;

The yield is 64%, melting point 270° C. (decomp.).

Example 2v: R═C₂H₅, 4-α-Py;

3′,5′-Bis(ethoxycarbonyl)-1′,4′-dihydro-1,2′,6′-trimethyl-2,4′-bipyridinium iodide;

The yield is 75%, melting point 197-199° C.

### EXAMPLE 3

1-Propyl-(2- or 3- or 4-)(2′,6′-dimethyl-3′,5′-dialkoxycarbonyl-1′,4′-dihydropyridyl-4′)-pyridinium iodides

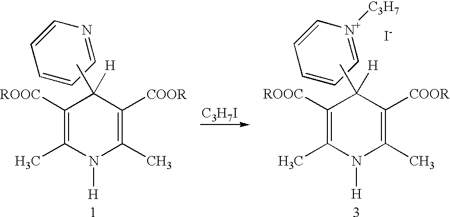

0.003 mole of the corresponding 4-(2- or 3- or 4-pyridyl)-2,6-dimethyl-3,5-dialkoxycarbonyl-1,4-dihydropyridine derivative were dissolved with heating in acetone and propyl iodide (1.47 ml, 2.57 g, 0.015 mole) was added. The product was refluxed for 8 h. After cooling, the filtered precipitate was recrystallized from acetone.

Example 3a (,Derivative XIX): R═(CH₂)20C₃H₇, 4-β-Py;

1′,4′-Dihydro-2′,6′-dimethyl-3′,5′-bis[(2-propoxyethoxy)carbonyl]-1-propyl-3,4′-bipyridinium iodide;

The yield is 79%, melting point 14′-142° C.

### EXAMPLE 4

1-Butyl-(2- or 3- or 4-) (2′,6′-dimethyl-3′,5′-dialkoxycarbonyl-1′,4′-dihydropyridyl-4′)-pyridinium bromides

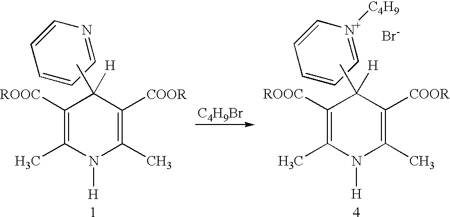

0.015 mole of the corresponding 4-(2- or 3- or 4-pyridyl) 2,6-dimethyl-3,5-dialkoxycarbonyl-1,4-dihydropyridine derivative were dissolved with heating in acetone and n-butyl bromide (4.83 ml, 6.17 g, 0.045 mole) was added. The product was refluxed for 40 h. After cooling, the filtered precipitate was recrystallized.

Example 4a (Derivative XXI): R═C₁₄H₂₉, 4-β-Py;

1-Butyl-1′,4′-dihydro-2′, 6′-dimethyl-3′,5′-bis(tetradecyloxycarbonyl)-3,4′-bipyridinium iodide;

The yield is 72%, melting point 86-87° C. (decomp.).

### EXAMPLE 5

1-Heptyl-(2- or 3- or 4-) (2′,6′-dimethyl-3′,5′-dialkoxycarbonyl-1′,4′-dihydropyridyl-4′)-pyridiniuin bromides

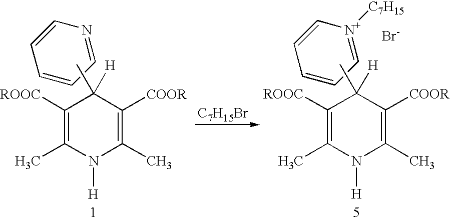

0.015 mole of the corresponding 4-(2- or 3- or 4-pyridyl)-2,6-dimethyl-3,5-dialkoxycarbonyl-1,4-dihydropyridin e derivative was dissolved with heating in methylethylketone and n-heptyl bromide (0.71 ml, 0.81 g, 0.015 mole) was added. The product was refluxed for 40 h. After cooling, the filtered precipitate was recrystallized.

Example 5a: R═C₂H₅, 4-β-Py;

3′,5′-Bis(ethoxycarbonyl)-1-heptyl-1′,4′-dihydro-2′,6′-dimethyl-3,4′-bipyridinium iodide;

The yield is 39%, melting point 165-167° C.

### EXAMPLE 6

1-Nonyl-(2- or 3- or 4-)(2′,6′-dimethyl-3′,5′-dialkoxycarbonyl-1′,4′-dihydropyridyl-4′)-pyridinium bromides

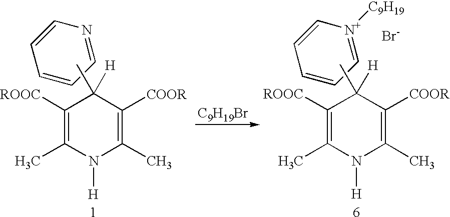

0.015 mole of the corresponding 4-(2- or 3- or 4-pyridyl)-2,6-di-methyl-3,5-dialkoxycarbonyl-1,4-dihydropyridine derivative was dissolved with heating in acetone and n-nonyl bromide (8.60 ml, 9.32 g, 0.045 mole) was added. The product was refluxed for 48 h. After cooling, the filtered precipitates was recrystallized.

Example 6a (Derivative XIII): R═C₂H₅, 4-β-Py;

3′,5′-Bis(ethoxycarbonyl)-1′,4′-dihydro-2′,6′-dimethyl-1-nonyl-3,4′-bipyridinium bromide;

The yield is 88%, melting point 142-143° C. (decomp.).

Example 6b (Derivative XIV): R═C₁₄H₂₉, 4-β-Py;

1′,4′-Dihydro-2′,6′-dimethyl-1-nonyl-3′,5′-bis(tetradecyloxycarbonyl)-3,4′-bipyridinium bromide;

The yield is 56%, melting point 124-126° C. (decomp.)

NMR data: ¹H NMR(CDCl₃): δ 0.87(overlap t,9H, 3,5- . . . CH₃+N— . . . —CH₃);1.22-202(m,62H,3,5- . . . (CH₂)₁₂+N— . . . (CH₂)₇);2.54(s,6H, 2,6-CH₃);4.00(t,4H,J=7 Hz, 3,5-OCH₂); 4.76(t,2H,J=7 Hz,N—CH₂); 5.08(s,1H, 4-H); 7.90 (d.d,1H, J_{5,4}=8 Hz, J_{5,6}=6 Hz, 5-H Py); 8.34(d,1H, J_{4,5}=8 Hz,4-H Py);8.62(b.s,1H,N—H);8.76(s,1H, 2-H Py) 9.25(d,1H, J_{6,5}=6 Hz, 6-H Py)

### EXAMPLE 7

1-Dodecyl-(2- or 3- or 4-)(2′,6′-dimethyl-3′,5′-dialkoxycarbonyl-1,4-dihydropyridyl-4)-pyridinium bromides

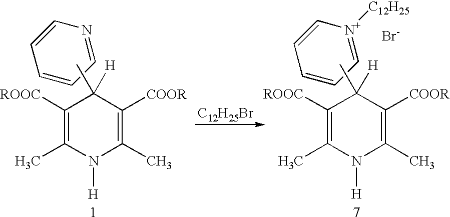

0.009 mole of the corresponding 4-(2- or 3- or 4-pyridyl) -2,6-dimethyl-3,5-dialkoxycarbonyl-1,4-dihydropyridine derivative was dissolved with heating in methylethylketone and n-dodecyl bromide (2.20 ml, 2.27 g, 0.009 mole) was added. The product was refluxed for 20 h. After cooling, the filtered precipitate was recrystallized.

Example 7a: R═C₂H₅, 4-β-Py;

1-Dodecyl-3′,5′-bis (ethoxycarbonyl)-1′, 4′-dihydro-2D,6-dimethyl-3′,5′-bipyridinium bromide;

The yield is 60%, melting point 162-164° C.

### EXAMPLE 8

1-Hexadecyl-(2- or 3- or 4-) (2′,6′-dimethyl-3′,5′-dialkoxycarbonyl-1′,4′-dihydropyridyl-4′)-pyridinium bromides

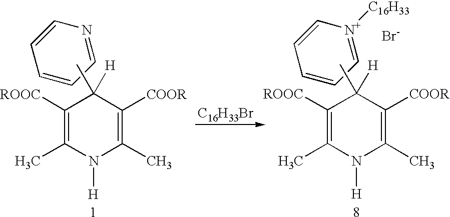

0.003 mole of the corresponding 4-(2- or 3- or 4-pyridyl)-2,6-dimethyl-3,5-dialkoxycarbonyl-1,4-dihydropyridine derivative was dissolved with heating in acetone or methylethylketone or a 1:1 mixture of acetone and chloroform and n-hexadecyl bromide (0.9 ml, 0.9 g, 0.003 mole) was added. The product was refluxed for 45-60 h. After cooling, the filtered precipitate was recrystallized from acetone or methylethylketone.

Example 8a (Derivative XV): R═C₂H₅, 4-β-Py;

3′,5′-Bis (ethoxycarbonyl)-1-hexadecyl-1′,4′-dihydro-2′,6′-dimethyl-3,4′-bipyridinium bromide;

The yield is 63%, melting point 135-136° C.

Example 8b: R=sec-C₄H₉, 4-β-Py;

1-Hexadecyl-1′,4′-dihydro-2′,6′-dimethyl-3′,5′-bis[(1-methyl-propoxy)carbonyl]-3,4′-bipyridinium bromide;

The yield is 41%, melting point 159-165° C.

Example 8c (Derivative XVII): R═C₁₄H₂₉; 4-β-Py;

1-Hexadecyl-1′,4′-dihydro-2′,6′-dimethyl-3′,5′-bis(tetradecyloxycarbonyl)-3,4′-bipyridinium bromide;

The yield is 89%, melting point 133-135° C.

Example 8d (Derivative XVI): R═(CH₂)₂OC₃H₇; 4-β-Py;

1-Hexadecyl-1′,4′-dihydro-2′,6′-dimethyl-3′,5′-bis[(2-propoxyethoxy)carbonyl]-3,4′-bipyridinium bromide;

The yield is 79%, melting point 93-96° C.

Example 8e: R═(CH₂)₃CH(CH₃)₂; 4-β-Py;

1-Hexadecyl-1,4′-dihydro-2,6′-dimethyl-3,5′-bis[(4-methylpenthyloxy)carbonyl]-3,4′-bipyridinium bromide;

The yield is 54%, melting point 107-110° C.

Example 8f: R═CH₂C₆H₄—NO₂-p, 4-β-Py;

1-Hexadecyl-1′,4′-dihydro-2′,6′-dimethyl-3′,5′-bis[(4-nitrobenzyloxy)carbonyl]-3,4′-bipyridinium bromide;

The yield is 65%, melting point 89-93° C.

Example 8g (Derivative XVIII): R=menthyl, 4-β-Py;

1-Hexadecyl-1′,4′-dihydro-3′,5′-bis(menthyloxycarbonyl)-2′,6′-dimethyl-3,4′-bipyridinium bromide;

The yield is 52%, melting point 115-117° C.

NMR data: ¹H NMR (CDCl₃): δ 0.55-2.14(m,65H, 3,5-menthyl+N— . . . (CH₂)₁₄CH₃);2.51(s,6H,2,6-CH₃);4.48-4.84(m,4H,3,5-OCH+N—CH₂); 5.05(s,1H, 4-H); 7.88(d.d, 1H, J_{5,4}=8 Hz, J_{5,6}=6 Hz, 5-H Py); 8.29(d,1H, J_{4,5}=8 Hz, 4-H Py); 8-57(b.s,1H, N—H);8.73 (s,1H, 2-H Py); 9.29(d,1H, J_{6,5}=6 Hz, 6-HPy).

Example 8h: R═C₂H₅, 4-γ-Py;

3′,5′-Bis(ethoxycarbonyl)-1-hexadecyl-1′,4′-dihydro-2′,6′-dimethyl-4,4′-bipyridinium bromide;

The yield is 63%, melting point 95-98° C.

Example 8i: R═C₂H₅, 4-α-Py;

3′,5′-Bis(ethoxycarbonyl)-1-hexadecyl-1′,4′-dihydro-2′,6′-dimethyl-2,4′-bipyridinium bromide;

The yield is 8%, melting point 117-121° C.

### EXAMPLE 9

1-Ethoxycarbonylmethyl-(2- or 3- or 4-)(2′,6′-dimethyl -3′,5′-dialkoxycarbonyl-1,4′-dihydropyridyl-4′)-pyridinium bromides

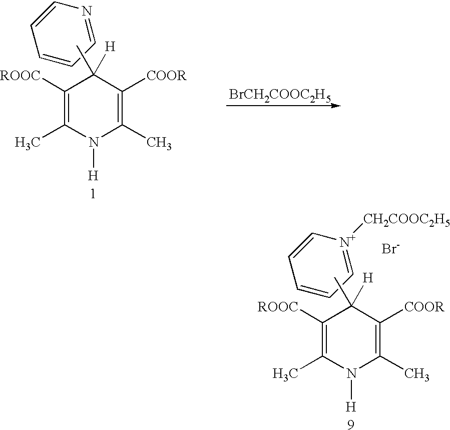

0.003 mole of the corresponding 4-(2- or 3- or 4-pyridyl)-2,6-dimethyl-3,5-dialkoxycarbonyl-1,4-dihydropyridine derivative was dissolved with heating in acetone or methylethylketone or a 1:1 mixture of acetone and chloroform and ethyl bromoacetate (0.32 ml, 0.5 g, 0.03 mole) was added. The product was refluxed for 2-9 h. After cooling, the filtered precipitate was recrystallized from acetone or methylethylketone or 1:1 mixture of ethanol and hexane.

Example 9a: R═CH₃, 4-β-Py;

1-(2-Ethoxy-2-oxoethyl)-1′,4′-dihydro-3′,5′-bis(methoxycarbonyl)-2′,6′-dimethyl-3,4-bipyridinium bromide;

The yield is 85%, melting point 180° C. (decomp.)

Example 9b: R═(CH₂)₂OC₂H₅, 4-β-Py;

3′,5′-Bis[(2-ethoxyethoxy)carbonyl]-1-(2-ethoxy-2-oxoethyl)-1′,4′-dihydro-2′,6′-dimethyl-3,4′-bipyridinium bromide;

The yield is 66%, melting point 163-165° C.

Example 9c: R═(CH₂)₂OC₃H₇, 4-β-Py;

1-(2-Ethoxy-2-oxoethyl)-1′,4′-dihydro-2′,6′-dimethyl-3′,5′-bis[(2-propoxyethoxy)carbonyl]-3,4′-bipyridinium bromide;

The yield is 91%, melting point 144-146° C.

Example 9d: R═C₁₆H₃₃, 4-β-Py;

1-(2-Ethoxy-2-oxoethyl)-3′,5′-bis(hexadecyloxycarbonyl)-1′,4′-dihydro-2′,6′-dimethyl-3,4′-bipyridinium bromide;

The yield is 89%, melting point 99-102° C.

Example 9e: R=menthyl, 4-β-Py;

1-(2-Ethoxy-2-oxoethyl)-1′,4′-dihydro-3′,5′-bis(menthyloxycarbonyl)-2′,6′-dimethyl-3,4′-bipyridinium bromide;

The yield is 81%, melting point 180° C. (decomp.)

Example 9f: R=1-Ad, 4-β-Py;

3′,5′-Bis(1-adamanthyloxycarbonyl)-1-(2-ethoxy-2-oxoethyl)-1′,4′-dihydro-2′,6′-dimethyl-3,4′-bipyridinium bromide;

The yield is 69%, melting point 220° C. (decomp.).

Example 9g: R═C₂H₅, 4-γ-Py;

3′,5′-Bis(ethoxycarbonyl)-1-(2-ethoxy-2-oxoethyl)-1′,4′-dihydro-2′,6′-dimethyl-4,4′-bipyridinium bromide;

The yield is 47%, melting point 140-143° C.

Example 9h: R═C₂H₅, 4-α-Py;

3′,5′-Bis(ethoxycarbonyl)-1-(2-ethoxy-2-oxoethyl)-1′,4′-dihydro-2′,6′-dimethyl-2,4′-bipyridinium bromide;

The yield is 34%, melting point 197-199° C.

### EXAMPLE 10

1-Phenacyl-(2- or 3- or 4-) (2′,6′-dimethyl-3′,5′-dialkoxycarbonyl-1′,4′-dihydropyridyl-4′)-pyridinium bromides

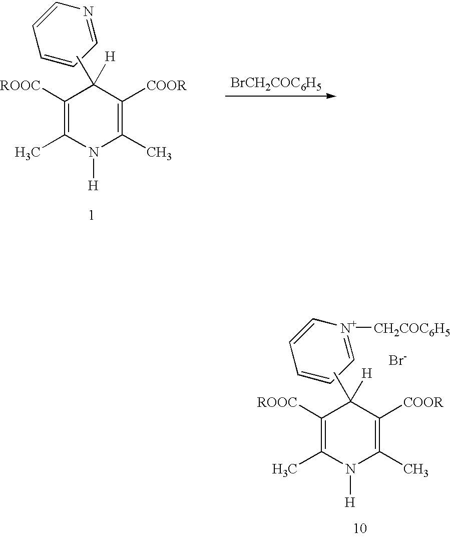

0.005 mole of the corresponding 4-(2- or 3- or 4-pyridyl)-2,6-dimethyl-3,5-dialkoxycarbonyl-1,4-dihydropyridine derivative was dissolved in acetone or methylethylketone and 2-bromoacetophenone (1.0 g, 0.005 mole) was added. The mixture was stirred at room temperature for 24 h. The precipitate was filtered off and recrystallized from acetone or methylethylketone.

Example 10a: R═(CH₂)₂OC₃H₇, 4-β-Py;

1′,4′-Dihydro-2′,6′-dimethyl-1-phenacyl-3′,5′-bis[(2-propoxyethoxy)carbonyl]-3,4′-bipyridinium bromide;

The yield is 67%, melting point 135-138° C.

Example 10b: R═C₁₆H₃₃, 4-β-PY;

1′,4′-Dihydro-3′, 5′-bis (hexadecyloxycarbonyl)-2′, 6′-dimethyl-1-phenacyl-3,4′-bipyridinium bromide;

The yield is 78%, melting point 148-150° C.

Example 10c: R═C₂H₅, 4-γ-Py;

3′, 5′-Bis (ethoxycarbonyl)-1′,4′-dihydro-2′,6′-dimethyl-1-phenacyl-4,4′-bipyridinium bromide,

The yield is 84%, melting point 224-227° C.

Example 10d: R═C₂H₅, 4-α-Py;

3′,5′-Bis(ethoxycarbonyl)-1′,4′-dihydro-2′,6′-dimethyl-1-phenacyl-2,4′-bi-vridinium bromide;

The yield is 46%, melting point 185-188° C.

### EXAMPLE 11

1-Carbamoylmethyl-(2- or 3- or 4-)(2′,6′-dimethyl-3′,5′-dialkoxycarbonyl-1′,4′-dihydropyridyl-4′)-pyridinium iodides

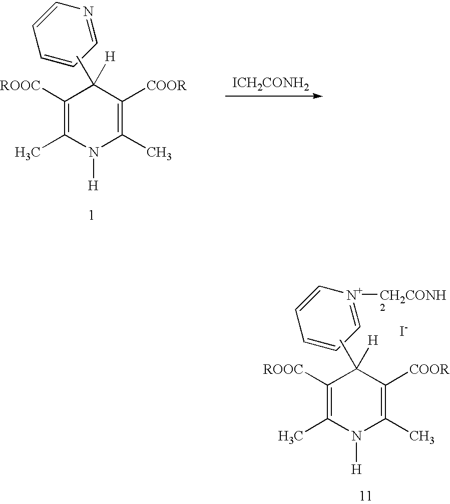

0.005 mole of the corresponding 4-(2- or 3- or 4-pyridyl)-2,6-dimethyl-3,5-dialkoxycarbonyl-1,4-dihydropyridine derivative was dissolved with heating in acetone or a 1:1 mixture of acetone and chloroform and iodoacetamide (1.0 g, 0.005 mole) was added. The mixture was refluxed for 3-5 h. After cooling, the filtered precipitate was recrystallized from ethanol or water.

Example 11a: R═(CH₂)₂OC₃H₇, 4-β-Py;

1-Carbamoylmethyl-1′,4′-dihydro-2′,6′-dimethyl-3′,5′-bis[(2-propoxyethoxy)carbonyl]-3,4′-bipyridinium iodide;

The yield is 50%, melting point 14′-143° C.

Example 11b: R═C₁₂H₂₅ 4-β-Py;

1-Carbamoylmethyl-3′,5′-bis(dodecyloxycarbonyl)-1′,4′-dihydro-2′,6′-dimethyl-3,4′-bipyridinium iodide;

The yield is 90%, melting point 151-1530C.

Example 11c (Derivative XX): R═C₁₆H₃₃, 4-β-Py;

1-Carbamoylmethyl-3′,5′-bis(hexadecyloxycarbonyl)-1′,4′-dihydro-2′,6′-dimethyl-3,4′-bipyridinium iodide;

The yield is 84%, melting point 140-141° C. (decomp.).

Example 11d: R=1-Ad, 4-β-Py;

3′,5′-Bis(1-adamanthyloxycarbonyl)-1-carbamoylmethyl-1′,4′-dihydro-2′,6′-dimethyl-3,4′-bipyridinium iodide;

The yield is 66%, melting point 190° C. (decomp.).

### EXAMPLE 12

1-(2-Naphthacyl)-(2- or 3- or 4-) (2′,6′-dimethyl-3′,5-dialkoxycarbonyl-1′,4′-dihydropyridyl-4′)-pyridinium bromides

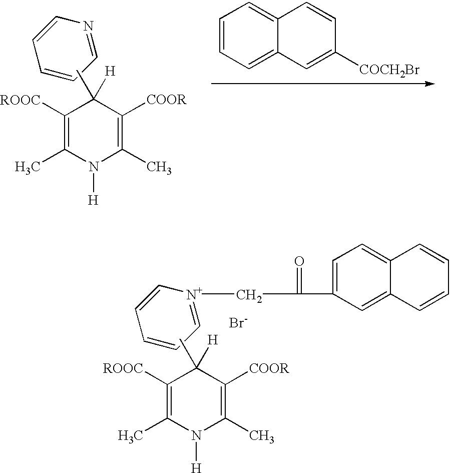

0.006 mole of the corresponding 4-(2- or 3- or 4-pyridyl)-2,6-dimethyl-3,5-dialkoxycarbonyl-1,4-dihydropyridine derivative was dissolved with heating in a 1:1 mixture of acetone and chloroform and 2-bromo-2-acetonaphthone (1.50 g, 0.006 mole) was added. The mixture was refluxed for 12-15 h. After cooling, the filtered precipitate was recrystallized from ethanol.

Example 12a: R═C₂H5, 4-β-Py;

3′,5′-Bis(ethoxycarbonyl)-1′,4,′-dihydro-2′,6′-dimethyl-1-(2-naphthoylmethyl)-3,4,-bipyridinium bromide;

The yield is 87%, melting point 236-240° C.

Example 12b: R═C₂H₅, 4-γ-Py;

3′,5′-Bis (ethoxycarbonyl)-1′,4′-dihydro-2′,6′-dimethyl-1-(2-naphthoylmethyl)-4,4′-bipyridinium bromide;

The yield is 34%, melting point 211-216° C.

### EXAMPLE 13

1-Carboxydecyl-(2- or 3- or 4-) (2′,6′-dimethyl-3′,5′-dialkoxycarbonyl-1,4-dihydropyridyl-4)-pyridinium bromides

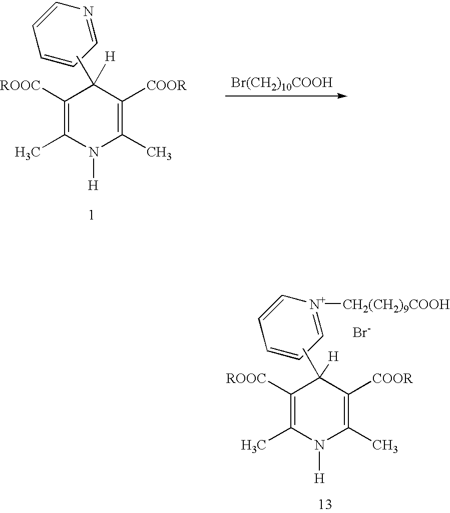

0.004 mole of the corresponding 4-(2- or 3- or 4- pyridyl) -2,6-dimethyl-3,5-dialkoxycarbonyl-1,4-dihydropyridine derivative was dissolved with heating in methylethylketone and 11-bromindecanoic acid (1.00 g, 0.004 mole) was added. The mixture was refluxed for 30 h. After cooling, the filtered precipitate was recrystallized.

Example 13a: R═C₂H₅, 4-β-Py;

1-(10-Carboxydecyl) -3′,5′-bis (ethoxycarbonyl)-1′,4′-dihydro-2′,6′-dimethyl-3,4′-bipyridinium bromide;

The yield is 43%, melting point 134-136° C.

Example 13b: R═O₂H₅, 4-γ-Py;

1-(10-Carboxydecyl) -3′,5′-bis (ethoxycarbonyl)-1′, 41′-dihydro-2′,6′-dimethyl-4,4′-bipyridinium bromide;

The yield is 77%, melting point—r.t.

### EXAMPLE 14

1-Carboxyundecyl-(2- or 3- or 4-)(2′,6′-dimethyl-3′,5′-dialkoxycarbonyl-1′,4′-dihydropyridyl-4 )-pyridinium bromides

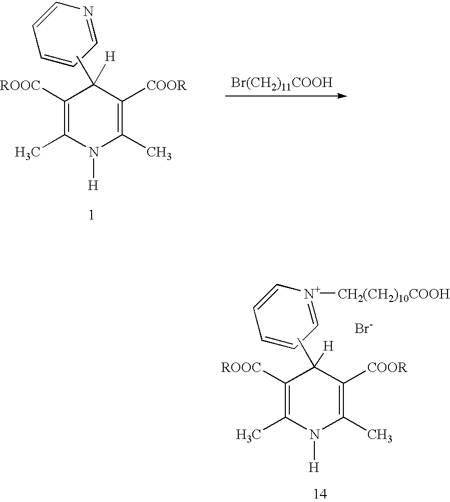

0.004 mole of the corresponding 4-(2- or 3- or 4- pyridyl)-2,6-dimethyl-3,5-dialkoxycarbonyl-1,4-dihydropyridine-derivative was dissolved with heating in acetone or methylethylketone and 12-bromdodecanoic acid (1.00 g, 0.004 mole) was added. The mixture was refluxed for 53 h. After cooling, the filtered precipitate was recrystallized.

Example 14a: R═C₂H₅, 4-β-Py;

1-(11-Carboxyundecyl)-3′,5′-bis(ethoxycarbonyl)-1′,4′-dihydro-2′,6′-dimethyl-3,4′-bipyridinium bromide;

The yield is 61%, melting point 152-156° C.

### EXAMPLE 15

1-(3″-Cholesteryloxycarbonyl-(4′″-butyl))-(2- or 3- or 4-) (2′,6′-dimethyl-3′,5′-dialkoxycarbonyl-1′,4′-dihydropyridy-1-4)-pyridinium bromides

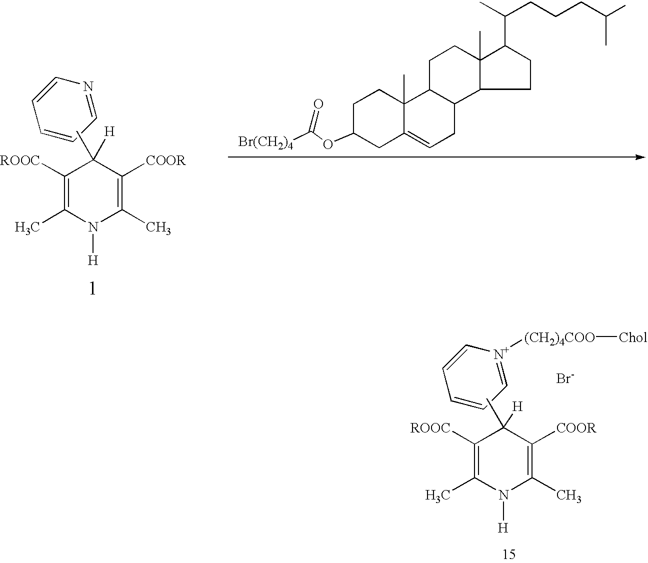

0.0011 mole of the corresponding 4-(2- or 3- or 4-pyridyl)-2,6-dimethyl-3,5-dialkoxycarbonyl-1,4-dihydropyridin e derivative was dissolved with heating in acetone and cholesteryl-5-bromovalerate (0.6 g, 0.011 mole) was added. The mixture was refluxed for 70 h. After cooling, the filtered precipitate was recrystallized.

Example 15a: R═C₂H₅, 4-β-Py;

1-[4-(3-Cholesteryloxycarbonyl)butyl]-3′,5′-bis(ethoxycarbonyl)-1,4′-dihydro-2′,6′-dimethyl-(2 or 3 or 4),4′-bipyridiniumbromide;

The yield is 48%, melting point 225-227° C.

### EXAMPLE 16

1-(4¹¹-Nitrobenzyl)-(2- or 3- or 4-)(2′,6′-dimethyl-3′,5′-dialkoxycarbonyl-1′,4′-dihydropyridyl-41) -pyridinium bromides

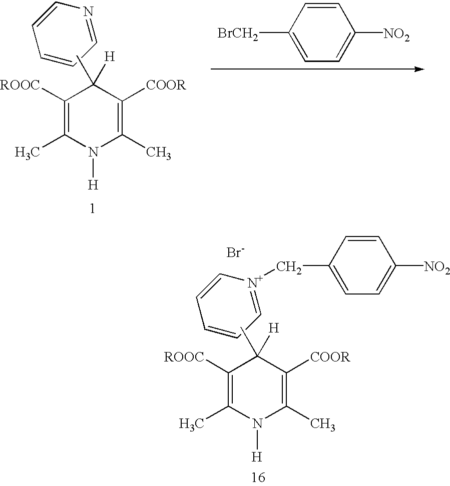

0.003 mole of the corresponding 4-(2- or 3- or 4-pyridyl)-2,6-dimethyl-3,5-dialkoxycarbonyl-1,4-dihydropyridine derivative was dissolved with heating in acetone and 4-nitrobenzyl bromide (0.65 g, 0.003 mole) was added. The mixture was refluxed for 23 h. After cooling, the filtered precipitates was recrystallized.

Example 16a: R═C₂H₅, 4-β-Py;

3′,5′-Bis(ethoxycarbonyl)-1′,4′-dihydro-2′,6′-dimethyl-1-(4-nitrobenzyl)-3,4′-bipyridinium bromide;

The yield is 73%, melting point 212-213° C. (decomp.).

### EXAMPLE 17

1-(2,4-Dinitrophenyl)-(2- or 3- or 4-)(2′,6′-dimethyl-3′,5′-di-alkoxycarbonyl-1′,4′-dihydropyridyl-4′)-pyridinium chlorides

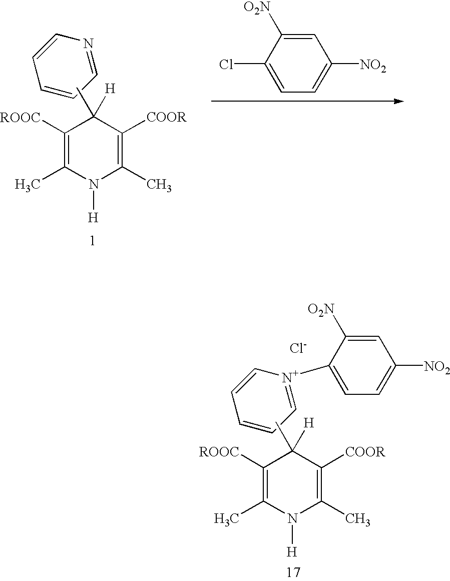

0.003 mole of the corresponding 4-(2- or 3- or 4-pyridyl)-2,6-dimethyl-3,5-dialkoxycarbonyl-1,4-dihydropyridine derivative was dissolved with heating in acetone and 1-chloro-2,4-dinitrobenzene (0.61 g, 0.003 mole) was added. The mixture was refluxed for 47 h. After cooling, the filtered precipitate was recrystallized.

Example 17a: R═C₂H₅, 4-β-Py;

3′,5′-Bis(ethoxycarbonyl)-1′, 4′-dihydro-2′,6′-dimethyl-1-(2,4-dinitrophenyl)-3,4′-bipyridinium chloride;

The yield is 70%, melting point 178-180° C.

### EXAMPLE 18

1-Hexadecyl-3-(2′,6′-dimethyl-3′,5′-di(ethylthio)-carbonyl-1′,4′-dihydropyridyl-4′)-pyridinium bromide

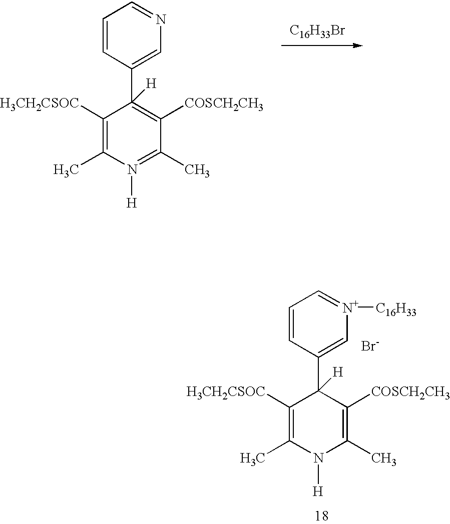

0.25 g (0.8 mmole) hexadecylbromide was added by stirring to a solution of 0.3 g (0.8 mmole) of 2,6-dimethyl-4-(3′-pyri-dyl)-3,5-di(ethylthio)carbonyl-1,4-dihydropyridine in 3 ml of anhydrous 2-butanone and the mixture was refluxed for 60 h. After cooling the yellow precipitate was filtered and crystallized from methanol. 0.5 g (91%) of II was obtained. Mp 119-121° C.

Example 18a:

3′,5′-Bis[(ethylthio)carbonyl]-1-hexadecyl-1′,4′-dihydro-2′,6′-dimethyl-3,4′-bipyridinium bromide.

Anal.Calcd. for C₃₄H₅₅BrN₂O₂S₂: C 61.15; H 8.30; N 4.19; S 9.60. Found: C 61.32; H 8.45; N 4.00; S 9.20.

### EXAMPLE 19

2-Carbamoylmethylthio-3-cyano-5-[(N-ethoxycarbonylmethyl)-4-pyridyl]-6-methyl-4-(3-nitrophenyl)-1,4-dihydropyridine bromide

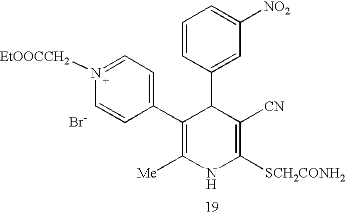

A mixture of 2-carbamoylmethylzhio-3-cyano-5-(4-pyridyl)-6-methyl-4-(3-nitrophenyl)-1,4-dihydropyridine (0.81 g, 2 mmol) and ethyl bromoacetate (0.66 ml, 6 mmol) in 10 ml of ethanol was refluxed for 15 min, hot filtered and cooled to 10° C. The precipitate was filtered, washed with cold ethanol (5 ml) to give 1.06 g (90%) of desired bromide as yellow crystals, mp 178-180° C.

Example 19a:

6-Carbamoylmethylthio-5-cyano-1i-ethoxycarbonylmethyl-1,4-dihydro-2-methyl-4- (3-nitrophenyl) -3,4′-bipyridinium bromide.

Elemental analysis: Found: C 49.96; H 4.45; N 12.03; S 5.44; Calcd. for C₂₄H₂₄BrN₅O₅S: C 50.18; H 4.21; N 12.19; S 5.58.

### EXAMPLE 20

3-(3,5)-pyridinio(trialkylammonio)-1,4-dihydropyridine derivatives

Example 20a

6-[(N-ethoxycarbonylmethyl)-4-pyridyl]-5-methyl-7-(3-nitrophenyl)-3-oxo-2,3-dihydro-7H-thiazolo[3,2-a]pyridine-8-carbonitrile bromide

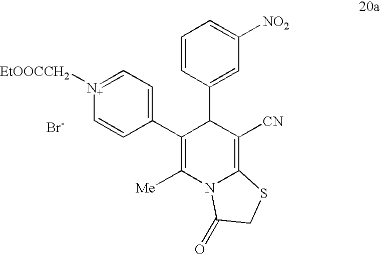

A mixture of 5-methyl-7-(3-nitrophenyl)-3-oxo-6-(4-pyridyl)-2,3-dihydro-7H-thiazolo[3,2-a]pyridine-8-carbonitrile (1.95 g, 5 mmol) and ethyl 2-bromoacetate (1.11 ml, 10 mmol) in 10 ml of ethanol and 5 ml of DMF was refluxed for 5 min, filtered and cooled to 0° C. The precipitate was filtered, washed with cold ethanol (5 ml) to give 2.28 g (82%) of desired bromide as yellow crystals, mp 219-221° C. 4-(8-Cyano-5-methyl-7-(3-nitrophenyl)-3-oxo-2,3-dihydro-7H-thia zolo[3,2-a]pyridin-6-yl)-1-(ethoxycarbonyl-methyl)pyridinium bromide.

Elemental analysis: Found: C 51.48; H 3.96; N 9.94; Calcd. for C₂₄H₂₁BrN₄O₅S: C 51.72; H 3.80; N 10.05.

Example 20b

N,N-[(2,6-Dimethyl-4-o-methoxyphenyl-1,4-dihydropyridine-3,5-diyl)-ethoxycarbonyl]bis N,N-dimethyloctylammonium diiodide.

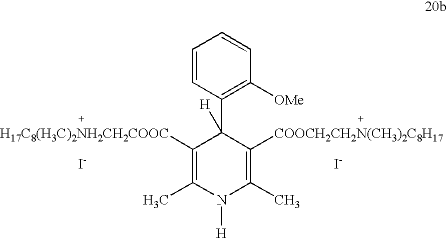

0.42 g (1 mmole) of 2,6-dimethyl-4-o-methoxyphenyl-3,5-di(2-chloro- ethoxycarbonyl)-1,4-dihydropyridine was dissolved with heating in 10 ml of methylethylketone and 0.42 ml (2 mmole) of N,N-di-methyloctylamine was added. Additional 0.33 g (2 mmole) powdered potassium iodide was added and the mixture was refluxed for 60 h. After cooling, the precipitate was filtered and recrystallized from the mixture 20:1 of acetone and methanol. 0.39 g (42%) of light yellow crystals was obtained. N,N′-{[1,4-Dihydro-4-(2-methoxyphenol)-2,6-dimethylpyridine-3,5-di-yl]bis-(carbonyloxyethylene)}-N,N,N′,N′-tetramethyl-N,N′-dioctyldiammonium diiodide. Melting point 220-223° C. C₄₀H₆₉I₂N₃O₅.

### EXAMPLE 21

2,6-Dibromomethyl-3,5-dialkoxycarbonyl(dicarbamoyl,dialkyl-thio)-4-aryl (heteryl)-1,4-dihydropyridines

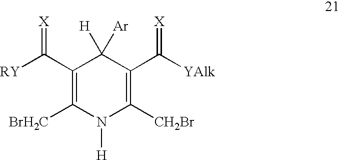

3,5-Didecyloxycarbonyl-4-phenyl-1,4-dihydropyridine decylace-to-acetate (2.42 g, 10 mmol), benzaldehyde (0.53 g, 5 mmol), 25% ammonium hydroxide solution (3.5 ml) in ethanol (25 ml) heated to refluxing 4 h and mixture was kept on cooling. The precipitate was filtered off and was obtained 1,4-dihydropyridine (2.0 g, 36%), m.p 55-57° C. The precipitate was used without further purification. Anal. Calcd. for C₃₅H₅₅NO₄. C 75.90, H 10.01, N 2.52. Found: C 75.40, H 9.80, 2.45.

Example 21a:

2,6-Dibromomethyl-3,5-didecyloxycarbonyl-4-phenyl-1,4-dihydropyridine

N-Bromosuccinimide (NBS) (0.5 g, 2.6 mmol) was added to solution of 2,6-dimethyl-3,5-didecyloxycarbonyl-4-phenyl-1,4-dihydropyridine (0.7 g, 1.3 mmol) in methanol (10 ml) at room temperature. The mixture was stirred at room temperature.The precipitate was filtered off and recrystallized in methanol, giving 2,6-dibromomethyl-1,4-dihydropyridine of Example 21a (0.4 g, 45%) m.p. 87-89° C. Didecyl-2,6-bis(bromomethyl)-1,4-dihydro-4-phenylpyridine-3,5-dicarboxylate. Anal.Calcd. for C₃₅H₅₃Br₂NO₄. C 59.17, H 7.50, N 1,96. Found: C 59.04, H 7.51, N 1.97.

Example 21b

2,6-Dibromomethyl-3,5-didodecyloxycarbonyl-4-phenyl-1,4-dihydropyridine

N-Bromosuccinimide (NBS) (0.6 g, 3.2 mmol) gradually was added to a solution of appropriate 2,6-dimethyl-1,4-dihydropyridine (1.0 g, 1.6 mmol) in methanol (100 ml) at 0° C. The mixture was stirred at 0° C. for 40 min, then mixture was diluted with water (40 ml) and kept at 4-6° C. The formed oil was separated and treated with hexane. The precipitate was filtered off and recovered was 0.44 g, 34% Didodecyl-2,6-bis(bromomethyl)-1,4-dihydro-4-phenylpyridine-3,5-dicarboxylate.

Example 21c:

2,6-Dibromomethyl-3,5-ditetradecyloxycarbonyl-4-phenyl-1,4-dihydropyridine 21c was prepared by bromination of appropriate 2,6-dimethyl-1,4-dihydropyridine with NBS following the procedure described for compound 2lb.

Ditetradecyl-2,6-bis(bromomethyl)-1,4-dihydro-4-phenylpyridine-3,5-dicarboxylate.

Yield: 38%.

Example 21d

2,6-Dibromomethyl-3,5-dihexadecyloxycarbonyl-4-phenyl-1,4-dihydropyridine 21d was prepared by bromination of appropriate 2,6-dimethyl-1,4-dihydropyridine with NBS following the procedure described for compound 21b.

Dihexadecyl-2,6-bis(bromomethyl)-1,4-dihydro-4-phenylpyridine-3,5-dicarboxylate.

Yield: 36%.

### EXAMPLE 22

1,1[(3,5-Dialkoxycarbonyl (dicarbamoyl,dialkylthiocarbonyl, dialkyldithiocarbonyl)-4-aryl(heteryl)-1-H(alkyl)-1,4-dihydropyridine-2,6-diyl)methylene]bispyridinium dihalides

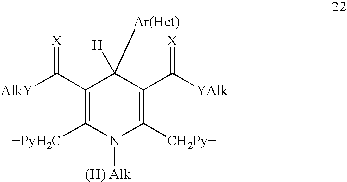

Example 22a (Derivative XXII):

1,1[(3,5-Didecyloxycarbonyl-4-phenyl-1,4-dihydropyridine-2,6-diyl)dimethylene]bispyridinium dibromide

Pyridine (0.2 ml, 1.2 mmol) was added to the solution of 2,6-dibromomethyl-3,5-didecyloxycarbonyl-4-phenyl-1,4-dihydropyridi ne (Example 21a) (0.4 g, 0.6 mmol) in dry acetone (10 ml). The mixture was stirred at room temperature for 3 h. The precipitate was obtained by cooling. It was recrystallized in methanol, giving bipyridinium dibromide (0.3 g, 63%), m.p. 156-158° C.

1,1′-{[3,5-Bis(decyloxycarbonyl)-1,4-dihydro-4-phenylpyridine-2,6-diyl]dimethylene}bispyridinium dibromide.

Anal. Calcd.for C₄₅H₆₃Br₂N₃O₄. C 62.13, H 7.30, N 4.83. Found: C 61.20, H 7.33, N 4.71.

Example 22b (Derivative XXIII):

1,1′-[(3,5-Didodecyloxycarbonyl-4-phenyl-1,4-dihydropyridine-2,6-diyl)dimethylene]bispyridinium dibromide

Pyridine (0.42 ml, 5.2 mmol) was added to a solution of 2,6-dibromomethyl-3,5-didodecyloxycarbonyl-4-phenyl-1,4-dihydropyridine prepared in Example 21 (0.2 g, 2.6 mmol) in acetone (15 ml). The mixture was stirred at room temperature for 4 h. The precipitate was filtered off and washed with acetone. The precipitate crystallized from ethanol and dried was then fractionally recrystallized from acetone. Bipyridinium dibromide prepared in example 22b (0.09 g, 40%) was obtained, m.p. 140-145° C.

1,1′-([3,5-Bis-(dodecyloxycarbonyl)-1,4-dihydro-4-phenylpyridine-2,6-diyl]dimethylene}bispyridinium dibromide.

¹H NMR (CDCl₃): δ 0.88(m,6H, OCH₂(CH₂)₁₀CH₃); 1.12-1.70(m,40H, OCH₂ (CH₂)₁₀CH₃); 4.07 (t, 4H, OCH₂(CH₂)₁₀CH₃); 5.08 (s, 1H, 4-H), 5.93 and 6.40 (AB-q,4H, J=11Hz, CH₂Py⁺Br—); 7.26(s,5H, Ph), 8.21(t,4H, S—H (Py)); 8.62(t,2H, α-H (Py)); 9.37(d,4H, α-H (Py)); 10.94(br.s, 1H, N—H).

Anal. Calcd.for C₄₉H₇₁Br₂N₃O₄. C 63.49, H 7.83, N 4.53. Found: C 63.24, H 7.76, N 4.45.

Example 22c (Derivative XXIV):

1,1′-[(3,5-Ditetradecyloxycarbonyl-4-phenyl-1,4-dihydropyridine-2,6-diyl)dimethylene]bispyridinium dibromide

Bipyridinium dibromide dihydrate 22c was prepared by reacting 21c with pyridine following the procedure for compound 22b. 1,1′-{[1,4-Dihydro-4-phenyl-3,5-bis(tetradecyloxycarbonyl)-pyridine-2,6-diyl]dimethylene}bispyridinium dibromide.

Yield: 35%), m.p. 144-147° C.

¹H NMR (CDCl₃): δ 0.88(m,6H, OCH₂(CH₂)₁₂CH₃); 1.11-1.66(m,48H, OCH₂(CH₂)₁₂CH₃); 4.02(t,4H, OCH₂(CH₂)₁₂CH₃); 5.06(s,1H, 4-H), 5.85 and 6.38 (AB-q,4H, J=11Hz, CH₂Py⁺Br—); 7.22(s,5H, Ph), 8.18(t,4H, —H (Py)); 8.59(t,2H, —H (Py)); 9.33(d,4H, a, α-H (Py)); 10.93(br.s,1H, N—H).

Anal. Calcd.for C₅₃H₇₉Br₂N₃O₄x2H₂O. C 62.53, H 8.22, N 4.13. Found: C 62.89, H 8.19, N 4.11.

Example 22d (Derivative XXV):

1,1[(3,5-Dihexadecyloxycarbonyl-4-phenyl-1,4-dihydropyridine-2,6-diyl)dimethylene]bispyridinium dibromide

Bipyridinium dibromide dihydrate 22d was prepared by reacting 21d with pyridine following the procedure for compound 22b.

Yield: 33%), m.p. 150-153° C.

¹H NMR (CDCl₃): δ 0.86(m,6H, OCH₂(CH₂)₁₄CH₃); 0.98-1.62(m,56H, OCH₂(CH₂)₁₄CH₃); 4.02(t,4H, OCH₂(CH₂)₁₄CH₃); 5.07(s,1H, 4-H), 5.82 and 6.31 (AB-q,4H, J=11Hz, CH₂Py⁺Br—); 7.18(s,5H, Ph), 8.17(t,4H, S—H (Py)); 8.58(t,2H, γ-H (Py)); 9.33(d,4H, α-H (Py)); 10.84(br.s,1H, N—H).

1,1′-{[3,5-Bis-(hexadecyloxycarbonyl)-1,4-dihydro-4-phenylpuridine-2,6-diyl]dimethylene}bispyridinium dibromide.

Anal. Calcd.for C₅₇H₈₇Br₂N₃O₄x2H₂O. C 63.73, H 8.54, N 3.91. Found: C 63.40, H 8.36, N 3.84.

B:

2,6-Dimethyl-3,5-dioctadec-9′-enyloxocarbonyl-4-phenyl-1,4-dihydropyridine

A mixture of octadec-β-enylacetoacetate (2.00 g, 5.67 mmol), benzaldehyde (0.30 g, 2.84 mmol) and ammonia (1.55 ml, 22.90 mmol, 28% solution in water) in MeOH (10 ml) was refluxed under argon 3 h and evaporated to dryness in vacuo. The residue was purified by TLC on silica gel (Acros, 0.035-0.070 mm, 6 A) plate (1.5 150 300 mm). Eluent—hexane/EtOAc (4:1). Light brown oil was obtained. Yield 1.60 g (73%).

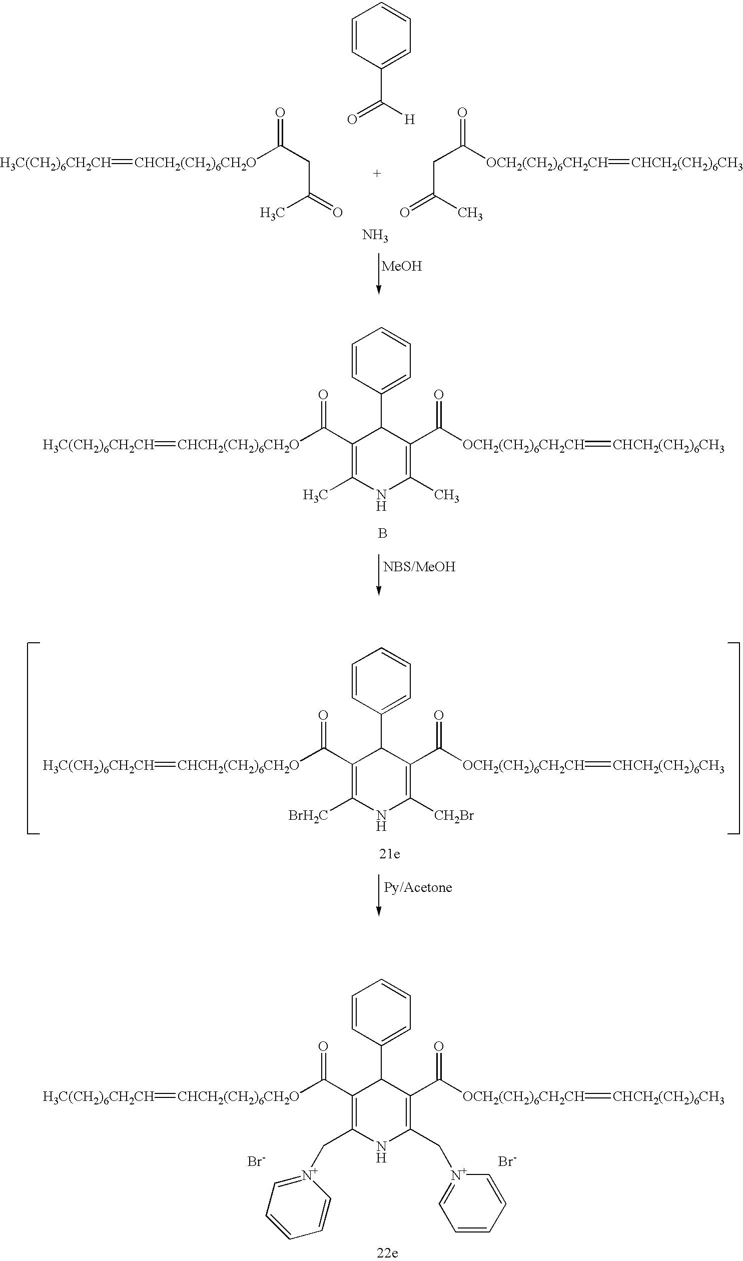

Example 21e

2,6-Dibromomethyl-3,5-dioctadec-9′-enyloxocarbonyl-4-phenyl-1,4-dihydropyridine

and

Example 22e (Derivative XXVI)

1,1′-[(3,5-Dioctadec-9′-enyloxycarbonyl-4-phenyl-1,4-dihydropyridine-2,6-diyl)dimethylene]bispyridinium dibromide N-bromosuccinimide (NBS) (0.209, 1.12 mmol) was added to a solution of 2,6-dimethyl-1,4-dihydropyridine (B) (0.40 g, 0.52 mmol) in MeOH (400 ml). The mixture was stirred at r.t. for 2 h, then the mixture was diluted with water (100 ml) and evaporated MeOH in vacuo. The residue was light brown oil 21e 0.25 g. The oil was dissolved in acetone (10 ml) and pyridine (0.040 g, 0.51 mmol) was added. Mixture was stirred at r.t. for 3.5 h. The mixture was purified by TLC on silica gel (Acros, 0.035-0.070 mm, 6 Å, was treated with saturated NaBr solution in MeOH for 5 min) plate (1.5 150 300 mm). Eluent—CH₂Cl₂/MeCN (2:1). Light brown oil was obtained. Yield 0.060 g (10.66%).

1,1′-{[4-Dihydro-3,5-bis(octadec-β-enyloxycarbonyl)-4-phenylpyridine-2,6-diyl]dimethylene}bispyridinium dibromide.

Example 22f:

1,1′-[(4-(2-Difluoromethoxyphenyl)-3,5-dimethoxycarbonyl-1-methyl-1,4-dihydropyridine-2,6-diyl)dimethylene]bis-pyridinium dibromide

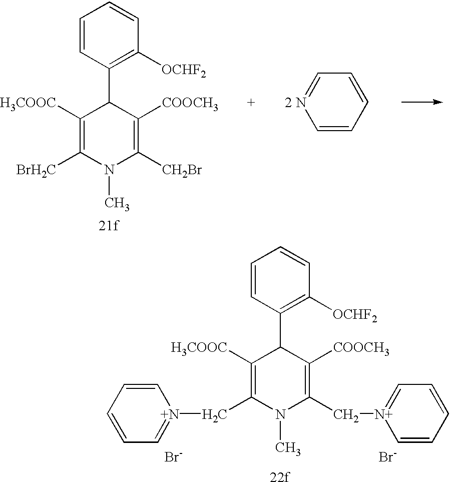

Derivative of Example 21f (4.04 g, 7.5 mmol) was dissolved in 75 ml of acetonitrile, pyridine (1.21 ml, 15.0 mmol) was added, and the mixture was stirred at ambient temperature for 1 hour, then left overnight. The precipitate was filtered off and recrystallized from MeOH/Et₂O to give colourless crystals of 22f (3.36 g, yield 64%). M.p. 195-210° C. (decomp.).

1,1′-{{4-[2-(Difluoromethoxy)phenyl]-1,4-dihydro-3,5-bis(methoxycarbonyl)-1-methylpyridine-2,6-diyl}dimethylenelbispyridinium dibromide.

Anal. Calcd.for C₂₉H₂₉Br₂F₂N₃O₅. C 49.95, H 4.19, N 6.03. Found: C 48.59, H 4.27, N 5.83.

Example 22g:

1,1′-{{4-[2-(Difluoromethoxy)phenyl]-1,4-dihydro-3,5-bis(methoxycarbonyl)-pyridine-2,6-diyl}dimethylene}bispyridinium dibromide was prepared correspondingly.

Example 22h:

1,1′-t[3,5-Bis(dodecyloxycarbonyl)-1,4-dihydro-1-methyl-4-phenylpyridine-2,6-diyl]dimethylenelbispyridinium dibromide was prepared correspondingly.

Example 22i:

1,1′-{[3,5-Bis(ethoxycarbonyl)-1,4-dihydro-4-phenylpyridine-2,6-diyl]dimethylene}bispyrazinium dibromide was prepared correspondingly.

### EXAMPLE 23

N,N′-[(3,5-Dialkoxycarbonyl(dicarbamoyl,dialkylthiocarbonyl)-1-H-(alkyl)-4-aryl(heteryl)-1,4-dihydropyridine-2,6-diyl)-dimethylene]-bistrialkylammonium dibromides

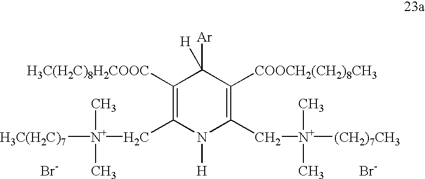

Example 23a (Derivative XXVII):

N,N′-[(3,5-Didecyloxycarbonyl-4-phenyl-1,4-dihydropyridine-2,6-diyl)dimethylene]bis-N,N-dimethyloctylammonium dibromides 0.88 g (5.6 mmole) N,N-dimethyloctylamine was added by stirring to a solution of 1 g (1.4 mmole) of 2,6-dibromomethyl-3,5-didecyloxy-carbonyl-4-phenyl-1,4-dihydropyridine in 5 ml of anhydrous acetone. The mixture was stirred at room temperature for 24 h, and after cooling the precipitate was filtered and washed with dry acetone giving N,N-[(3,5-didecyloxycarbony-1-4-phenyl-1,4-dihydro- pyridin-2,6-diyl)dimethyl]bisdimethyloctylammonium dibromide of Example 23a 0.4 g (32% yield), m.p. 138-140° C. (from acetone/anhydrous ethanol).

N,N′-{[3,5-Bis(decyloxycarbonyl)-1,4-dihydro-4-phenyl-pyridine-2,6-diyl]dimethylene}-N,N,N′,N′-tetramethyl-N,N′-dioctyldiammonium dibromide.

Anal. Calcd. For C₅₅H₉₉Br₂N₃O₄: C 64.37; H 9.72; N 4.09; Br 15.57. Found: C 65.10; H9.90; N 3.71.

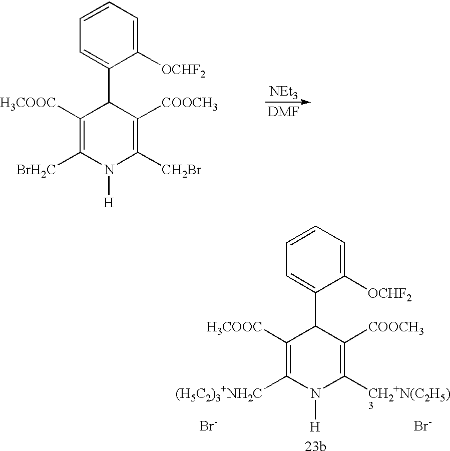

Example 23b

N,N-[(4-(2-Difluoromethoxyphenyl)-3,5-dimethoxycarbonyl-1,4-dihydropyridine-2,6-diyl) dimethylene]bistriethylammonium dibromides

To the solution of 2,6-dibromomethyl-4-(2-difluoromethoxyphenyl)-3,5-dimethoxycarbonyl-1,4-dihydropyridine (1.0 g, 1.9 mmol) in dimethyl formamide (10 ml) the triethylamine (1.1 ml, 7.6 mmol) was added. After stirring of the reaction mixture for 3 h at room temperature the orange precipitate formed was filtered off and recrystallized from chloroformhexane to afford 1.13 g (82%) of salt 23b, m.p. 191-194° C. N,N′-{{4-[2-(Difluoromethoxy)phenyl]-1,4-dihydro-3,5-bis(methoxycarbonyl)-pyridine-2,6-diyl}dimethylene}-N,N,N,N′,N′,N′-hexaethyldiammonium dibromide.

Anal. Calcd. For C₃₀H₄₇Br₂F₂N₃O₅: C 49.54; H 6.46; N 5.78; Found: C 49.01; H 6.19; N 5.54.

Example 23c:

N,N′-{[3,5-Bis(ethoxycarbonyl)-1,4-dihydro-3,5-bis(methoxycarbonyl)-pyridine-2,6-diyl}dimethylene}-N,N,N,N′,N′,N′-hexaethyldiammonium dibromide

### EXAMPLE 24

1-Hexadecyl-3-(1′-adamanthyloxycarbonyl)-1,4-dihydrobenzothieno-[3,2-b]-pyridyl-5,5 dioxide-4}-pyridinium bromide

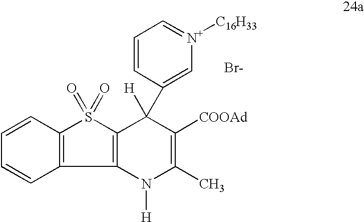

Example 24a:

3 mmoles (1.54 g) of 4-(3-pyridyl)-1,4-dihydrobenzthieno[3,2-b]-pyridine-5,5-dioxide was dissolved with heating in 50 ml methylethylketone and 3 mmoles n-hexadecyl bromide (0.9 ml, 0.9 g) was added. The mixture was refluxed for 50 h. After cooling the yellow precipitate was filtered. Recrystallization from methylethylketone gave 1.0 g of target compound (40.8%) m.p. 177-178° C.

3-[3- (1-Adamantyloxycarbonyl)-2-methyl-5,5-dioxo-4,5-dihydro-1H -benzo[4,5]thieno[3,2-b]pyridin-4-yl]-1-hexadecylpyridinium bromide.

### EXAMPLE 25

Preparation of Liposomes

Cationic liposomes comprising derivatives I-XXI were prepared by dissolving the crystalline derivative in chloroform. The solvent was evaporated under a stream of nitrogen for 45 min in vacuum. The resulting thin films were resuspended in 4 ml deionized water, vortexed and sonicated in bath type sonicator 30′ until the solution became clear. (The final concentration of liposomes was 2.5 mM.) In order to prepare the liposomes of derivatives I-XXI (Table 2) high temperatures (40-65° C.) were used. Derivatives XXII-XXVII (Table 2) were dissolved in deionized water, vortexed and sonicated 5-7 min in a bath sonicator.

Most of the tested derivatives showed self-association properties and formed liposomes -n a aqueous media as shown by light scattering measurements. The mean sizes of the self-associating structure were 35-2000 nm.

DOPE and PEG containing XXIII liposome were prepared by dissolving all the components in chloroform and subsequently the solvent was evaporated under a nitrogen stream to form a thin lipid film. MES-Hepes buffer or 5% glucose was added and the lipid solutions were vortexed and sonicated to clarity.

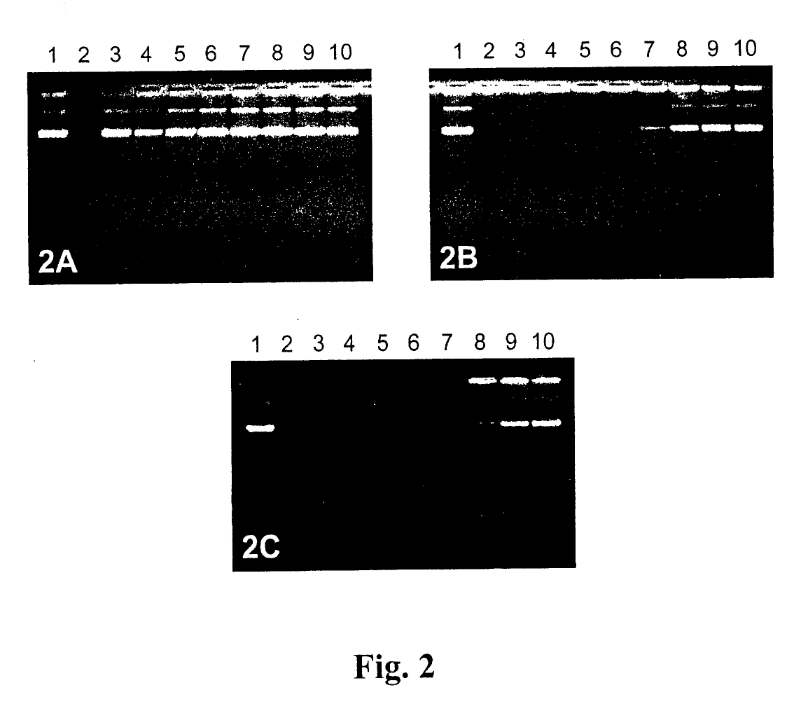

Sizes of the freshly prepared amphiphile/plasmid DNA complexes in MES-HEPES buffer (pH 7.2), water and 5% (w/v) glucose were determined and compared with DNA complexes of PEI 25 and DOT-AP. The results show that in MES-HEPES buffer, the mean diameters of the complexes at high (≧4) +/−ratios are rather small (<150 nm) for most of the compounds (FIG. 2A). The sizes remarkably in- crease up to 10-35-fold with decreasing +/−ratio. The mean sizes are at maximum at charge ratios 2:1 and 1:1 (+/−) and in most cases decrease again at negative charge excess (+/−0.5). At low +/−ratios, the complexes of all examined amphiphiles, DOTAP and PEI 25 were stable in buffer only for a short time: already after 3-4 h, or at latest 24 h, visible aggregates were formed. This was not observed at high (≧8) charge ratios. Since DNA is more complexed at the charge close to neutrality, the repulsion between liposomes is reduced. This is leading to fusion and aggregation and results in enhancement of sizes of complexes. The complexes prepared in water (FIG. 2B) or in 5% (w/v) glucose (FIG. 2C) were smaller (15-120 nm) and their sizes remained at about the same level for 10 days. The sizes were small even at the charges close to neutrality or at the excess of DNA. Similar observations were found for DOTAP/DNA and PEI 25/DNA complexes (FIG. 2A FIG. 2C), although PEI 25 complexes were too small (<15 nm) in water and in 5% (w/v) glucose solution for accurate determination with the light scattering method.

### EXAMPLE 26

Preparation of Liposome/Nucleic Acid Complexes for in vitro Transfection

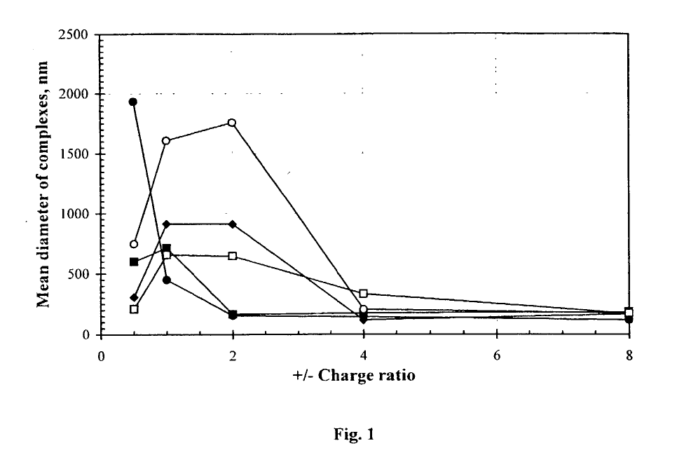

The cationic liposome/plasmid DNA with or without PEG or DOPE complexes were prepared by adding of 0.6 μg of DNA to different concentrations of liposomes to obtain different +/−charge ratios of 0.5-16. The complexes were made in MES-HEPES, pH 7.2 and the size distribution of complexes are shown in FIG. 1. FIG. 1 shows the influence of +/−charge ratio on the size of complexes of DNA with cationic amphiphiles composed of: comp. I: DOPE (▪) comp. VI (□), comp. VI DOPE (●), comp. XXIII (♦) and DOTAP (◯) in MES-HEPES, pH 7.2.

### EXAMPLE 27

Preparation of Plasmid/Carrier Complexes for in vivo Transfection.

For in vivo studies derivative/plasmid DNA complexes were prepared by the above mentioned method. After gentle swirling the mixtures were allowed to stand in room temperatures for 30 min. prior to use. The complexes were made in phosphate buffer saline and those having +/−charge ratios of 2-8 were used.

### EXAMPLE 28

Complexation of DNA

Complexation of DNA was demonstrated by using gel mobility assay and DNA condensation test. The 1,4-dihydropyridine derivative/DNA complexes were prepared at different charge ratios. After 25 min, a buffer with bromphenol blue was added and the complexes were loaded on 0.9% agarose gel in Tris-borate EDTA buffer (TBE), pH 8.0. Voltage of 65 V was applied for 3 h and after EtBr staining DNA bands were visualized.

The derivatives were able to complex DNA as shown in FIG. 2 depicting the electrophoresis. During gel electrophoresis complexed DNA did not migrate in the electric field like free plasmid DNA.

The effect of pegylation was studied with 0, 0.5, 1 and 2 mol % DOPE-PEG. The results indicated that pegylation did not interfere with the plasmid complexation. Except for DOPE containing complexes slight migration for plasmid DNA was observed at charge ratios +/−4 and 2. FIG. 2 depicts a gel electrophoresis of 1,4-dihydropyridine derivatives/DNA complexes: compound V (panel A), compound V: DOPE (panel B), compound XXIII (panel C). In each panel: (line A) pCVMβ plasmid DNA (0.6 μg) alone (positive control), (line 2) compound (75 μM) alone (negative control), (lines 3-10) compound/DNA complexes at charge ratios +/−: 16; 8; 4; 2; 1; 0.3; 0.25; 0.125 respectively.

### EXAMPLE 29

DNA Condensation

All examined derivatives are characterized by their ability condense DNA. The ability of the cationic amphiphiles to condense DNA was assessed with an EtBr displacement assay. Briefly, in 96 well plates plasmid DNA (0.6 μg per well) was reacted with 0.002% EtBr in 20 mM HEPES—150 mM NaCl buffer, pH 7.4 and fluorescence intensity was measured at 530 nm (excitation) and 590 nm (emission). Intercalation of EtBr molecules between the base pairs of the DNA double helix results in increased fluorescence signal. Immediately thereafter, the cationic liposomes were added to form complexes at different +/−charge ratios and the quenching of fluorescence intensity of EtBr was monitored. Condensation of DNA upon complexation with liposomes results in the displacement of EtBr from DNA and in decreased fluorescence signal. Fluorescence was measured using FL 500 microplate fluorescence reader (Bio-Tek Instruments Inc., Winooski, Vt., USA).

The results shown in FIG. 3 indicate that double-charged derivatives XXII-XXVII) condense DNA more efficiently than single-charged derivatives I-XXI.

FIG. 3 shows the DNA condensation ability of cationic amphiphiles. (A) compound XXII (♦), compound XXIII (●), compound XXIV (▪), compound XXV (◯) DOTAP (x), Lipofectin (□) (B) compound V (♦), compound VI (●), compound VII (◯), compound I (▪) (C) compound V: DOPE (♦), compound VI: DOPE (●), compound VII: DOPE (◯), compound I: DOPE (▪).

The values are expressed as percentage of the maximum fluorescence signal when EtBr bound to DNA in the absence of an amphiphile. Each data point is from at least triplicate experiment ±S.D.

### EXAMPLE 30

DNA Transfer to the Cells in vitro

Transfer of plasmid DNA by various compounds was investigated by using CV-1P (African monkey green kidney fibroblasts cells) and D407 (human retinal pigment epithelial) cells.

The cells were cultured in Dulbecco's modified Eagle's medium (DMEM, Gibco), supplemented with 10% fetal bovine serum (FBS), penicillin/streptomycin (100 units/ml and 100 μg/ml respectively) and 2 mM L-glutamine for D 407 cell line. Cell cultures were maintained at 37° C. in a 7% CO₂/air incubator. The cells were collected, counted and seeded in growth medium (100 μl) into 96-well culture plate, 20 000 cells per well. One day later the medium was replaced with fresh medium without serum (150 μl)

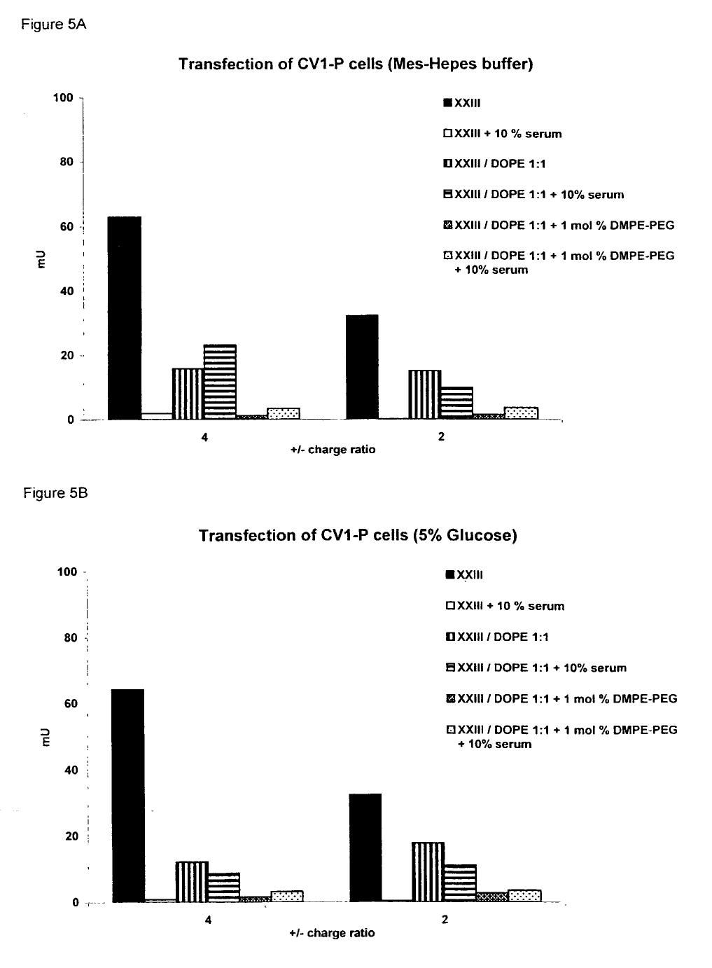

Transfection of CV-1P cells in vitro shows that DOPE containing complexes of compound XXIII can transfect the cells efficiently both in the serum conditions and in the presence of 10% serum (FIG. 5). Also, DMPE-PEG containing liposomes could retain some transfection activity, which was also serum independent (FIG. 5).

The complexes can be prepared either in buffer (e.g. MES-HEPES) or in isotonic glucose solution. The resulting complexes made in isotonic glucose solution were smaller (below 80 nm in diameter) than the complexes prepared in MES-HEPES buffer (100-500 nm) The small complex size may provide better distribution into the tissues, but it did not interfere with gene transfer efficacy into the cells (FIG. 5A and FIG. 5B) which show the effect of serum, DOPE and pegylated lipid on transfection into subconfluent CV1-P cells. The complexes were prepared in Mes-Hepes buffer (FIG. 5A) or 5% Glucose (FIG. 5B) in CV1-P cell line. Transfection levels are given as 9-galactosidase units (mU) per well.

Confluent cells were transfected similarly to the subconfluent cells. Without serum the highest level of beta-galactosidase expression (208 mU) was seen with compound 23 without DOPE. addition of DOPE decreased the gene expression to 10-20 mU levels, but this level was obtained also in the presence of 10%.

In the case of confluent CV1-P cells the medium (with of without serum) was changed on fourth day of cell growth before the transfection.

The complexes at different derivative/DNA charge ratios for transfection procedure were prepared just before use in 50 mM MES—50 mM HEPES—75 mM NaCl buffer, pH 7.2 in separate 96 well plates. One hour after changing the serum-free medium with 10% serum the complexes were added to the cells at 370C. Dose of DNA per well was 0.6 μg. After 5 h the complexes were removed, the cells were washed with PBS, and the normal growth medium was added. After 45 h of incubation at 37° C. the cells were lysed with 2% Triton X 100, twice deep frozen and the β-galactosidase activity in each well was determined spectrofotometrically with ELx800 automated microplate reader (Bio-Tex Instruments Inc., Winooski, Vt. USA) by monitoring the hydrolysis of o-nitrophenylgalactopyranoside (oNPG) at 405 nm. Purified β-galactosidase from E.coli was used to construct a standard curve and to calculate 9-galactosidase activity in the transfected cells. The results are summarized in Table 3 and FIG. 4 in which the efficiencies of transfection of CVI-P cells (FIG. 4A) and D407 cells (FIG. 4B) using compound XXIII and DOTAP are shown. Transfection efficiencies are given as percentage in comparison to transfection efficiency of Lipofectin^{R}.

In vitro gene transfection results showed that transfection efficiencies were dependent on the cell line. With both examined cell lines the double charged amphiphiles were more effective than single charged (Table 3). The level of transfection of amphiphile XXIII obtained with both cell lines were at least twenty times higher than that of Lipofectin^{R} and with CV1-p cells ten times higher than transfection efficiency with DOTAP (FIG. 4).

### EXAMPLE 31

In vivo Transfection Studies

The complexes were prepared using the method described above.

Five New Zealand White male rabbits of 2.5-3.5 kg were used. Fentanyl-fluanisone (0,3 mL/kg, s.c., Janssen Pharmaceutica, Beerse, Belgium) and midazolam (1.5 mg/kg, i.m., Roche, Basel, Switzerland) were used for anesthesia. Carotid arteries were exposed using midline neck incision. Arteries were carefully separated from the surrounding tissue and 3 cm silastic collar (MediGene Oy, Kuopio, Finland) was positioned around the artery. Rabbits were re-anesthetized for gene transfer, which was performed 5 days after the installation of the collars. The collars were opened and filled with 600 μL of the gene transfer solution. Rabbits were sacrificed three days after the gene transfer and arteries were removed for histological analyses. All animal procedures were approved by Animal Care and Use Committee, University of Kuopio, Finland.

Histological Analysis

Collared arteries were divided into three equal parts: the proximal third was immersion-fixed in 4% paraformaldehyde/15% sucrose (pH 7.4) for 4 h, rinsed in 15% sucrose (pH 7.4) overnight and embedded in paraffin. The medial third was stored at −70° C. for later analyses. The distal third was immersion-fixed in 4% paraformaldehyde/phosphate buffered saline (pH 7.4) for 30 min, rinsed 24 h in phosphate buffer (pH 7.2) and embedded in OCT compound.

Ten randomly selected frozen sections (10 μm) from each rabbit were stained with X-gal for 18 h to identify β-galactosidase positive cells. Mayers Carmalum-stain was, used as counter stain. The images were taken with Olympus AX70 microscope (Olympus Optical, Tokyo, Japan) using Image-Pro Plus™ software (Media Cybernetics, Silver Spring, U.S.A.). Animals received derivative XXIII/plasmid complexes prepared at three different charge ratios. β-galactosidase activity was detected at all study groups. Positive cells were mostly located at adventitia, some animals showed β-gal expression also in media. Transfected cells are probably fibroblasts and smooth muscle cells. Charge ratio +4 was found to be most effective. Gene transfer efficiencies were between 0.05-1.5%

In vivo gene transfer using derivative XXIII/plasmid complexes results in marker gene expression in the target arteries. We have previously used DOTMA/DOPE (Lipofectim™) liposomes in this animal model and we found that they result in 0.05% gene transfer efficiency.

TABLE 3

| | Amphiphile/DNA +/− charge ratio | | | | | | |
|---|---|---|---|---|---|---|---|
| Compound | 8 | 4 | 2 | 1 | 0.5 | Cell line | Activity |
| I | 0,78 | 0,73 | 2,89 | 7,72 | 0,26 | CVI-P | transfection¹ |
| | 0,88 | 0,89 | 5,72 | 20,04 | 0,83 | D 407 | cytotoxicity² |
| | 70,75 | 78,43 | 91,92 | 94,83 | 94,43 | CVI-P | complexation³ |
| | 26,92 | 31,39 | 51,14 | 80,92 | 90,43 | D 407 | |
| | 73,35 | 78,3 | 84,4 | 87,2 | 89,4 | | |
| I:DOPE | 0,91 | 2,32 | 8,07 | 11,94 | 5,48 | CVI-P | transfection |
| (1:1) | 1,36 | 0,32 | 1,22 | 3,17 | 1,15 | D 407 | cytotoxicity |
| | 27,0 | 69,22 | 83,54 | 85,17 | 91,9 | CVI-P | complexation |
| | 14,71 | 13,89 | 36,11 | 61,83 | 84,52 | D 407 | |
| | 78,95 | 81,35 | 89,35 | 94,75 | 99,45 | | |
| II | 99,23 | 79,08 | No signal | 101,24 | 111,42 | CVI-P | transfection |
| | 72,4 | 74,8 | 102,9 | 85,6 | 92,2 | CVI-P | cytotoxicity |
| | | | 77,2 | | | | complexation |
| III | 68,47 | 54,84 | No signal | 81,86 | 87,86 | CVI-P | transfection |
| | 74,7 | 74,6 | 72,04 | 83,2 | 93,2 | CVI-P | cytotoxicity |
| | | | 76,7 | | | | complexation |
| IV | | Fast | No signal | crystalls | | CVI-P | transfection |
| | | | residue & | | | | |
| V | 1,4 | 5,95 | 27,19 | 0,69 | 0,48 | CVI-P | transfection |
| | 0,33 | 0,55 | 8,78 | 8,10 | 0,26 | D 407 | cytotoxicity |
| | 8,67 | 40,55 | 71,79 | 87,18 | 100,21 | CVI-P | complexation |
| | 0,28 | 9,14 | 55,61 | 66,05 | 85,27 | D 407 | |
| | 72,4 | 71,93 | 75,17 | 80,33 | 88,8 | | |
| V:DOPE | 0,41 | 0,84 | 0,65 | 0,81 | 0,21 | CVI-P | transfection |
| (1:1) | 0,36 | 0,26 | 1,03 | 5,84 | 0,35 | D 407 | cytotoxicity |
| | 4,8 | 34,8 | 69,95 | 76,35 | 99,95 | CVI-P | complexation |
| | 1,39 | 8,23 | 28,97 | 63,61 | 90,2 | D 407 | |
| | 86,07 | 83,07 | 88,03 | 94,8 | 95,77 | | |
| VI | 1,27 | 2,16 | 4,82 | 0,82 | 0,81 | CVI-P | transfection |
| | 1,08 | 1,21 | 35,6 | 16,56 | 0,53 | D 407 | cytotoxicity |
| | 7,52 | 45,6 | 90,38 | 89,46 | 94,05 | CVI-P | complexation |
| | 5,11 | 12,13 | 79,12 | 94,43 | 98,6 | D 407 | |
| | 71,05 | 74,85 | 78,9 | 84,75 | 93,25 | | |
| VI:DOPE | 0,29 | 2,17 | 1,5 | 1,9 | 0,02 | CVI-P | transfection |
| (1:1) | 0,40 | 0,20 | 3,34 | 12,78 | 1,95 | D 407 | cytotoxicity |
| | 4,78 | 75,59 | 77,93 | 85,65 | 93,07 | CVI-P | complexation |
| | 3,41 | 15,06 | 36,92 | 68,47 | 89,56 | D 407 | |
| | 77,45 | 76,5 | 83,0 | 89,05 | 92,67 | | |
| VII | 0,61 | 0,81 | 2,2 | 0,39 | 0,73 | CVI-P | transfection |
| | 0,63 | 0,98 | 1,37 | 0,82 | 0,4 1 | D 407 | cytotoxicity |
| | 88,71 | 99,84 | 103,62 | 97,03 | 102,34 | CVI-P | complexation |
| | 51,38 | 55,53 | 62,61 | 84,83 | 95,82 | D 407 | |
| | 77,6 | 79,25 | 83,11 | 86,95 | 90,6 | | |
| VII:DOPE | 0,41 | 1,78 | 8,06 | 8,35 | 0,26 | CVI-P | transfection |
| (1:1) | 0,44 | 0,27 | 1,91 | 9,72 | 0,47 | D 407 | cytotoxicity |
| | 7,65 | 70,89 | 86,22 | 90,6 | 96,7 | CVI-P | complexation |
| | 1,80 | 13,31 | 36,39 | 64,15 | 95,39 | D 407 | |
| | 83,1 | 82,8 | 89,0 | 93,1 | 92,1 | | |
| VIII | | | do not | complexes | DNA | | transfection |
| | | | | | | | cytotoxicity |
| | | | | | | | complexation |
| IX | 100,65 | 106,62 | No signal | 84,63 | 83,11 | CVI-P | transfection |
| | 98,5 | 97,8 | 97,34 | 101,6 | 101,6 | CVI-P | cytotoxicity |
| | | | 103,3 | | | | complexation |
| | 124,54 | 117,42 | No signal | 107,13 | 90,48 | CVI-P | transfection |
| | 106,1 | 98,4 | 120,34 | 97,5 | 97,8 | CVI-P | cytotoxicity |
| | | | 97,5 | | | | complexation |
| X | | Toxic, | No signal | cell viable | | CVI-P | transfection |
| | | | at +/− | | | CVI-P | cytotoxicity |
| | | | 50% | | | | |
| XI | | Toxic, | No signal | cell viable | | CVI-P | transfection |
| | | | at +/− | | | CVI-P | cytotoxicity |
| | | | 50% | | | | |
| XII | | No | solution | can be | pre- | | |
| | | vesicle | | | pared | | |
| XIII | | | No signal | complexes | DNA | CVI-P | transfection |
| | | | do not | | | | complexation |
| XIV | — | — | 1,5 | 3,38 | 0,75 | CVI-P | transfection |
| | 44,62 | 34,5 | 51,04 | 93,16 | 103,98 | CVI-P | cytotoxicity |
| | 93,7 | 88,9 | 91,03 | 96,2 | 100 | | complexation |
| XV | | | No signal | complexes | DNA | CVI-P | transfection |
| | | | do not | | | | complexation |
| XVI | | Toxic, | No signal | cell viable | DNA | CVI-P | transfection |
| | | | at +/− | complexes | | CVI-P | cytotoxicity |
| | | | 50% | | | | complexation |
| | | | do not | | | | |
| XVII | 97,34 | 67,45 | No signal | 85,31 | 105,14 | CVI-P | transfection |
| | 96,5 | 97 | 88,20 | 97,9 | 104,4 | CVI-P | cytotoxicity |
| | | | 95,1 | | | | complexation |
| XVII:DOPE | — | 3,2 | 2,9 | — | — | CVI-P | transfection |
| (1:1) | 89,73 | 53,46 | 57,45 | 72,98 | 96,55 | CVI-P | cytotoxicity |
| | 105,3 | 96,0 | 93,8 | 98,2 | 103,1 | | complexation |
| XVIII | 1,1 | 0,94 | 2,36 | 0,52 | 0,54 | CVI-P | transfection |
| | 3,05 | 9,08 | 76,51 | 83,94 | 85,15 | CVI-P | cytotoxicity |
| | 79,6 | 78,5 | 82,6 | 90,8 | 97,5 | | complexation |
| XVIII:DOPE | 0,59 | 0,96 | 7,24 | 4,06 | 2,14 | CVI-P | transfection |
| (1:1) | 2,86 | 9,21 | 29,04 | 77,9 | 83,3 | CVI-P | cytotoxicity |
| | 107,4 | 101,4 | 93,8 | 94,5 | 98,6 | | complexation |
| XIX | | | do not | complexes | DNA | | complexation |
| XX | 92,09 | 71,98 | No signal | 110,76 | 100,59 | CVI-P | transfection |
| | 66,31 | 71,5 | 59,96 | 81,94 | 88,0 | CVI-P | cytotoxicity |
| | | | 76,4 | | | | complexation |
| XXI | | | No signal | | | CVI-P | transfection |
| XXII | 46,74 | 78,2 | No signal | 87,2 | 95,71 | CVI-P | transfection |
| | 43,5 | 45,1 | 84,01 | 58,8 | 77,8 | CVI-P | cytotoxicity |
| | | | 48,1 | | | | complexation |
| XXIII | 22,33 | 122,8 | 65,5 | 0,42 | 0,44 | CVI-P | transfection |
| | 4,43 | 35,82 | 0,6 | 0,27 | 0,27 | D 407 | cytotoxicity |
| | 42,72 | 89,35 | 93,48 | 94,54 | 99,71 | CVI-P | complexation |
| | 11,98 | 70,23 | 79,68 | 87,69 | 93,67 | D 407 | |
| | 42,48 | 44,14 | 48,3 | 59,03 | 78,22 | | |
| XXIV | 9,48 | 47,78 | 15,46 | 2,48 | 0,63 | CVI-P | transfection |
| | 3,61 | 15,04 | 0,53 | 0,44 | 0,20 | D 407 | cytotoxicity |
| | 48,08 | 88,19 | 86,72 | 98,12 | 96,17 | CVI-P | complexation |
| | 51,24 | 86,99 | 98,83 | 88,59 | 97,59 | D 407 | |
| | 49,62 | 50,0 | 54,5 | 63,92 | 78,91 | | |
| XXV | 9,04 | 15,15 | 17,5 | 0,24 | 0,23 | CVI-P | transfection |
| | 2,47 | 2,05 | 066 | 0,31 | 0,57 | D 407 | cytotoxicity |
| | 100,65 | 107,03 | 101,03 | 99,29 | 92,82 | CVI-P | complexation |
| | 62,05 | 89,12 | 101,48 | 97,73 | 97,52 | D 407 | |
| | 71,37 | 77,14 | 83,1 | 89,06 | 94,16 | | |
| XXVI | 91,78 | 99,85 | No signal | 102,89 | 105,06 | CVI-P | transfection |
| | | | 107,28 | complexes | DNA | CVI-P | cytotoxicity |
| | | | do not | | | | complexation |
| XXVII | 3,14 | 21,46 | 36,55 | 1,63 | 0,24 | CVI-P | transfection |
| | 13,46 | 24,62 | 88,41 | 98,37 | 105,03 | CVI-P | cytotoxicity |
| | | | | | | | complexation |
| DOTAP | 3,19 | 10,5 | 15,58 | 0,9 | 0,61 | CVI-P | transfection |
| | 4,92 | 19,6 | 25,05 | 4,8 | 0,7 | D 407 | cytotoxicity |
| | 102,3 | 97,89 | 91,87 | 92,09 | 92,45 | CVI-P | complexation |
| | 76,11 | 81,34 | 80,32 | 87,48 | 85,08 | D 407 | |
| | 65,47 | 65,5 6 | 69,15 | 76,11 | 86,72 | | |
| Lipofectin | 2,32 | 5,40 | 4,78 | 1,19 | 0,83 | CVI-P | transfection |
| | — | 1,83 | 1,86 | — | — | D 407 | cytotoxicity |
| | 54,27 | 95,44 | 92,07 | 99,71 | 99,51 | CVI-P | complexation |
| | 95,1 | 95,44 | 97,2 | 99,71 | 99,51 | D 407 | |
| | 79,47 | 81,1 | 86,96 | 91,99 | 96,68 | | |

¹transfection efficiencies are given as β-galactosidase units per well
²cytotoxicity as a percentage of survived cells
³complexation values are expressed as percentage of the maximum fluorescence signal when EtBr bound to DNA in the absence of an amphiphile

1. A composition for delivering nucleotide containing compounds into a target cell and/or its nucleus, characterized in that it comprises a nucleotide containing compound complexed with one or more cationic, amphiphilic 1,4-dihydropyridine derivative, said 1,4-dihydropyridine derivative having the general formula I, 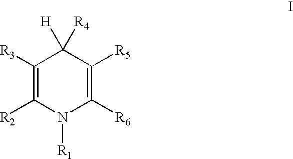
wherein
R₁ is hydrogen, (C₁-C₁₆), preferably (C₈-C₁₄), most preferably (C₁₀-C₁₂)alkyl, aralkyl or aryl, selected from a group consisting of phenyl, substituted phenyl, naphthyl, acylCO(C₁-C₁₆), preferably (C₈-C₁₄), most preferably (C₁₀-C₁₂)alkyl and COaryl;
R₂ is (C₁-C₁₀), preferably (C₁-C₃)alkyl or CH₂X;
wherein
X is pyridinio (C₅H₅N⁺), substituted pyridinio, diazinio (C₄H₄N₂⁺), substituted diazinio, trialkyl(C₁-C₁₀), preferably (C₁-C₃)ammonio, a (C₁-C₁₆), preferably (C₈-C₁₄)1 most preferably (C₁₀-C₁₂)alkylthio group or an alkylthio group with a carbonyl function, selected from a group consisting of S(CH₂)ₙCONH₂, S(CH₂)ₙCOAr and S(CH₂)ₙCOO(C₁-C₁₆), preferably (C₈-CO₄), most preferably (C₁₀-C₁₂)alkyl;
wherein
n is an integer from 1 to 16, preferably 8-14, most preferably 10-12;
R₃ is a cyano or nitril group or C(═Y)—(Z)ₙR₇ with a carbonyl function;
wherein
Y is O or S;
Z is O, S or NH or NR₇;
n is an integer 0 or 1; and
R₇ is saturated or unsaturated (C₁-C₁₀), preferably (C₈-C₁₄), most preferably (C₁₀-C₁₂)alkyl, a derivative of cyclohexane, a terpene selected from a group consisting of bornyl, i-bornyl, menthyl, steryl, cholesteryl, adamantyl, aralkyl(C₁-C₃)alkylAr;
wherein
Ar means aryl, alkoxyalkyl(C₁-C₆), preferably (C₁-C₃)alkyl-O—(C₁-C₃)alkyl, alkanoyloxyalkyl(C₁-C₃)alkyl[OCO(C₅-C₁₆), preferably (C₈-C₁₄), most preferably (C₁₀-C₁₂)alkyl]ₙ;
wherein
n is an integer 1 or 2; or
a derivative of ammonioalkyl(C₁-C₃)alkylPy;
wherein
Py means a pyridinium or a (C₁-C₃)alkylN⁺tri(C₁-C₁₀), preferably (C₃-C₆)alkyl;
R₄ is H, (C₁-C₁₆), preferably (C₈-C₁₄), most preferably (C₁₀-C₁₂)alkyl or an alkyl group with a carbonyl function, selected from a group consisting of COO(C₁-C₁₀), preferably (C₃-C₆)alkyl, COOsteryl, aryl and C₆H₄R₈;
wherein
R₈ is H, Cl, Br, I, CH₃, OCH₃, N(CH₃)₂, NO₂, OCHF₂;
or a heteryl group preferably pyridinium C₅H₄N⁺R₉;
wherein
R₉ is (C₁-C₁₆), preferably (C₈-C₁₄), most preferably (C₁₀-C₁₂)alkyl, aryl, aralkyl, alkoxycarbonylalkyl, cycloalkylcarbonylalkyl, (C₁-C₁₂), preferably (C₃-C₉), most preferably (C₅-C₇)alkylCOR₁₀ with a carbonylalkyl function;
wherein
R₁₀ is NH₂, O(C₁-C₁₆), preferably (C₈-C₁₄), most preferably (C₁₀-C₁₂)alkyl, aryl, O-steryl, OH, O—, or COR₁₁; wherein R₁₁ is OH, O—, O(C₁-C₁₆), preferably (C₈-C₁₄), most preferably (C₁₀-C₁₂)alkyl, O-aryl or N(R₁₂)₂;
wherein
R₁₂ is H, (C₁-C₁₆), preferably (C₈-C₁₄), most preferably (C₁₀-C₁₂)alkyl, pyridiniumalkyl, ammoniumalkyl, carbalkoxyalkyl or carboxyalkyl;
R₅ is ammonio, pyridinio selected from a group consisting of C₅H₅N⁺—, [(C₁-C₁₀), preferably (C₃-C₉), most preferably (C₅-C₇)alkyl]₃N⁺, (C₁-C₁₆), preferably (C₈-C₁₄), most preferably (C₁₀-C₁₂)alkoxycarbonylmethylpyridyl or C(═Y)—(Z)ₙR₇ with a
carbonyl function;
wherein
Y is O or S;
Z is O, S or NH;
n is an integer o or 1;
R₇ is as defined above; and
R₆ is (C₁-C₁₀), preferably (C₃-C₆)alkyl, CH₂X;
wherein
X is pyridinio selected from a group consisting of pyridinio (C₅H₅N⁺), substituted pyridinio, trialkyl(C₁-C₁₀), preferably (C₃-C₆)ammonio and aryl;
R₂ is conveniently the same as R₆ and R₃ the same as R₅
Optionally R₅ and R₆ may taken together form a dioxosulfaindeno group SO₂C₆H₄; and/or R₁ and R₂ taken together form a carbonylmethylthio group.
In the 1,4-dihydropyridine derivatives of the present invention each ammonium and/or pyridinium group is provided with a counter- ion W—, wherein W means a halide, selected from a group consisting of I, Br and Cl; perchlorate (ClO₄), sulfate (1/2SO₄), phosphate (1/3PO₄ or H₂PO₄).

2. The composition according to claim 1, characterized in that
R₂ is either the same as or different from R₆ and is methyl, pyridiniomethylbromide, trialkylammoniomethylbromide, carbamoylmethylthio or alkylcarbamoylmethylthio;
R₃ is either the same as or different from R₅ and is octyloxypropyloxycarbonyl, nonyloxypropyloxycarbonyl, decyloxypropyloxycarbonyl, undecyloxypropyloxycarbonyl, dodecyloxypropyloxycarbonyl, tridecyloxypropyloxycarbonyl, tetradecyloxypropyloxycarbonyl, pentadecyloxypropyloxycarbonyl, hexadecyloxypropyloxycarbonyl, octyloxypropyloxycarbonyl, nonyloxypropyloxycarbonyl, decyloxypropyloxycarbonyl, undecyloxypropyloxycarbonyl, dodecyloxypropyloxycarbonyl, tridecyloxypropyloxycarbonyl, tetradecyloxypropyloxycarbonyl, pentadecyloxypropyloxycarbonyl, hexadecyloxypropyloxycarbonyl, pentadecyloxycarbonylpropyloxycarbonyl, octadecyloxycarbonyl, nonyloxycarbonyl, decyloxycarbonyl, undecyloxycarbonyl, dodecyloxycarbonyl, tridecyloxycarbonyl, tetradecyloxycarbonyl, pentadecyloxycarbonyl, hexadecyloxycarbonyl, propyloxyethyloxycarbonyl, (2,3-dipentadecyoxycarbonyl)propyloxycarbonyl, menthyloxycarbonyl, bornyloxycarbonyl, cholesteryloxycarbonyl, ethyloxycarbonyl, propyloxycarbonyl, cyclohexyl[2-isopropyl]carbonyl, decyloxycarbonyl, ethylthiocarbonyl, dodecylthiocarbonyl, carbamoyl, hexadecylamidocarbonyl, diethylamidocarbonyl, morpholidocarbonyl, pyridyl, pyridinium, pyridinio, triethylammonio, trioctylammonio, dimethyloctylammonio, triethylammonioethoxycarbonyl, dimethyloctylammonioethoxycarbonyl, pyridinioethoxycarbonyl, benzylamidocarbonyl;
R₄ is iodomethylpyridinium, bromononylpyridinium, bromohexadecylpyridinium, iodopropylpyridinium, iodocarbamoylmethylpyridinium, bromobutylpyridinium, phenyl, iodoacetonylpyridinium, bromonaphthacylpyridinium, bromoethoxycarbonylmethylpyridinium, bromophenacylpyridinium, ethoxycarbonylethylcarbamoyl, pyridinioethylamidocarbonyl or diethylcarbamoyl; and
R₁ is hydrogen, methyl, ethyl, butyl, dodecyl or benzyl.

3. The composition according to claim 1, characterized in, that the 1,4-dihydropyridine derivatives are selected from a group consisting of 1-methyl-3-(2′,6′-dimethyl-3′,5′-dipentadecyloxycarbonylethoxycarbonyl-1′,4′-dihydropyridyl-4′)-pyridinium iodide; 1-methyl-3-(2′, 6′-dimethyl-3′,5-dihexadecyloxypropyloxycarbonyl-1′,4′-dihydropyridyl-4′) -pyridinium iodide; 1-methyl-3- (2′,6′-dimethyl-3′,5′-dipentadecyloxycarbonylpropyloxycarbonyl-1′,4′-dihydropyridyl-4′)-pyridinium iodide; 1-methyl-3-(2′,6′-dimethyl-3′,5′-dinonyloxycarbonyl-1′,4′-dihydropyridyl-4′)-pyridinium iodide; 1-methyl-3- (2′,6′-dimethyl-3′,5′-didodecyloxycarbonyl-1′,4′-dihydropyridyl-4′)-pyridinium iodide; 1-methyl-3-(2′,6′-dimethyl-3′,5′-ditetradecyloxycarbonyl-1′,4′-dihydropyridyl-4′)-pyridinium iodide; 1-methyl-3-(2′,6′-dimethyl-3′,5′-dihexadecyloxycarbonyl-1′,4′-dihydropyridyl-4′)-pyridinium iodide; 1-methyl-3-(2′,6′-dimethyl-3′,5′-dipropoxyethoxycarbonyl-1′,4′-dihydropyridyl-4′)-pyridinium iodide; 1-methyl-3-(2′,6′-dimethyl-3′,5′-di(2,3-dipentadecyloxycarbonyl)propyloxycarbonyl-1′,4′-dihydropyridyl-4′)pyridinium iodide; 1-methyl-3-(2′,6′-dimethyl-3′,5′-dimenthyloxycarbonyl-1′,4′-dihydropyridyl-4′)-pyridinium iodide; 1-methyl-3-(2′,6′-dimethyl-3′,5′-dibornyloxycarbonyl-1′,4′-dihydropyridyl-41)-pyridinium iodide; 1-methyl-3-(2′,6′-dimethyl-3′,5′-dicholesteryl-oxycarbonyl-1′,4′-dihydropyridyl-4′)-pyridinium iodide; 1-nonyl-3-(2′,6′-dimethyl-3′,5′-diethoxycarbonyl-1′,4′-dihydropyridyl-1′,4′)-pyridinium bromide; 1-nonyl-3-(2′,6′-dimethyl-3′,5′-ditetradecyloxycarbonyl-1′,4′-dihydropyridyl-4′)-pyridinium bromide; 1-hexadecyl-4-(2′,6′-dimethyl-3′,5′-dietoxycarbonyl-1′,4′-dihydropyridyl-4′)-pyridinium bromide; 1-hexadecyl-3-(2′,6′-dimethyl-3′,5′-dipropoxyethoxycarbonyl-1′,4′-dihydropyridyl-4′)-pyridinium bromide; 1-hexadecyl-3-(2′,6′-dimethyl-3′,5′-ditetradecyloxycarbonyl-1′,4′-dihydropyridyl-4′)-pyridinium bromide; 1-hexadecyl-3-(2′,6′-dimethyl-3′,5′-dimenthyloxycarbonyl-1′,4′-dihydropyridyl-4′)-pyridinium bromide; 1-propyl-3-(2′,6′-dimethyl-3′,5′-dipropoxyethoxycarbonyl-1′,4′-dihydropyridyl-4′)-pyridinium iodide; 1-carbamoylmethyl-3-(2′,6′-dimethyl-3′,5′-dihexadecyloxycarbonyl-1′,4′-dihydropyridyl-4′)-pyridinium iodide; 1-butyl-3-(2′,6′-dimethyl-3′,5′-ditetradecyloxycarbonyl-1′,4′- dihydropyridyl-4′)-pyridinium bromide; 1,1′-[(3,5-didecyloxycarbonyl-4-phenyl-1,4-dihydropyridine-2,6-diyl)dimethylene]bispyridinium dibromide; 1,1′-[(3,5-didodecyloxycarbonyl-4-phenyl-1,4-dihydropyridine-2,6-diyl)dimethylene]bispyridinium dibromide; 1,1′-[(3,5-ditetradecyloxycarbonyl-4-phenyl-1,4-dihydropyridine-2,6-diyl)dimethylene]bispyridinium dibromide; 1,1′-[(3,5-dihexadecyloxycarbonyl-4-phenyl-1,4-dihydropyridine-2,6-diyl)dimethylene]bispyridinium dibromide; 1-hexadecyl-3-[2′,6′-dimethyl-3′,5′-di(ethylthiocarbonyl)-1′,4′-dihydropyridyl-4′]-pyridinium bromide; N,N′-[(3,5-didecyloxycarbonyl-4-phenyl-1,4-dihydropyridine-2,6-diyldimethylene]bis-N,N-dimethyloctylammonium dibromide; N,N′-[(4-(2-difluoromethoxyphenyl)-3,5-dimethoxycarbonyl-1,4-dihydropyridine-2,6-diyl)dimethylene]bistriethylammonium) dibromide; 1,1′-[(4-difluoromethoxyphenyl-3,5-dimethoxycarbonyl-1-methyl-1,4-dihydropyridine-2,6-diyl)dimethylene]bis dibromide; 2-carbamoylmethylthio-3-cyano-5-[(N-alkoxycarbonyl)-4-pyridyl]-6-methyl-4-(3-nitrophenyl)-1,4-dihydropyridine bromide; 6-[(N-alkoxycarbonylmethyl)-4-pyridyl]-5-methyl-7-(3-nitrophenyl)-3-oxo-2,3-dihydro-7H-thiazolo[3,2-a]pyridine-8-carbonitrite bromide; 1-hexadecyl-3-{3-(1-adamanthyloxycarbonyl)-1,4-dihydrobenzothieno[3,2-b]-pyridyl-5,5 dioxide-4}-pyridinium bromide; (N,N′-[(-2,6-dimethyl-4-o-methoxyohenyl-1,4-dihydropyridine-3,5-diyl)ethoxycarbonyl]bis-N,N-dimethyloctylammonium diiodide; 3,5-dioctadec-9′-enyloxycarbonyl-4-phenyl-1,4-dihydropyridin-2,6-bis(1,1′-methylenpyridinium)dihydrobromide; 1,1′-[(3,5-didodecyloxycarbonyl-4-phenyl-1-methyl-1,4-dihydropyridine-2,6-diyl)dimethylene]bispyridinium dibromide; 1,1′-[(3,5-didodecyloxycarbonyl-4-phenyl-1-hexyl-1,4-dihydropyridine-2,6-diyl)dimethylene]bispyridinium dibromide; 1,1′-[(3,5-dihexadecylaminocarbonyl-4-phenyl-1-hexyl-1,4-dihydropyridine-2,6-diyl)dimethylene]bispyridinium dibromide; 1,1′-[(3,5-di-N,N-dimethyloctylammonioethoxycarbonyl-4-phenyl-1,4-dihydropyridine-2,6-diyl)dimethylene]bispyridinium tetrabromide; 1,1′-[(2,6-dimethyl-4-phenyl-1-methyl-1,4-dihydropyridine-3,5-diyl)ethoxycarbonyl]bispyridinium diiodide; 1,1′-[(315-didodecyloxycarbonyl-4-ethoxycarbonyl-1,4-dihydropyridine-2,6-diyl)dimethylenelbispyridinium dibromide; 1,1!-[(4-alkoxy-3,5-didodecyloxycarbonyl-4-phenyl-1,4-dihydropyridine-2,6-diyl)dimethylene]bispyridinium dibromide; 1,1′-[(4-alkylamidocarbonyl-3,5-didodecyloxycarbonyl-1,4-dihydropyridine-2,6-diyl)dimethylene]bispyridinium dibromide; 1-alkoxycarbonylmethyl-3-(3,5-didodecyloxycarbonyl-2,6-dipyridiniomethyl-1,4-dihydropyridine-2,6-diyl)dimethylene]bispyridinium dibromide; 4-alkylamidocarbonyl-3,5-didodecyloxycarbonyl-2,6-dipyridiniomethyl-1,4-dihydropyridyl-4)pyridinium) tribromide; 1-ethylamidocarbonyl-3-(3′,5′-didodecyloxycarbonyl-2,6-dihydropyridiniomethyl-1,4-dihydropyridyl-4)pyridinium tribromide; 4-phenyl-3,5-diethyloxycarbonyl-1-phenyl-1,4-dihydropyridine2,6-bis-(1,1′-methylenpyridiinium)dibromide; and
4-phenyl-3,5-didodecyloxycarbonyl-1-phenyl-1,4-dihydropyridine-2,6-bis-(1,1′-methylenpyridinium) dibromide.

4. The composition according to claims 1-3, characterized in, that the nucleotide containing compound is a nucleic acid, DNA, RNA, oligonucleotides, plasmids, vectors, chimeric DNA/RNA constructs, ribozymes as well as fragments and/or modifications thereof.

5. The composition according to claim 1, characterized in, that the composition comprises optional compatible additives selected from a group consisting of cationic liposomes lacking the 1,4-dihydropyridine structure, fusogenic peptides, targeting agents, antibodies, membrane active proteins, surfactants and compounds, which prolong the half-life in blood or serum, buffered or non-buffered aqueous solutions.

6. The composition according to claim 5, characterized in that the cationic liposomes lacking the 1,4-dihydropyridine structure is dioleylphosphatidylethanolamine (DOPE).

7. The composition according to claim 5, characterized in that the compounds which prolong the half-life in blood circulation is polyethylenglycol (PEG) or fragments thereof.

8. The composition according to claims 1-5, characterized in that the composition further comprises pharmaceutically acceptable compatible additives or means for administration.

9. The composition according to claim 8, characterized in that the means for administration comprises a solid controlled release matrix or device.

10. An 1,4-dihydropyridine derivative with the general formula I, characterized in, that 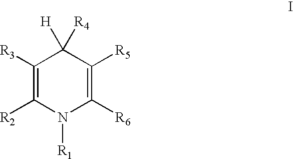
wherein
R₁ is hydrogen, (C₁-C₁₆), preferably (C₈-C₁₄), most preferably (C₁₀-C₁₂)alkyl, aralkyl or aryl, selected from a group consisting of phenyl, substituted phenyl, naphthyl, acylCO(C₁-C₁₆), preferably (C₈-C₁₄), most preferably (C₁₀-C₁₂)alkyl and COaryl;
R₂ is (C₁-C₁₀), preferably (C₁-C₃)alkyl or CH₂X;
wherein
X is pyridinio (C₅H₅N⁺), substituted pyridinio, diazinio (C₄H₄N₂⁺), substituted diazinio, trialkyl (C₁-C₁₀), preferably (C₁-C₃)ammonio, a (C₁-C₁₆), preferably (C₈-C₁₄), most preferably (C₁₀-C₁₂)alkylthio group or an alkylthio group with a carbonyl function, selected from a group consisting of S(CH₂)ₙCONH₂, S(CH₂)ₙCOAr and S(CH₂)ₙCOO(C₁-C₁₆), preferably (C₈-C₁₄), most preferably (C₁₀-C₁₂)alkyl;
wherein
n is an integer from 1 to 16, preferably 8-14, most preferably 10-12;
R₃ is a cyano or nitril group or C(═Y)—(Z)ₙR₇ with a carbonyl function;
wherein
Y is O or S;
Z is O, S or NH or NR₇;
n is an integer 0 or 1; and
R₇ is saturated or unsaturated (C₁-C₁₈), preferably (C₈-C₁₄), most preferably (C₁₀-C₁₂)alkyl, a derivative of cyclohexane, a terpene selected from a group consisting of bornyl, i-bornyl, menthyl, steryl, cholesteryl, adamantyl, aralkyl(C₁-C₃)alkylAr;
wherein
Ar means aryl, alkoxyalkyl(C₁-C₆), preferably (C₁-C₃)alkyl-O—(C₁-C₃)alkyl, alkanoyloxyalkyl(C₁-C₃)alkyl[OCO(C₅-C₁₆), preferably (C₈-C₁₄), most preferably (C₁₀-C₁₂)alkyl]ₙ;
wherein
n is an integer 1 or 2; or
a derivative of ammonioalkyl(C₁-C₃)alkylPy;
wherein
Py means a pyridinium or a (C₁-C₃)alkylN⁺tri(C₁-C₁₀), preferably (C₃-C₆)alkyl;
R₄ is H, (C₁-C₁₆), preferably (C₈-C₁₄), most preferably (C₁₀-C₁₂)alkyl or an alkyl group with a carbonyl function, selected from a group consisting of COO(C₁-C₁₀), preferably (C₃-C₆)alkyl, COOsteryl, aryl and C₆H₄R₈;
wherein
R₈ is H, Cl, Br, I, CH₃, OCH₃, N(CH₃)₂, NO₂, OCHF₂; or a heteryl group preferably pyridinium C₅H₄N⁺R₉;
wherein
R₉ is (C₁-C₁₆), preferably (C₈-C₁₄), most preferably (C₁₀-C₁₂)alkyl, aryl, aralkyl, alkoxycarbonylalkyl, cycloalkylcarbonylalkyl, (C₁-C₁₂), preferably (C₃-C₉), most preferably (C₅-C₇)alkylCOR₁₀ with a carbonylalkyl function;
wherein
R₁₀ is NH₂, O(C₁-C₁₆), preferably (C₈-C₁₄), most preferably (C₁₀-C₁₂)alkyl, aryl, O-steryl, OH, O—, or COR₁₁; wherein
R₁₁ is OH, O—, O(C₁-C₁₆), preferably (C₈-C₁₄), most preferably (C₁₀-C₁₂)alkyl, O-aryl or N(R₁₂)₂;
wherein
R₁₂ is H, (C₁-C₁₆), preferably (C₈-C₁₄), most preferably (C₁₀-C₁₂)alkyl, pyridiniumalkyl, ammoniumalkyl, carbalkoxyalkyl or carboxyalkyl;
R₅ is ammonio, pyridinio selected from a group consisting of C₅H₅N^{+—, [(C}₁-C₁₀), preferably (C₃-C₉), most preferably (C₅-C₇)alkyl]₃N⁺, (C₁-C₁₆), preferably (C₈-C₁₄), most preferably (C₁₀-C₁₂)alkoxycarbonylmethylpyridyl or C(═Y)—(Z)ₙR₇ with a
carbonyl function;
wherein
Y is O or S;
Z is O, S or NH;
n is an integer 0 or 1;
R₇ is as defined above; and
R₆ is (C₁-C₁₀), preferably (C₃-C₆)alkyl, CH₂X;
wherein
X is pyridinio selected from a group consisting of pyridinio (C₅H₅N⁺), substituted pyridinio, trialkyl(C₁-C₁₀), preferably (C₃-C₆)ammonio and aryl;
R₂ is conveniently the same as R₆ and R₃ the same as R₅
Optionally RS and R₆ may taken together form a dioxosulfaindeno group SO₂C₆H₄; and/or R, and R₂ taken together form a carbonylmethylthio group.
In the 1,4-dihydropyridine derivatives of the present invention each ammonium and/or pyridinium group is provided with a counter- ion W—, wherein W means a halide, selected from a group consisting of I, Br and Cl; perchlorate (ClO₄), sulfate (1/2SO₄), phosphate (1/3PO₄ or H₂PO₄).

11. The 1,4-dihydropyridine according to claim 10, characterized in, that
R₂ is either the same as or different from R₆ and is methyl, pyridiniomethylbromide, trialkylammoniomethylbromide, carbamoylmethylthio or alkylcarbamoylmethylthio;
R₃ is either the same as or different from R₅ and is octyloxypropyloxycarbonyl, nonyloxypropyloxycarbonyl, decyloxypropyloxycarbonyl, undecyloxypropyloxycarbonyl, dodecyloxypropyloxycarbonyl, tridecyloxypropyloxycarbonyl, tetradecyloxypropyloxycarbonyl, pentadecyloxypropyloxycarbonyl, hexadecyloxypropyloxycarbonyl, octyloxypropyloxycarbonyl, nonyloxypropyloxycarbonyl, decyloxypropyloxycarbonyl, undecyloxypropyloxycarbonyl, dodecyloxypropyloxycarbonyl, tridecyloxypropyloxycarbonyl, tetradecyloxypropyloxycarbonyl, pentadecyloxypropyloxycarbonyl, hexadecyloxypropyloxycarbonyl, pentadecyloxycarbonylpropyloxycarbonyl, octadecyloxycarbonyl, nonyloxycarbonyl, decyloxycarbonyl, undecyloxycarbonyl, dodecyloxycarbonyl, tridecyloxycarbonyl, tetradecyloxycarbonyl, pentadecyloxycarbonyl, hexadecyloxycarbonyl, propyloxyethyloxycarbonyl, (2,3-dipentadecyoxycarbonyl)propyloxycarbonyl, menthyloxycarbonyl, bornyloxycarbonyl, cholesteryloxycarbonyl, ethyloxycarbonyl, propyloxycarbonyl, cyclohexyl[2-isopropyl]carbonyl, decyloxycarbonyl, ethylthiocarbonyl, dodecylthiocarbonyl, carbamoyl, hexadecylamidocarbonyl, diethylamidocarbonyl, morpholidocarbonyl, pyridyl, pyridinium, pyridinio, triethylammonio, trioctylammonio, dimethyloctylammonio, triethylammonioethoxycarbonyl, dimethyloctylammonioethoxycarbonyl, pyridinioethoxycarbonyl, benzylamidocarbonyl;
R₄ is iodomethylpyridinium, bromononylpyridinium, bromohexadecylpyridinium, iodopropylpyridinium, iodocarbamoylmethylpyridinium, bromobutylpyridinium, phenyl, iodoacetonylpyridinium, bromonaphthacylpyridinium, bromoethoxycarbonylmethylpyridinium, bromophenacylpyridinium, ethoxycarbonylethylcarbamoyl, pyridinioethylamidocarbonyl or diethylcarbamoyl; and
R₁ is hydrogen, methyl, ethyl, butyl, dodecyl or benzyl.

12. The 1,4-dihydropyridine according to claim 10, characterized in, that they are selected from a group consisting of 1-methyl-3-(2′,6′-dimethyl-3′,5′-dipentadecyloxycarbonylethoxycarbonyl-1′,4′-dihydropyridyl-4′)-pyridinium iodide; 1-methyl-3-(2′,6′-dimethyl-3′,5′-dihexadecyloxypropyloxycarbonyl-1′,4′-dihydropyridyl-4′)-pyridinium iodide; 1-methyl-3-(2′,6′-dimethyl-3′,5′-dipentadecyloxycarbonyl-propyloxycarbonyl-1′,4′,-dihydropyridyl-4′)-pyridinium iodide; 1-methyl-3-(2′,6′-dimethyl-3′,5′-dinonyloxycarbonyl-1′,4′-dihydropyridyl-4′)-pyridinium iodide; 1-methyl-3-(2′,6′-dimethyl-3′,5′-didodecyloxycarbonyl-1′,4′-dihydropyridyl-4′)-pyridinium iodide; 1-methyl-3-(2′,6′-dimethyl-3′,5′-ditetradecyloxycarbonyl-1′,4′- dihydropyridyl-4′)-pyridinium iodide; 1-methyl-3-(2′,6′-dimethyl-3,5′-dihexadecyloxycarbonyl-1′,4′- dihydropyridyl-4′)-pyridinium iodide; 1-methyl-3-(2′,6′-dimethyl-3′,5′-dipropoxyethoxycarbonyl-1′,4′-dihydropyridyl-4′)-pyridinium iodide; 1-methyl-3-(2′,6′-dimethyl-3′,5′-di(2,3-dipentadecyloxycarbonyl)propyloxycarbonyl-1′,4′-dihydropyridyl-4′)pyridinium iodide; 1-methyl-3-(2′,6′-dimethyl-3′,5′-dimenthyloxycarbonyl-1′,4′-dihydropyridyl-4′)-pyridinium iodide; 1-methyl-3-(2′,6′-dimethyl-3′,5′-dibornyloxycarbonyl-1′,4′-dihydropyridyl-4′)-pyridinium iodide; 1-methyl-3-(2′,6′-dimethyl-3,5′-dicholesteryl-oxycarbonyl-1′,4′-dihydropyridyl-4′)-pyridinium iodide; 1-nonyl-3-(2′,6′-dimethyl-3′,5′-diethoxycarbonyl-1′,4′-dihydropyridyl-4′)-pyridinium bromide; 2-nonyl-3-(2′,6′-dimethyl-3′,5′-ditetradecyloxycarbonyl-1′,4′-dihydropyridyl-4′)-pyridinium bromide; 1-hexadecyl-4-(2′,6′-dimethyl-3′,5′-dietoxycarbonyl-1′,4′-dihydropyridyl-41)-pyridinium bromide; 1-hexadecyl-3-(2′,6′-dimethyl-3′,5′-dipropoxyethoxycarbonyl-1′,4′-dihydropyridyl-4′)-pyridinium bromide; 1-hexadecyl-3-(2′,6′-dimethyl-3′,5′-ditetradecyloxycarbonyl-1′,4′-dihydropyridyl-4′)-pyridinium bromide; 1-hexadecyl-3-(2′,6′-dimethyl-3′,5′-dimenthyloxycarbonyl-1′,4′-dihydropyridyl-4′)-pyridinium bromide; 1-propyl-3-(2′,6′-dimethyl-3′,5′-dipropoxyethoxycarbonyl-1′,4′-dihydropyridyl-4′)-pyridinium iodide; 1-carbamoylmethyl-3-(2′,6′-dimethyl-3′,5′-dihexadecyloxycarbonyl-1′,4′-dihydropyridyl-4′)-pyridinium iodide; 1-butyl-3-(2′, g-dimethyl-3′,5′-ditetradecyloxycarbonyl-1′,4′- dihydropyridyl-4′)-pyridinium bromide; 1,1′-[(3,5-didecyloxycarbonyl-4-phenyl-1,4-dihydropyridine-2,6-diyl)dimethylene]bispyridinium dibromide; 1,1′-[(3,5-didodecyloxycarbonyl-4-phenyl-1,4-dihydropyridine-2,6-diyl)dimethylene]bispyridinium dibromide; 1,1′-[(3,5-ditetradecyloxycarbonyl-4-phenyl-1,4-dihydropyridine-2,6-diyl)dimethylene]bispyridinium dibromide; 1,1′-[(3,5-dihexadecyloxycarbonyl-4-phenyl-1,4-dihydropyridine-2,6-diyl)dimethylene]bispyridinium dibromide; 1-hexadecyl-3-[2′,6′-dimethyl-3′,5′-di(ethylthiocarbonyl)-1′,4′-dihydropyridyl-41]-pyridinium bromide; N,N′-[(3,5-didecyloxycarbonyl-4-phenyl-1,4-dihydropyridine-2,6-diyldimethylene]bis-N,N-dimethyloctylammonium dibromide; N,N′-[(4-(2-difluoromethoxyphenyl)-3,5-dimethoxycarbonyl-1,4-dihydropyridine-2,6-diyl)dimethylene]bistriethylammonium) dibromide; 1,1′-[(4-difluoromethoxyphenyl-3,5-dimethoxycarbonyl-1-methyl-1,4-dihydropyridine-2,6-diyl)dimethylene]bis-dibromide; 2-carbamoylmethylthio-3-cyano-5-[(N-alkoxycarbonyl)-4-pyridyl]-6-methyl-4-(3-nitrophenyl)-1,4-dihydropyridine bromide; 6-[(N-alkoxycarbonylmethyl)-4-pyridyl]-5-methyl-7-(3-nitrophenyl)-3-oxo-2,3-dihydro-7H-thiazolo[3,2-a]pyridine-8-carbonitrile bromide; 1-hexadecyl-3-{3-(1′-adamanthyloxycarbonyl)-1,4-dihydrobenzothieno[3,2-b]-pyridyl-5,5 dioxide-4}-pyridinium bromide; (N,N′-[(-2,6-dimethyl-4-o-methoxyohenyl-1,4-dihydropyridine-3,5-diyl)ethoxycarbonyl]bis-N,N-dimethyloctylammonium diiodide; 3,5-dioctadec-9′-enyloxycarbonyl-4-phenyl-1,4-dihydropyridin-2,6-bis(1,1′-methylenpyridinium)dihydrobromide; 1,1′-[(3,5-didodecyloxycarbonyl-4-phenyl-1-methyl-1,4-dihydropyridine-2,6-diyl)dimethylene]bispyridinium dibromide; 1,1′-[(3,5-didodecyloxycarbonyl-4-phenyl-1-hexyl-1,4-dihydropyridine-2,6-diyl)dimethylene]bispyridinium dibromide; 1,1′-[(3,5-dihexadecylaminocarbonyl-4-phenyl-1-hexyl-1,4-dihydropyridine-2,6-diyl)dimethylene]bispyridinium dibromide; 1,1′-[(3,5-di-N,N-dimethyloctylammonioethoxycarbonyl-4-phenyl-1,4-dihydropyridine-2,6-diyl)dimethylene]bispyridinium tetrabromide; 1,1′-[(2,6-dimethyl-4-phenyl-1-methyl-1,4-dihydropyridine-3,5-diyl)ethoxycarbonyl]bispyridinium diiodide; 1,1′-[(3,5-didodecyloxycarbonyl-4-ethoxycarbonyl-1,4-dihydropyridine-2,6-diyl)dimethylene]bispyridinium dibromide; 1,1′-[(4-alkoxy-3,5-didodecyloxycarbonyl-4-phenyl-1,4-dihydropyridine-2,6-diyl)dimethylenelbispyridinium dibromide; 1,11-[(4-alkylamidocarbonyl-3,5-didodecyloxycarbonyl-1,4-dihydropyridine-2,6-diyl)dimethylene]bispyridinium dibromide; 1-alkoxycarbonylmethyl-3-(3,5-didodecyloxycarbonyl-2,6-dipyridiniomethyl-1,4-dihydropyridine-2,6-diyl)dimethylene]bispyridinium dibromide; 4-alkylamidocarbonyl-3,5-didodecyloxycarbonyl-2,6-dipyridiniomethyl-1,4-dihydropyridyl-4)pyridinium) tribromide; i-ethylamidocarbonyl-3-(3,5-didodecyloxycarbonyl-2,6-dihydropyridiniomethyl-1,4-dihydropyridyl-4)pyridinium tribromide; 4-phenyl-3,5-diethyloxycarbonyl-1-phenyl-1,4-dihydropyridine2,6-bis-(1,1′-methylenpyridiinium) dibromide; and 4-phenyl-3,5-didodecyloxycarbonyl-1-phenyl-1,4-dihydropyridine-2,6-bis-(1,1′-methylenpyridinium) dibromide.

13. The 1,4-dihydropyridine according to claim 10, characterized in, that it is 1-methyl-3-(2′,6′-dimethyl-3′,5′-dipentadecyloxycarbonylethoxycarbonyl-1′,4′-dihydropyridyl-4′)-pyridinium iodide.

14. The 1,4-dihydropyridine according to claim 10, characterized in, that it is 1-methyl-3-(2′,6′-dimethyl-3′,5′-didodecyloxycarbonyl-1′,4′-dihydropyridyl-4′)-pyridinium iodide.

15. The 1,4-dihydropyridine according to claim 10, characterized in, that it is 1-methyl-3-(2′, 6′-dimethyl-3′,5′-ditetradecyloxycarbonyl-1′,4′-dihydropyridyl-4′)-pyridinium iodide.

16. The 1,4-dihydropyridine according to claim 10, characterized in, that it is 1-carbamoylmethyl-3-(2′,6′-dimethyl-3′,5′-dihexadecyl-oxycarbonyl-1′,4′-dihydropyridyl-4′)-pyridinium iodide.

17. The 1,4-dihydropyridine according to claim 10, characterized in, that it is 1,1′-[(3,5-didodecyloxycarbonyl-4-phenyl-1,4-dihydropyridine-2,6-diyl) dimethylene]bispyridinium dibromide.

18. The 1,4-dihydropyridine according to claim 10, characterized in, that it is 1,1′-[(3,5-ditetradecyloxycarbonyl-4-phenyl-1,4-dihydropyridine-2,6-diyl) dimethylene]bispyridinium dibromide.

19. The 1,4-dihydropyridine according to claim 10, characterized in, that it is N,N-(3,5-Didecyloxycarbonyl-4-phenyl-1,4-dihydro-pyridine-2,6-diyldimethylbis-dimethyloctylammonium dibromide.

20. A method for preparing liposomes for the manufacturing the composition according to claims 1-9, characterized in, that the 1,4-dihydropyridine derivatives according to claims 10-19 are depending upon their properties either dissolved directly or after having been dissolved in a non-polar solvent, removed by evaporation, in an aqueous solution and the mixture is vortexed or sonicated.

21. The method according to claim 20, characterized in, that the aqueous solution is a buffer or sugar solution.

22. A method for introducing nucleotide containing compounds into a target cell and its nucleus, characterized in, that the compositions according to any of claims 1-9 as such or in combination with pharmaceutically acceptable additives compatible with the route of administration are placed in contact with body fluids or tissues containing the target cells.

23. The method according to claim 22, characterized in, that the compositions according to any of claims 1-9 are locally administered in a controlled release matrix during a surgical intervention or into a body cavity.

24. Use of the amphiphilic 1,4-dihydropyridine derivatives according to any of the claims 10-19 for delivering nucleotide containing compounds into a target cell and/or its nucleus.

25. Use of the amphiphilic 1,4-dihydropyridine derivatives according to claims 10-19 for manufacturing gene delivery systems or vehicles for transporting nucleotide containing compounds into a target cell and its nucleus.
